(19) United States
(12) Patent Application Publication
(10) Pub. No.: US 2020/0281857 A1
(43) Pub. Date: Sep. 10, 2020

(54) THERAPEUTIC COMPOUND FORMULATIONS

(71) Applicant: Dauntless 1, Inc., San Diego, CA (US)

(72) Inventors: Olivier LAURENT, San Diego, CA (US); Joel F. MARTIN, Del Mar, CA (US)

(73) Assignee: Dauntless 1, Inc., San Diego, CA (US)

(21) Appl. No.: 16/766,213

(22) PCT Filed: Nov. 21, 2018
(86) PCT No.: PCT/US2018/062326

§ 371 Date: May 21, 2020

Related U.S. Application Data
(60) Provisional application No. 62/689,744, filed on Jun. 25, 2018.
(60) Provisional application No. 62/634,753, filed on Feb. 23, 2018.
(60) Provisional application No. 62/590,152, filed on Nov. 22, 2017.

(51) Int. Cl. A61K 9/16; A61K 38/31

(52) U.S. Cl. CPC A61K 9/1647; A61K 45/06; A61K 38/31; A61K 9/1617

(57) ABSTRACT

Provided herein are microspheres (e.g., single emulsion microspheres) comprising a therapeutic compound or pharmaceutically acceptable salt thereof, one or more polymers, and optionally a polyol, as well as methods of preparation, methods of use, and pharmaceutical compositions related thereto.

### CROSS REFERENCE TO RELATED APPLICATIONS

This application claims the priority benefit of U.S. Provisional Application Ser. No. 62/590,152, filed Nov. 22, 2017; 62/634,753, filed Feb. 23, 2018; and 62/689,744, filed Jun. 25, 2018, each of which is hereby incorporated by reference in its entirety.

### FIELD

The present disclosure relates to microspheres (e.g., single emulsion microspheres) comprising a therapeutic compound or pharmaceutically acceptable salt thereof, one or more polymers, and optionally a polyol, as well as methods of preparation, methods of use, and pharmaceutical compositions related thereto.

### BACKGROUND

Drug formulations such as microspheres are important for modulating the pharmacokinetic properties of the drug, such as release of the active compound. A variety of microspheres and methods of microsphere preparation have been described. See, e.g., U.S. Pat. Nos. 5,639,480 and 8,916,196. Double emulsion, water/oil/water microspheres have often been used for a variety of hydrophilic drug compounds. These are typically formed by emulsifying an aqueous solution of the hydrophilic drug with a solution of a polymer in organic solvent by high shear mixing (e.g., 20,000 rpm), generating an unstable water/oil emulsion. This unstable emulsion is then usually further emulsified in water, leading to a water/oil/water double emulsion that is then hardened by solvent exchange, and lyophilized into dry microspheres.

However, this high shear, high energy process is difficult to control or reproduce, leading to a heterogeneous preparation of double emulsion microspheres. One example of such a drug product is SANDOSTATIN® LAR depot (octreotide acetate). This drug is formulated in a double emulsion microsphere for a long release, thereby decreasing the frequency of administration. However, SANDOSTATIN® LAR is notoriously difficult to administer by injection; it frequently blocks flow through the needle (e.g., low syringeability), requiring thicker needles and leading to more painful injections for the patient. SANDOSTATIN® LAR is also characterized by low loading of the octreotide in the microspheres, necessitating a larger volume for injection that also causes significant pain during injection and can potentially lead to formation of painful nodules at the injection site. This further precludes subcutaneous injection in favor of more painful intramuscular injection. Finally, double emulsion microspheres are difficult to manufacture, requiring costly investment and poor process control.

As such, a demand exists for improved microsphere formulations characterized by improved pharmacokinetic properties, increased loading, a more uniform size distribution of particles, and easier, well-controlled manufacturing. Such properties would not only provide less-expensive, more-reliable manufacturing, but they would also allow for smaller injection needles (due, e.g., to higher drug loading and smaller, more uniform microspheres), thereby lessening the pain and inconvenience of injections. For example, if more compound can be loaded into each microsphere while keeping the pharmacokinetic properties of the drug (such as pharmacokinetic burst and subsequent release) at acceptable levels, then less material needs to be injected per administration.

All references cited herein, including patent applications, patent publications, non-patent literature, and UniProtKB/Swiss-Prot Accession numbers are herein incorporated by reference in their entirety, as if each individual reference were specifically and individually indicated to be incorporated by reference.

### SUMMARY

To meet these and other demands, provided herein is a microsphere comprising: a therapeutic compound or pharmaceutically acceptable salt thereof having a first pI; and a first polymer, wherein the polymer has a second pI at least 1.5 units lower than the first pI; wherein the microsphere is a single emulsion microsphere.

In some embodiments, the microsphere further comprises a second polymer, wherein first polymer has a lower molecular weight than the second polymer. In some embodiments, the pharmacokinetic burst in serum per mg of the therapeutic compound or salt for the microsphere is equal to or less than the pharmacokinetic burst in serum per mg of the therapeutic compound or salt for a reference microsphere. In some embodiments, the reference microsphere comprises the second polymer but lacks the first polymer. In some embodiments, the reference microsphere comprises the therapeutic compound or salt at a lower loading level than the microsphere. In some embodiments, the reference microsphere is a double emulsion microsphere. In some embodiments, the microsphere further comprises a polyol. In some embodiments, the pharmacokinetic burst in serum per mg of the therapeutic compound or salt for the microsphere is equal to or less than the pharmacokinetic burst in serum per mg of the therapeutic compound or salt for a reference microsphere, wherein the reference microsphere comprises the therapeutic compound and the polymer but lacks the polyol. In some embodiments, degradation of the therapeutic compound or salt in the microsphere is less than degradation of the therapeutic compound or salt in a reference microsphere, wherein the reference microsphere comprises the therapeutic compound and the polymer but lacks the polyol. In some embodiments, burst AUC in serum per mg of the therapeutic compound or salt for the microsphere is equal to or less than burst AUC in serum per mg of the therapeutic compound or salt for the reference microsphere. In some embodiments, burst Cmax in serum per mg of the therapeutic compound or salt for the microsphere is less than burst Cmax in serum per mg of the therapeutic compound or salt for the reference microsphere. In some embodiments, the first polymer has a molecular weight at least 10 kD lower than the second polymer. In some embodiments, the therapeutic compound or salt is greater than 5% by total weight of the microsphere. In some embodiments, the first polymer comprises at least one anionic terminus. In some embodiments, the first polymer comprises poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer. In some embodiments, the therapeutic compound or salt comprises at least one cationic moiety. In some embodiments, the microsphere is produced from a feed comprising the first polymer at a concentration of at least about 150 mg/mL and the therapeutic compound or salt at a concentration of at least about 10 mg/mL. In some embodiments, the microsphere is produced from a feed comprising the first polymer at a concentration of at least about 200 mg/mL and the therapeutic compound or salt at a concentration of at least about 20 mg/mL. In some embodiments, the microsphere is produced from a feed comprising the first polymer and the second polymer at a total concentration of at least about 150 mg/mL and the therapeutic compound or salt at a concentration of at least about 10 mg/mL. In some embodiments, the microsphere is produced from a feed comprising the first polymer and the second polymer at a total concentration of at least about 200 mg/mL and the therapeutic compound or salt at a concentration of at least about 20 mg/mL. In some embodiments, the molecular weight of the first polymer is less than or equal to 17 kD. In some embodiments, the second polymer comprises poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer. In some embodiments, the first and the second polymers both comprise PLGA. In some embodiments, the microsphere comprises the first polymer and the second polymer at a ratio of between about 20:80 and about 80:20 (first polymer:second polymer). In some embodiments, the microsphere comprises the first polymer and the second polymer at a ratio of about 65:35, about 50:50, or about 75:25 (first polymer:second polymer). In some embodiments, the polyol is glycerol. In some embodiments, the therapeutic compound comprises a therapeutic peptide. In some embodiments, the therapeutic peptide comprises at least two amino-containing amino acid side chains. In some embodiments, the therapeutic peptide has a length from 6 to 40 amino acids. In some embodiments, the therapeutic peptide has a length of 8 amino acids. In some embodiments, the therapeutic peptide is cyclic. In some embodiments, the therapeutic peptide is a somatostatin analog or a pharmaceutically acceptable salt thereof. In some embodiments, the therapeutic peptide is selected from the group consisting of somatostatin (SST-28), SST-14, lanreotide, octreotide, vapreotide, pasireotide, and pharmaceutically acceptable salts of any of the foregoing. In some embodiments, the therapeutic compound comprises a glucocorticoid, JAK inhibitor, or mTOR inhibitor. In some embodiments, wherein the therapeutic compound comprises a JAK inhibitor that inhibits JAK1, JAK3, JAK1 and JAK3, or JAK1, JAK2, and JAK3. In some embodiments, the therapeutic compound comprises a JAK inhibitor selected from the group consisting of ruxolitinib, tofacitinib, oclacitinib, baricitinib, filgotinib, gandotinib, lestaurtinib, momelotinib, pacritinib, PF-04965842, upadacitinib, peficitinib, fedratinib, cucurbitacin I, decernotinib, INCB018424, AC430, BMS-0911543, GSK2586184, VX-509, R348, AZD1480, CHZ868, PF-956980, AG490, WP-1034, JAK3 inhibitor IV, atiprimod, FM-381, SAR20347, AZD4205, ARN4079, NIBR-3049, PRN371, PF-06651600, JAK3i, JAK3 inhibitor 31, PF-06700841, NC1153, EP009, Gingerenone A, JANEX-1, cercosporamide, JAK3-IN-2, PF-956980, Tyk2-IN-30, Tyk2-IN-2, JAK3-IN1, WHI-P97, TG-101209, AZ960, NVP-BSK805, NSC 42834, FLLL32, SD 1029, WIH-P154, WHI-P154, TCS21311, JAK3-IN-1, JAK3-IN-6, JAK3-IN-7, XL019, MS-1020, AZD1418, WP1066, CEP33779, ZM 449829, SHR0302, JAK1-IN-31, WYE-151650, EXEL-8232, solcitinib, itacitinib, cerdulatinib, PF-06263276, delgotinib, AS2553627, JAK-IN-35, ASN-002, AT9283, diosgenin, JAK inhibitor 1, JAK-IN-1, LFM-A13, NS-018, RGB-286638, SB1317, curcumol, Go6976, JAK2 inhibitor G5-7, myricetin, and pyridine 6. In some embodiments, the microsphere is substantially free of small hydrocarbons (e.g., C₁-C₁₆ hydrocarbons, C₁-C₁₆ alkanes, heptane) and/or silicon oil.

Further provided herein are methods for preparing a plurality of microspheres (e.g., single emulsion microspheres), comprising the steps of: a) combining a first solvent and a therapeutic compound or pharmaceutically acceptable salt thereof to form a first mixture, wherein the compound or salt has a first pI; b) combining a second solvent and a polymer to form a second mixture, wherein the polymer has a second pI, and wherein the first pI is at least 1.5 units greater than the second pI; c) combining the first and second mixtures to form a feed; d) dispersing the combined first and second mixtures of step (c) (e.g., the feed) into an aqueous continuous phase to form a plurality of droplets; and e) hardening the plurality of droplets formed in step (d) to form the plurality of microspheres. Further provided herein is a plurality of microspheres, wherein the microspheres comprise: a therapeutic compound or pharmaceutically acceptable salt thereof having a first pI; and a polymer having a second pI, wherein the first pI is at least 1.5 units greater than the second pI. In some embodiments, at least 50%, at least 60%, at least 70%, at least 80%, at least 85%, or at least 90% of the microspheres of the plurality have a diameter within 10 μm, within 15 μm, or within 20 μm above or below a median diameter of the plurality. In some embodiments, the median diameter of the plurality is between about 5 μm to about 100 μm. In some embodiments, the microspheres further comprise a polyol (e.g., glycerol). In some embodiments, the microspheres of the plurality have at least 10% less, at least 15% less, at least 20% less, or at least 25% less pore surface area as measured by gas absorption isotherms using N₂, CO₂, or Hg as compared to a reference microsphere (e.g., a double emulsion microsphere, or a microsphere produced by a feed lacking the polyol such as glycerol but comprising the therapeutic compound or salt). In some embodiments, the polymer comprises poly(lactic-co-glycolic acid) (PLGA) with a carboxy-terminus, and the microsphere(s) comprise(s) the compound or salt at a concentration of at least 5% of the microsphere by total weight. In some embodiments, the microsphere(s) further comprise a polyol (e.g., glycerol).

Further provided herein are methods for preparing a plurality of microspheres (e.g., single emulsion microspheres), comprising the steps of: a) combining a first solvent and a therapeutic compound or pharmaceutically acceptable salt thereof to form a mixture, wherein the compound or salt has a first pI; b) combining a second solvent and a combination of polymers to form a second mixture, wherein a first polymer of the combination has a second pI, wherein the first pI is at least 1.5 units greater than the second pI, wherein the combination of polymers comprises two or more species of poly(lactic-co-glycolic acid) (PLGA), wherein at least one of the two or more species of PLGA comprises a carboxy-terminus, and wherein the two or more species of PLGA have a difference in minimum or maximum molecular weight of at least about 17 kD; c) combining the first and second mixtures to form a feed; d) dispersing the feed of step (c) into an aqueous continuous phase to form a plurality of droplets; and e) hardening the plurality of droplets formed in step (d) to form the plurality of microspheres. Further provided herein is a microsphere, comprising: a therapeutic compound or pharmaceutically acceptable salt thereof, wherein the compound or salt has a first pI; and a combination of polymers, wherein a first polymer of the combination has a second pI, wherein the first pI is at least 1.5 units greater than the second pI, wherein the combination of polymers comprises two or more species of poly(lactic-co-glycolic acid) (PLGA), wherein at least one of the two or more species of PLGA comprises a carboxy-terminus, and wherein the two or more species of PLGA have a difference in minimum or maximum molecular weight of at least about 7 kD, at least about 10 kD, at least about 17 kD, or at least about 20 kD. In some embodiments, the microsphere(s) further comprise a polyol (e.g., glycerol). In some embodiments, the species of PLGA having a smaller molecular weight comprises the carboxy-terminus.

Further provided herein are methods for increasing loading of a therapeutic compound into a microsphere (e.g., a single emulsion microsphere), the methods comprising: a) combining a first solvent and a therapeutic compound or pharmaceutically acceptable salt thereof to form a first mixture, wherein the compound or salt has a first pI; b) combining a second solvent and a polymer to form a second mixture, wherein the polymer has a second pI at least 1.5 units lower than the first pI; c) combining the first and second mixtures to form a feed; d) dispersing the combined first and second mixtures of step (c) into an aqueous continuous phase to form a droplet; and e) hardening the droplet formed in step (d) to form the single emulsion microsphere. In some embodiments, the microsphere comprises greater than 5% by total weight of the therapeutic compound or salt. Further provided herein are methods for reducing pharmacokinetic burst of a therapeutic compound-containing microsphere, the methods comprising: a) combining a first solvent and a therapeutic compound or pharmaceutically acceptable salt thereof to form a first mixture, wherein the compound or salt has a first pI; b) combining a second solvent, a first polymer, and a second polymer to form a second mixture, wherein the first polymer has a second pI at least 1.5 units lower than the first pI, and wherein the first polymer has a lower molecular weight than the second polymer; c) combining the first and second mixtures to form a feed; d) dispersing the combined first and second mixtures of step (c) into an aqueous continuous phase to form a droplet; and e) hardening the droplet formed in step (d) to form a single emulsion microsphere. In some embodiments, pharmacokinetic burst in serum per mg of the injected therapeutic compound or salt for the microsphere is equal to or less than pharmacokinetic burst in serum per mg of the injected therapeutic compound or salt for a reference microsphere, wherein the reference microsphere comprises the second polymer but lacks the first polymer and/or is a double emulsion microsphere, and wherein the reference microsphere comprises the therapeutic compound or salt at a lower loading level than the microsphere.

Further provided herein are methods for reducing pharmacokinetic burst of a therapeutic compound-containing microsphere, the methods comprising: a) combining a first solvent and a therapeutic compound or pharmaceutically acceptable salt thereof to form a first mixture, wherein the compound or salt has a first pI; b) combining a second solvent and a polymer to form a second mixture, wherein the polymer has a second pI at least 1.5 units lower than the first pI; c) combining the first and second mixtures to form a feed, wherein the feed comprises a polyol; d) dispersing the combined first and second mixtures of step (c) into an aqueous continuous phase to form a droplet; and e) hardening the droplet formed in step (d) to form the microsphere. In some embodiments, pharmacokinetic burst in serum per mg of the injected therapeutic compound or salt for the microsphere is less than pharmacokinetic burst in serum per mg of the injected therapeutic compound or salt for a reference microsphere, wherein the reference microsphere comprises the therapeutic compound and the polymer but lacks the polyol. Further provided herein are methods for improving stability of a therapeutic compound in a microsphere, the methods comprising: a) combining a first solvent and a therapeutic compound or pharmaceutically acceptable salt thereof to form a first mixture, wherein the compound or salt has a first pI; b) combining a second solvent and a polymer to form a second mixture, wherein the polymer has a second pI at least 1.5 units lower than the first pI; c) combining the first and second mixtures to form a feed, wherein the feed comprises a polyol; d) dispersing the combined first and second mixtures of step (c) into an aqueous continuous phase to form a droplet; and e) hardening the droplet formed in step (d) to form the microsphere. In some embodiments, degradation of the therapeutic compound or salt in the microsphere is less than degradation of the therapeutic compound or salt in a reference microsphere, wherein the reference microsphere comprises the therapeutic compound and the polymer but lacks the polyol.

Further provided herein are methods for preparing a single emulsion microsphere, comprising the steps of: a) combining a first solvent and a therapeutic compound or pharmaceutically acceptable salt thereof to form a first mixture, wherein the compound or salt has a first pI; b) combining a second solvent and a first polymer to form a second mixture, wherein the polymer has a second pI at least 1.5 units lower than the first pI; c) combining the first and second mixtures to form a feed; d) dispersing the combined first and second mixtures of step (c) (e.g., the feed) into an aqueous continuous phase to form a droplet; and e) hardening the droplet formed in step (d) to form the single emulsion microsphere.

In some embodiments of any of the above embodiments, step b) further comprises combining a second polymer with the second solvent and the first polymer to form the mixture. In some embodiments, the first polymer has a lower molecular weight than the second polymer. In some embodiments, the pharmacokinetic burst in serum per mg of the therapeutic compound or salt for the microsphere is equal to or less than pharmacokinetic burst in serum per mg of the therapeutic compound or salt for a reference microsphere. In some embodiments, the reference microsphere comprises the second polymer but lacks the first polymer. In some embodiments, the reference microsphere comprises the therapeutic compound or salt at a lower loading level than the microsphere formed in step (e). In some embodiments, the microsphere formed in step (e) does not induce a burst penalty as compared with the reference microsphere. In some embodiments, the reference microsphere is a double emulsion microsphere. In some embodiments, at least 80%, at least 85%, at least 90%, or at least 95% of the microspheres are 22-36 μm in diameter. In some embodiments, at least 50%, at least 55%, at least 60%, at least 65%, or at least 70% of the microspheres are 26-34 μm in diameter. In some embodiments, the feed further comprises a polyol. In some embodiments, the pharmacokinetic burst in serum per mg of the therapeutic compound or salt for the microsphere is less than the pharmacokinetic burst in serum per mg of the therapeutic compound or salt for a reference microsphere, wherein the reference microsphere comprises the therapeutic compound and the polymer but lacks the polyol. In some embodiments, degradation of the therapeutic compound or salt in the microsphere is less than degradation of the therapeutic compound or salt in a reference microsphere, wherein the reference microsphere comprises the therapeutic compound and the polymer but lacks the polyol. In some embodiments, burst AUC in serum per mg of the therapeutic compound or salt for the microsphere is equal to or less than burst AUC in serum per mg of the therapeutic compound or salt for the reference microsphere. In some embodiments, burst Cmax in serum per mg of the therapeutic compound or salt for the microsphere is less than burst Cmax in serum per mg of the therapeutic compound or salt for the reference microsphere. In some embodiments, the first polymer has a molecular weight at least 10 kD lower than the second polymer. In some embodiments, the microsphere comprises greater than 5% by total weight of the therapeutic compound or salt. In some embodiments, the first polymer comprises at least one anionic terminus. In some embodiments, the first polymer comprises poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer. In some embodiments, the therapeutic compound or salt comprises at least one cationic moiety. In some embodiments, the feed comprises the first polymer at a concentration of at least about 150 mg/mL and the therapeutic compound or salt at a concentration of at least about 10 mg/mL. In some embodiments, the feed comprises the first polymer at a concentration of at least about 200 mg/mL and the therapeutic compound or salt at a concentration of at least about 20 mg/mL. In some embodiments, the feed comprises the first polymer and the second polymer at a total concentration of at least about 150 mg/mL and the therapeutic compound or salt at a concentration of at least about 10 mg/mL. In some embodiments, the feed comprises the first polymer and the second polymer at a total concentration of at least about 200 mg/mL and the therapeutic compound or salt at a concentration of at least about 20 mg/mL. In some embodiments, the molecular weight of the first polymer is less than or equal to 17 kD. In some embodiments, the second polymer comprises poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer. In some embodiments, the first and the second polymers both comprise PLGA, and wherein the molecular weight of the first polymer is at least 10 kD lower than the molecular weight of the second polymer. In some embodiments, the microsphere comprises the first polymer and the second polymer at a ratio of between about 20:80 and about 80:20 (first polymer:second polymer). In some embodiments, the microsphere comprises the first polymer and the second polymer at a ratio of about 75:25 (first polymer:second polymer). In some embodiments, the microsphere comprises the first polymer and the second polymer at a ratio of about 65:35 (first polymer:second polymer). In some embodiments, the feed comprises the polyol at a concentration of about between about 0.3 mg/mL and about 1.2 mg/mL. In some embodiments, the feed comprises the polyol at a concentration of about 0.9 mg/mL. In some embodiments, the polyol is glycerol. In some embodiments, the therapeutic compound comprises a therapeutic peptide. In some embodiments, the therapeutic peptide comprises at least two amino-containing amino acid side chains. In some embodiments, the therapeutic peptide has a length from 6 to 40 amino acids. In some embodiments, the therapeutic peptide has a length of 8 amino acids. In some embodiments, the therapeutic peptide is cyclic. In some embodiments, the therapeutic peptide is a somatostatin analog or a pharmaceutically acceptable salt thereof. In some embodiments, the therapeutic peptide is selected from the group consisting of somatostatin (SST-28), SST-14, lanreotide, octreotide, vapreotide, pasireotide, and pharmaceutically acceptable salts of any of the foregoing. In some embodiments, the therapeutic compound comprises a glucocorticoid, JAK inhibitor, or mTOR inhibitor. In some embodiments, the therapeutic compound comprises a JAK inhibitor that inhibits JAK1, JAK3, JAK1 and JAK3, or JAK1, JAK2, and JAK3. In some embodiments, the therapeutic compound comprises a JAK inhibitor selected from the group consisting of ruxolitinib, tofacitinib, oclacitinib, baricitinib, filgotinib, gandotinib, lestaurtinib, momelotinib, pacritinib, PF-04965842, upadacitinib, peficitinib, fedratinib, cucurbitacin I, decernotinib, INCB018424, AC430, BMS-0911543, GSK2586184, VX-509, R348, AZD1480, CHZ868, PF-956980, AG490, WP-1034, JAK3 inhibitor IV, atiprimod, FM-381, SAR20347, AZD4205, ARN4079, NIBR-3049, PRN371, PF-06651600, JAK3i, JAK3 inhibitor 31, PF-06700841, NC1153, EP009, Gingerenone A, JANEX-1, cercosporamide, JAK3-IN-2, PF-956980, Tyk2-IN-30, Tyk2-IN-2, JAK3-IN1, WHI-P97, TG-101209, AZ960, NVP-BSK805, NSC 42834, FLLL32, SD 1029, WIH-P154, WHI-P154, TCS21311, JAK3-IN-1, JAK3-IN-6, JAK3-IN-7, XL019, MS-1020, AZD1418, WP1066, CEP33779, ZM 449829, SHR0302, JAK1-IN-31, WYE-151650, EXEL-8232, solcitinib, itacitinib, cerdulatinib, PF-06263276, delgotinib, AS2553627, JAK-IN-35, ASN-002, AT9283, diosgenin, JAK inhibitor 1, JAK-IN-1, LFM-A13, NS-018, RGB-286638, SB1317, curcumol, Go6976, JAK2 inhibitor G5-7, myricetin, and pyridine 6. In some embodiments, the methods further comprise adjusting the pH of the aqueous continuous phase into which the feed is dispersed in step (d). In some embodiments, the pH of the aqueous continuous phase is adjusted with a buffer solution selected from the group consisting of glycine, glycyl-glycine, tricine, HEPES, MOPS, sulfonate, ammonia, potassium phosphate, CHES, borate, TAPS, Tris, bicine, TAPSO, TES, and Tris buffer solutions. In some embodiments, the pH of the aqueous continuous phase is adjusted with a buffer solution selected from the group consisting of glycylglycine, bicine, and tricine. In some embodiments, the pH of the aqueous continuous phase is adjusted to the first pI minus 0.5 or greater. In some embodiments, the pH of the aqueous continuous phase is adjusted to about 8 to about 9.5. In some embodiments, the pH of the aqueous continuous phase is adjusted to about 9. In some embodiments, the pH of the aqueous continuous phase is adjusted to about 7.5 to about 8.5. In some embodiments, the pH of the aqueous continuous phase is adjusted to about 8. In some embodiments, the droplet is allowed to harden in (e) for at least about 120 minutes. In some embodiments, the methods further comprise, after step (d) and prior to step (e), washing the microsphere in a second aqueous continuous phase. In some embodiments, the methods further comprise, after washing the microsphere in a second aqueous continuous phase, performing size-selective filtration on the microsphere. In some embodiments, the second aqueous continuous phase has the same composition as the first aqueous continuous phase. In some embodiments, the methods further comprise, after step (e), washing the microsphere in an aqueous alcohol solution. In some embodiments, the aqueous alcohol solution comprises an aliphatic alcohol at a concentration of between about 1% and about 20%. In some embodiments, the aliphatic alcohol is ethanol or isopropanol. In some embodiments, the aqueous alcohol solution comprises ethanol at a concentration of about 10%. In some embodiments, the aqueous alcohol solution further comprises a buffer. In some embodiments, the buffer is an acetate buffer. In some embodiments, the aqueous alcohol solution is buffered to a pH of less than about 7, less than about 6, or less than about 5, e.g., a pH of about 4. In some embodiments, the methods further comprise, after step (e), lyophilizing the microsphere. In some embodiments, the methods further comprise, after step (e), spray drying the microsphere. In some embodiments, the feed of step (c) comprises a ratio of between 10:1 and 10:3 (first polymer:therapeutic compound or salt) by weight. In some embodiments, the feed of step (c) comprises the therapeutic compound or salt at a concentration of between about 10 mg/mL and about 60 mg/mL by weight. In some embodiments, hardening the droplet in step (e) comprises exacervation. In some embodiments, the first solvent comprises ethanol, propanol, or methanol. In some embodiments, the second solvent comprises dichloromethane (DCM), chloroform, or ethyl acetate. In some embodiments, dispersing the feed in step (d) comprises use of a membrane. In some embodiments, the membrane comprises a material treated to increase hydrophilicity of the membrane. In some embodiments, the membrane is coated with a hydrophilic polymer. In some embodiments, the membrane comprises stainless steel, tantalum, tungsten, molybdenum, manganese, tin, zinc, or an alloy thereof. In some embodiments, the membrane comprises porous glass or a ceramic. In some embodiments, the membrane comprises pores having a size from about 5 μm to about 50 μm. In some embodiments, the membrane comprises pores having a size from about 5 μm to about 20 μm. In some embodiments, the membrane comprises pores having a size from about 5 μm to about 50 μm, and wherein the feed is dispersed in step (d) at a flow rate of about 130 nL/min/pore. In some embodiments, the feed is dispersed in step (d) at a flow rate of between about 0.1 nLmin⁻¹ μm⁻² (pore size) and about 1 nLmin⁻¹ μm⁻² (pore size). In some embodiments, the flow rate of the continuous phase is about 1.5 L/min to about 3.5 L/min or about 1.7 L/min to about 3.4 L/min, e.g., about 3.4 L/min. In some embodiments, the flow rate of the dispersed phase is about 8 mL/min to about 13 mL/min or about 9 mL/min to about 12 mL/min, e.g., about 10 mL/min. In some embodiments, the feed is dispersed in step (d) by applying shear force. In some embodiments, the shear force is between about 1,900 s⁻¹ and about 190,000 s^{-s} or between about 500 s⁻¹ and about 40,000 s⁻¹. In some embodiments, the aqueous continuous phase further comprises a surfactant. In some embodiments, the surfactant is selected from the group consisting of polysorbate 20, polysorbate 80, poloxamer, and polyvinyl alcohol (PVA). In some embodiments, the concentration of the surfactant in the aqueous continuous phase is from 0.05% to 1% (w/w). In some embodiments, the concentration of the surfactant in the aqueous continuous phase is about 0.5% (w/w). In some embodiments, the microsphere is substantially free of small hydrocarbons (e.g., C₁-C₁₆ hydrocarbons, C₁-C₁₆ alkanes, heptane) and/or silicon oil. In some embodiments, the methods do not comprise the addition of a small hydrocarbon and/or silicon oil.

Further provided herein is a microsphere produced by a method according to any one of the above embodiments.

Further provided herein is a pharmaceutical composition comprising the microsphere of any one of the above embodiments.

Further provided herein are methods of treating a condition, comprising: administering to the individual a therapeutically effective amount of the microsphere of any one of the above embodiments or the composition of any one of the above embodiments, wherein the condition is selected from the group consisting of acromegaly, carcinoid tumors, vasoactive intestinal peptide secreting tumors, diarrhea associated with acquired immune deficiency syndrome (AIDS), diarrhea associated with chemotherapy, diarrhea associated with radiation therapy, dumping syndrome, adrenal gland neuroendocrine tumors, bowel obstruction, enterocutaneous fistulae, gastrinoma, acute bleeding of gastroesophageal varices, islet cell tumors, lung neuroendocrine tumors, malignancy, meningiomas, gastrointestinal tract neuroendocrine tumors, thymus neuroendocrine tumors, pancreatic fistulas, pancreas neuroendocrine tumors, pituitary adenomas, short-bowel syndrome, small or large cell neuroendocrine tumors, thymomas and thymic carcinomas, Zollinger Ellison syndrome, acute pancreatitis, breast cancer, chylothorax, congenital lymphedema, diabetes mellitus, gastric paresis, hepatocellular carcinoma, non-variceal upper gastrointestinal bleeding, obestity, pancreaticoduodenectomy, prostate cancer, protein-losing enteropathy, small cell lung cancer, thyroid cancer, thyroid eye disease, vascular (arterio-venous) malformations of the gastrointestinal tract, polycystic kidney disease, Cushing's disease, GHRH-producing tumors, and other conditions resulting in abnormally elevated growth hormone, insulin, or glucagon levels in an individual in need thereof. In some embodiments, the individual is a human.

Further provided herein are methods of treating growth hormone deficiency, comprising: administering to an individual in need thereof a therapeutically effective amount of the microsphere of any one of the above embodiments or the composition of any one of the above embodiments. In some embodiments, the individual is a human.

In some embodiments of any of the above embodiments, the microsphere or composition is administered to the individual by injection. In some embodiments, the injection is a subcutaneous or intramuscular injection.

Further provided herein are methods of treating alopecia, comprising: administering to an individual in need thereof a therapeutically effective amount of the microsphere of any one of the above embodiments or the composition of any one of the above embodiments, wherein the therapeutic compound or pharmaceutically acceptable salt thereof is a JAK inhibitor. In some embodiments, the therapeutic compound comprises a JAK inhibitor that inhibits JAK1, JAK3, JAK1 and JAK3, or JAK1, JAK2, and JAK3. In some embodiments, the therapeutic compound comprises a JAK inhibitor selected from the group consisting of ruxolitinib, tofacitinib, oclacitinib, baricitinib, filgotinib, gandotinib, lestaurtinib, momelotinib, pacritinib, PF-04965842, upadacitinib, peficitinib, fedratinib, cucurbitacin I, decernotinib, INCB018424, AC430, BMS-0911543, GSK2586184, VX-509, R348, AZD1480, CHZ868, PF-956980, AG490, WP-1034, JAK3 inhibitor IV, atiprimod, FM-381, SAR20347, AZD4205, ARN4079, NIBR-3049, PRN371, PF-06651600, JAK3i, JAK3 inhibitor 31, PF-06700841, NC1153, EP009, Gingerenone A, JANEX-1, cercosporamide, JAK3-IN-2, PF-956980, Tyk2-IN-30, Tyk2-IN-2, JAK3-IN1, WHI-P97, TG-101209, AZ960, NVP-BSK805, NSC 42834, FLLL32, SD 1029, WIH-P154, WHI-P154, TCS21311, JAK3-IN-1, JAK3-IN-6, JAK3-IN-7, XL019, MS-1020, AZD1418, WP1066, CEP33779, ZM 449829, SHR0302, JAK1-IN-31, WYE-151650, EXEL-8232, solcitinib, itacitinib, cerdulatinib, PF-06263276, delgotinib, AS2553627, JAK-IN-35, ASN-002, AT9283, diosgenin, JAK inhibitor 1, JAK-IN-1, LFM-A13, NS-018, RGB-286638, SB1317, curcumol, Go6976, JAK2 inhibitor G5-7, myricetin, and pyridine 6. In some embodiments, the microsphere or composition is administered to the individual by dermal or subdermal injection. In some embodiments, the individual is a human.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1A shows the chemical structures of the exemplary therapeutic compound octreotide (major hydrophobic and hydrophilic groups are labeled) and the exemplary polymer poly(lactic-co-glycolic acid), PLGA.

FIG. 1B shows the chemical structures of exemplary somatostatins. * indicates site of a bridge between each formula and the next formula shown.

FIG. 2 illustrates the features of a double emulsion, oil/water/oil microsphere that incorporates a hydrophilic therapeutic compound in a hydrophobic matrix (e.g., made using PLGA), such as SANDOSTATIN® LAR depot (octreotide acetate).

FIG. 3 shows the structure of a SANDOSTATIN® LAR depot (octreotide acetate) microsphere, as imaged by scanning electron microscopy (SEM). Surface (left) and interior (right) views are shown.

FIG. 4A shows an SEM image of the surface of a SANDOSTATIN® LAR depot (octreotide acetate) formulation. Scale bar indicates 50 μm.

FIG. 4B shows the size distribution of microspheres from two samples of a SANDOSTATIN® LAR depot formulation. Graph indicates frequency (Gaussian fit) as a function of microsphere diameter (μm).

FIG. 5 illustrates an improved process for generating microspheres with a therapeutic compound and polymer(s), in accordance with some embodiments.

FIG. 6A illustrates the interactions between a polymer having an anionic C-terminus (e.g., PLGA) and a therapeutic compound with positive charges (e.g., octreotide).

FIG. 6B shows the interactions between PLGA and 20 mg/mL octreotide (right), as compared with PLGA alone.

FIG. 6C shows the combinations of solvent and cosolvent tested for generating octreotide microspheres.

FIG. 6D shows the PLGA polymers tested for generating octreotide microspheres.

FIG. 7 shows a plurality of octreotide/PLGA microspheres formulated according to formulation 149, as imaged by SEM. Scale bar indicates 200 μm.

FIG. 8 shows multiple SEM images of a plurality of octreotide/PLGA microspheres formulated according to formulation 149, as compared to a plurality of SANDOSTATIN® LAR microspheres. Shown are: formulation 149 with scale bar indicating 200 μm (top left); formulation 149 with scale bar indicating 50 μm (top right); formulation 149 with scale bar indicating 20 μm (bottom left); and SANDOSTATIN® LAR with scale bar indicating 50 μm (bottom right).

FIG. 9 shows another set of SEM images of a plurality of octreotide/PLGA microspheres formulated according to formulation 149. Shown are: formulation 149 with scale bar indicating 200 μm (top left); formulation 149 with scale bar indicating 20 μm (top right); and formulation 149 with scale bar indicating 10 μm (bottom left).

FIG. 10 shows multiple SEM cross-sections of octreotide/PLGA microspheres formulated according to formulation 149. Scale bars indicate 10 μm (top left, bottom left, bottom right) or 5 μm (top right).

FIG. 11 shows multiple SEM images of a plurality of octreotide/PLGA microspheres formulated according to formulation 131. Shown are: formulation 131.3R with scale bar indicating 200 μm (top left); formulation 131 with scale bar indicating 50 μm (top right); and formulation 131 with scale bar indicating 20 μm (bottom left).

FIG. 12 shows another set of SEM images of a plurality of octreotide/PLGA microspheres formulated according to formulation 131. Shown are: formulation 131 with scale bar indicating 200 μm (top left); formulation 131 with scale bar indicating 50 μm (top right); and formulation 131 with scale bar indicating 10 μm (bottom left).

FIG. 13 shows multiple SEM cross-sections of octreotide/PLGA microspheres formulated according to formulation 131. Scale bars indicate 10 μm.

FIG. 14 shows multiple SEM images of a plurality of octreotide/PLGA microspheres formulated according to formulation 73. Shown are: formulation 73 with scale bar indicating 200 μm (top left); formulation 73 with scale bar indicating 50 μm (top right); and formulation 73 with scale bar indicating 20 μm (bottom left).

FIG. 15 shows another set of SEM images of a plurality of octreotide/PLGA microspheres formulated according to formulation 73. Shown are: formulation 73 with scale bar indicating 200 μm (top left); formulation 73 with scale bar indicating 20 μm (top right); and formulation 73 with scale bar indicating 10 μm (bottom left).

FIG. 16 shows multiple SEM cross-sections of octreotide/PLGA microspheres formulated according to formulation 73. Scale bars indicate 10 μm (top right, bottom left, bottom right) or 20 μm (top left).

FIG. 17A compares the size distributions of SANDOSTATIN® LAR microspheres (batch 1: dotted line; batch 2: solid black line) with the size distribution of a plurality of octreotide/PLGA microspheres formulated according to formulation 149 (blue).

FIG. 17B compares the size distributions of SANDOSTATIN® LAR microspheres (batch 1: dotted line; batch 2: solid black line) with the size distributions of a plurality of octreotide/PLGA microspheres prepared by the methods of the present disclosure using membranes with 10 μm (green), 15 μm (yellow), or 20 μm (red) pore size.

FIG. 17C compares the cumulative size distributions of SANDOSTATIN® LAR microspheres (dotted line) with the size distributions of a plurality of octreotide/PLGA microspheres prepared by the methods of the present disclosure using membranes with 10 μm (red), 15 μm (blue), or 20 μm (green) pore size. Cumulative size (as percentage of microspheres) is plotted as a function of microsphere diameter (in μm).

FIG. 18A shows the serum concentration over time of octreotide (ng/mL) after a single subcutaneous injection of 87 mg SANDOSTATIN® LAR (4.6 mg peptide) in rabbits. Results from three injections are shown.

FIG. 18B shows the serum concentration over time of octreotide (ng/mL) after a single subcutaneous injection of 100 mg octreotide/PLGA microsphere formulation 73 (5 mg peptide) in rabbits. Results from three injections are shown.

FIG. 19A shows the serum concentration over time of octreotide (ng/mL) after a single intramuscular injection of 87 mg SANDOSTATIN® LAR (4.6 mg peptide) in rabbits. Results from three injections are shown.

FIG. 19B shows the serum concentration over time of octreotide (ng/mL) after a single intramuscular injection of octreotide/PLGA microsphere formulation 73 in rabbits. Results from three injections are shown.

FIG. 20 shows the burst Cmax (time zero to 3 hours; orange) and burst AUC (time zero to 3 hours; green) of formulations 73, 139, 137, and 121. Burst Cmax and burst AUC are normalized to 1 mg of injected octreotide for each formulation.

FIG. 21 shows that loading of octreotide in microspheres increases with increased PLGA (MW: 7 kD-17 kD) composition in microspheres formulated by blending multiple PLGA species. Octreotide loading is shown as a function of the percentage of PLGA (MW: 7 kD-17 kD) species.

FIG. 22A shows an SEM image of the surface of octreotide/PLGA microspheres produced without glycerol according to formulation 73. Arrows point to surface pores. Scale bar indicates 10 μm. Image is an enlarged version of corresponding image shown in FIG. 15.

FIG. 22B shows an SEM image of the surface of octreotide/PLGA microspheres produced with glycerol according to formulation 131. Scale bar indicates 10 μm.

FIG. 23A shows an SEM cross-section of the interior of octreotide/PLGA microspheres produced without glycerol according to formulation 73. Scale bar indicates 20 μm.

FIG. 23B shows an SEM cross-section of the interior of octreotide/PLGA microspheres produced with glycerol according to formulation 131. Scale bar indicates 10 μm.

FIG. 24 shows the burst of octreotide over time in rabbits administered an intramuscular injection of SANDOSTATIN® LAR depot (green), octreotide/PLGA microsphere formulation 73 without glycerol (red), or octreotide/PLGA microsphere formulation 73 with final concentration of 0.9 mg/mL glycerol (purple).

FIG. 25 shows degradation of octreotide in SANDOSTATIN® LAR, formulation 73, formulation 121, formulation 131 (formulation 73 with glycerol), or formulation 132 (formulation 121 with glycerol) after 2 months at 40° C. Degradation is shown as the percentage of degradation products (as percentage of main peak).

FIG. 26A shows plasma concentrations of octreotide over time in rabbits administered an intramuscular injection of 87 mg SANDOSTATIN® LAR depot (squares) or 100 mg formulation 137 (circles).

FIG. 26B shows plasma concentrations of octreotide over time in rabbits administered an intramuscular injection of 87 mg SANDOSTATIN® LAR depot (triangles) or 100 mg formulation 137 (squares). In this plot, the SANDOSTATIN® LAR depot data has been scaled to be equivalent to 100 mg injected (e.g., scaled by 100/87).

FIG. 27A shows the average plasma concentrations of octreotide over time in two minipigs administered an intramuscular injection of 1.2 mg/kg SANDOSTATIN® LAR depot (dotted line) or a subcutaneous injection of 1.44 mg/kg formulation 175 (solid line).

FIG. 27B shows the average plasma concentrations of octreotide per 1 mg of injected peptide within 6 hours post-dose in two minipigs administered an intramuscular injection of 1.2 mg/kg SANDOSTATIN® LAR depot (dotted line) or a subcutaneous injection of 1.44 mg/kg formulation 175 (solid line).

FIG. 27C shows the average plasma concentrations of octreotide per 100 mg of injected PLGA over time in two minipigs administered an intramuscular injection of 1.2 mg/kg SANDOSTATIN® LAR depot (dotted line) or a subcutaneous injection of 1.44 mg/kg formulation 175 (solid line).

FIG. 28 shows plasma concentrations of octreotide over time in rabbits administered an intramuscular injection of 87 mg SANDOSTATIN® LAR depot (red circles, dotted line), a subcutaneous injection of 100 mg formulation 175 (blue circles, solid line), or a subcutaneous injection of 100 mg formulation 173 (yellow circles, solid line).

FIG. 29A shows a set of SEM images of a plurality of octreotide/PLGA microspheres formulated using a continuous phase with glyclglycine buffer. Shown are: formulation with scale bar indicating 200 μm (top left); formulation with scale bar indicating 50 μm (top right); and formulation with scale bar indicating 10 μm (bottom left).

FIG. 29B shows multiple SEM cross-sections of octreotide/PLGA microspheres formulated using a continuous phase with glyclglycine buffer. Scale bars indicate 10 μm.

FIG. 30A shows a set of SEM images of a plurality of octreotide/PLGA microspheres formulated using a continuous phase with Tris buffer. Shown are: formulation with scale bar indicating 200 μm (top left); formulation with scale bar indicating 50 μm (top right); and formulation with scale bar indicating 10 μm (bottom left). Arrows in upper right indicate microspheres with higher surface porosity.

FIG. 30B shows multiple SEM cross-sections of octreotide/PLGA microspheres formulated using a continuous phase with Tris buffer. Scale bars indicate 20 μm (left) or 10 μm (right).

FIG. 31 shows a graph of product-related impurities generated during microsphere hardening at different pH values.

FIG. 32 shows a graph of octreotide loading after 0 or 120 minutes in solution after extrusion at the indicated pH of the hardening solution.

FIG. 33 shows DCM levels after varies times in the hardening solution.

FIG. 34 shows the microsphere size distribution from two 10 g and one 30 g batches of production.

FIG. 35 shows the microsphere size distribution from 1 g, 10 g, and 30 g batches of microspheres.

FIG. 36 shows a diagram of various microspheres formed by varying dispersed phase (DP) and continuous phase (CP) flow rates. (A) DP 10 mL/min, CP 3.4 L/min; (B) DP 10 mL/min, CP 3.0 L/min; (C) DP 9 mL/min, CP 2.3 L/min; (D) DP 9 mL/min, CP 1.7 L/min; (E) DP 12 mL/min, CP 1.7 L/min.

### DETAILED DESCRIPTION

Provided herein are microspheres (e.g., single emulsion microspheres) comprising a therapeutic compound or pharmaceutically acceptable salt thereof and one or more polymers, as well as methods of manufacture, methods of use, and kits or articles of manufacture related thereto. The present disclosure is based, at least in part, on the finding that combinations of polymers and therapeutic compounds (e.g., a therapeutic compound or salt having a pI at least 1.5 units greater than the pI of the polymer) allow for formation of single emulsion microspheres, such as for hydrophilic (e.g., charged, cationic) therapeutic compounds or salts, and also allow for increased loading of the compound or salt into microspheres containing the compound or salt and the polymer (e.g., as compared to double emulsion microspheres). Importantly, synergistic interactions during the manufacturing process between the polymer (e.g., by means of one or more anionic groups) and the therapeutic compound or salt are thought to enable a higher concentration of polymer in the feed (e.g., a homogeneous solution feed) by virtue of a cationic therapeutic compound or salt.

Moreover, the present disclosure demonstrates the surprising finding that blending multiple polymers (e.g., with different molecular weights) into these microspheres allows increased loading of the therapeutic compound or salt into the microspheres while keeping the serum pharmacokinetic burst of the microsphere constant or even reducing the burst, relative to the amount of therapeutic compound administered. For example, polymer blends that increase the concentration of anionic end group of one or more of the polymers (e.g., by virtue of addition of the lower molecular weight polymer species) result in increased solubility of the cationic compound or salt in the feed/microsphere. This allows for administration of lower volumes of drug formulation (and hence easier and less painful injections) while delivering the same amount of therapeutic compound or salt and retaining similar pharmacokinetic properties (e.g., pharmacokinetic burst).

The present disclosure is also based, at least in part, on the surprising finding that addition of a polyol (e.g., glycerol) into the microspheres reduces degradation of the therapeutic compound or salt, improves dry powder flow and reduces aggregation by providing a smoother exterior surface, and also reduces the pharmacokinetic burst of the therapeutic compound or salt in serum. These advances improve the pharmacokinetic properties and stability of the microspheres described herein.

### I. Microspheres

Certain aspects of the present disclosure relate to microspheres (e.g., single emulsion microspheres) comprising a therapeutic compound or pharmaceutically acceptable salt thereof and one or more polymers. In some embodiments, the polymer has a pI at least 1, at least 1.5, at least 2, or at least 2.5 units lower than the pI of the therapeutic compound or salt. In some embodiments, the polymer has a pI at least 1.5 units lower than the pI of the therapeutic compound or salt.

The terms microsphere denotes the encapsulation of the therapeutic compounds/peptides by the polymer, in some embodiments with the therapeutic compound/peptide distributed throughout the polymer, which is then a matrix for the therapeutic compound/peptide.

In some embodiments, a therapeutic compound or salt of the present disclosure comprises at least one cationic moiety.

In some embodiments, a therapeutic compound or salt of the present disclosure comprises a therapeutic peptide. In some embodiments, the therapeutic peptide comprises at least two amino-containing amino acid side chains. In some embodiments, the therapeutic peptide has a length from 6 to 40 amino acids, e.g., a length of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids. In certain embodiments, the therapeutic peptide has a length of 8 amino acids. In some embodiments, the therapeutic peptide is cyclic. In some embodiments, the therapeutic peptide is selected from veldoreotide, somatostatin (SST-28), SST-14, lanreotide, octreotide, vapreotide, pasireotide, and pharmaceutically acceptable salts of any of the foregoing. In some embodiments, the therapeutic peptide is human growth hormone or a pharmaceutically acceptable salt thereof. In some embodiments, the therapeutic peptide is octreotide or a pharmaceutically acceptable salt thereof.

In some embodiments, the therapeutic peptide is a somatostatin analog or a pharmaceutically acceptable salt thereof. Naturally occurring somatostatin is produced by the hypothalamus as well as other organs, e.g. the gastrointestinal tract, and mediates, together with growth-hormone releasing factor (GRF), the neuroregulation of pituitary growth hormone release. In addition to inhibition of growth hormone (GH) release by the pituitary, somatostatin is a potent inhibitor of a number of systems, including central and peripheral neural, gastrointestinal and vascular smooth muscle. It also inhibits the release of insulin and glucagon. Analogs (e.g., agonist analogs) of somatostatin are thus useful in replacing natural somatostatin in its effect on regulation of physiologic functions. For exemplary descriptions of somatostatin analogs, see, e.g., U.S. Pat. No. 5,639,480. Naturally occurring somatostatin is a tetradecapeptide having the structure:

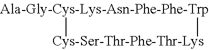

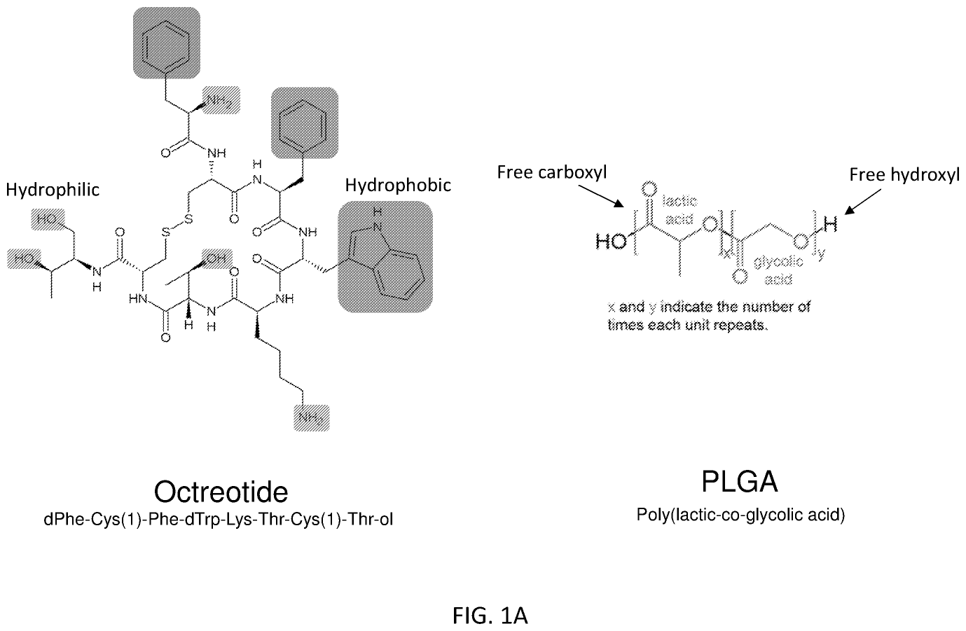

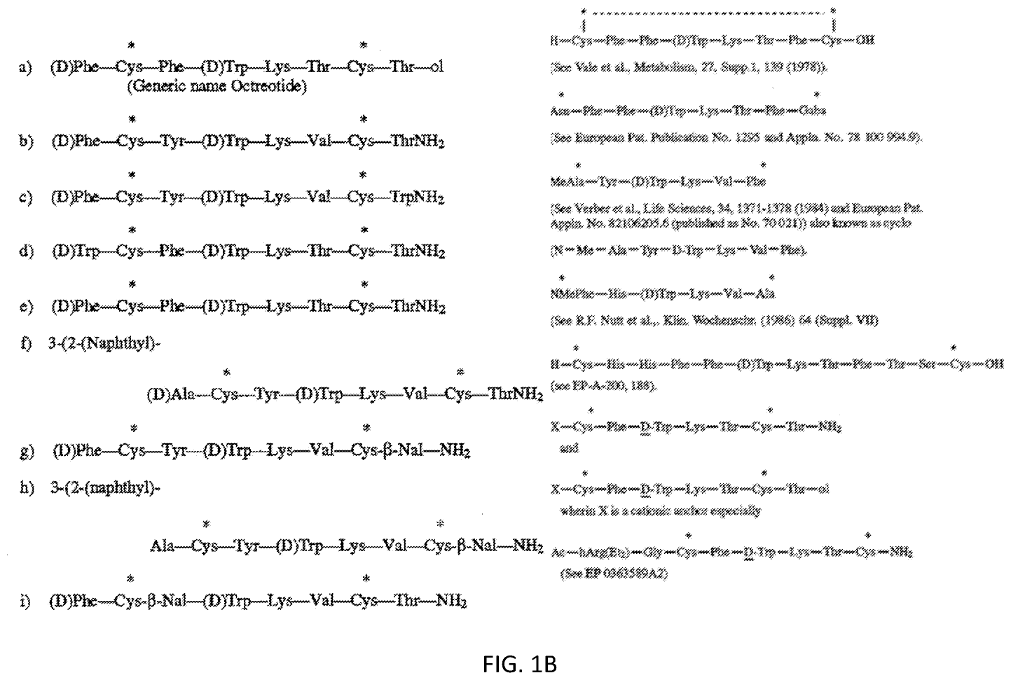

As used herein, the term “somatostatin” includes its analogues or derivatives thereof. By derivatives and analogues is understood straight-chain, bridged or cyclic polypeptides wherein one or more amino acid units have been omitted and/or replaced by one or more other amino radical(s) of and/or wherein one or more functional groups have been replaced by one or more other functional groups and/or one or more groups have been replaced by one or several other isosteric groups. In general, the term covers all modified derivatives of a biologically active peptide which exhibit a qualitatively similar effect to that of the unmodified somatostatin peptide. Somatostatins include, without limitation, those depicted in FIG. 1B.

The term derivative includes also the corresponding derivatives bearing a sugar residue. When somatostatins bear a sugar residue, this is can be coupled to an N-terminal amino group and/or to at least one amino group present in a peptide side chain, such as to a N-terminal amino group. Such compounds and their preparation are disclosed, e.g. in WO 88/02756. Exemplary derivatives are N.sup.α-[α-glucosyl-(1-4-deoxyfructosyl]-DPhe-Cys-Phe-DTrp-Lys-Thr-Cys-Thr-ol and N.sup.α-[β-deoxyfructosyl-DPhe-Cys-Phe-DTrp-Lys-Thr-Cys-Thr-ol, each having a bridge between the -Cys- moieties, optionally in acetate salt form and described in Examples 2 and 1 respectively of the above mentioned application.

In some embodiments, the therapeutic peptide is selected from somatostatin (SST-28), SST-14, lanreotide, octreotide, vapreotide, pasireotide, and pharmaceutically acceptable salts of any of the foregoing. Octreotide derivatives are also contemplated for use and include, without limitation, those comprising the moiety:

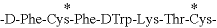

having a bridge between Cys residues.

The somatostatins may exist e.g. in free form, salt form or in the form of complexes thereof. Acid addition salts may be formed with e.g. organic acids, polymeric acids and inorganic acids. Acid addition salts include e.g. the hydrochloride and acetates. Complexes are e.g. formed from somatostatins on addition of inorganic substances, e.g. inorganic salts or hydroxides such as Ca- and Zn-salts and/or an addition of polymeric organic substances.

The acetate salt is an exemplary salt for such formulations, especially for microspheres leading to a reduced initial drug burst. The present disclosure also provides the pamoate salt, which is useful, particularly for implants and the process for its preparation. The pamoate may be obtained in conventional manner, e.g. by reacting embonic acid (pamoic acid) with octreotide e.g. in free base form. The reaction may be effected in a polar solvent, e.g. at or below room temperature.

In some embodiments, a therapeutic compound or salt of the present disclosure comprises a small molecule drug or compound.

In some embodiments, the therapeutic compound or salt comprises an mTOR inhibitor. The term “mTOR inhibitor” broadly encompasses multiple classes of molecules, including molecules that bind FKBP12 (e.g., first-generation mTOR inhibitors such as rapamycin and rapalogs that inhibit mTORC1), molecules that inhibit the kinase activity of mTOR (e.g., second-generation, ATP-competitive mTOR inhibitors that inhibit mTORC1 and mTORC2), molecules that bind FKBP12 and inhibit the kinase activity of mTOR (e.g., third-generation mTOR inhibitors such as RapaLinks), and dual PI3K/mTOR inhibitors (e.g., BEZ235 or LY3023414). In some embodiments, an mTOR inhibitor inhibits mTORC1, mTORC2, or both. Examples of specific mTOR inhibitors include, without limitation, tacrolimus (also known as FK506, fujimycin, PROGRAF®, PROTOPIC®, ADVAGRAF®, ENVARSUS®, and ASTAGRAF®), temsirolimus (also known as CCI-779 and TORISEL®), everolimus (also know n as RAD001, ZORTRESS®, AFINITOR®, CERTICAN®, VOTUBIA®, and Evertor), rapamycin (also known as sirolimus and RAPAMUNE®), ridaforolimus (also known as AP23573, MK-8669, and deforolimus), AZD8055, Ku-0063794, PP242, PP30, Torinl, WYE-354, PI-103, BEZ235 (also known as NVP-BEZ235 and dactolisib), PKI-179 (also known as PKI-587), LY3023414, omipalisib (also known as GSK2126458 and GSK458), sapanisertib (also known as MLN0128 and INK128), OSI-027, RapaLink-land voxtalisib (also known as XL765 and SAR245409).

In some embodiments, the therapeutic compound or salt comprises a glucocorticoid, e.g., a compound that binds the glucocorticoid receptor. Examples of specific glucocorticoids include, without limitation, triamcinolone (e.g., triamcinolone acetonide), beclomethasone, betamethasone, budesonide, cortisone, hydrocortisone, methylprednisolone, prednisolone, prednisone, and dexamethasone.

In some embodiments, the therapeutic compound or salt comprises a Janus kinase (JAK) inhibitor. The term “JAK inhibitor” broadly encompasses molecules that inhibit the function of one or more JAK family kinases, such as JAK1, JAK2, JAK3, and TYK2. For example, in some embodiments, a JAK inhibitor inhibits one or more activities of JAK1; JAK2; JAK3; JAK1 and JAK2; JAK1 and JAK3; JAK3 and JAK2; TYK2 and JAK1; TYK2 and JAK2; TYK2 and JAK3; JAK1, JAK2, and JAK3; or JAK1, JAK2, TYK2, and JAK3. Examples of specific JAK inhibitors include, without limitation, ruxolitinib (also known as JAKAFI®, JAKAVI®, and INCB018424, including the phosphate and sulfate salts and S enantiomer), tofacitinib (also known as tasocitinib, CP-690550, XELJANZ® and JAKVINUS®, including (3R,4S), (3S,4R), and (3S,4S) enantiomers and the citrate salt), oclacitinib (also known as APOQUEL®, including the maleate salt), baricitinib (also known as LY3009104, INCB-28050, and OLUMIANT®, including the phosphate salt), filgotinib (also known as G-146034 and GLPG-0634), gandotinib (also known as LY-2784544), lestaurtinib (also known as CEP-701), momelotinib (also known as GS-0387 and CYT-387, including mesylate and sulfate salts), pacritinib (also known as SB1518), PF-04965842, upadacitinib (also known as ABT-494), peficitinib (also known as ASP015K and JNJ-54781532), fedratinib (also known as SAR302503 and TG101348), cucurbitacin I (also known as JSI-124), decernotinib (also known as VX-509 and VRT-831509), INCB018424, AC430, BMS-0911543, GSK2586184, VX-509, R348, AZD1480, CHZ868, PF-956980, AG490, WP-1034, JAK3 inhibitor IV (also known as ZM-39923, including the hydrochloride salt), atiprimod (including the dihydrochloride salt), FM-381, SAR20347, AZD4205, ARN4079, NIBR-3049, PRN371, PF-06651600 (including the malonate salt), JAK3i, JAK3 inhibitor 31, PF-06700841 (including the tosylate salt), NC1153, EP009, Gingerenone A, JANEX-1 (also known as WHI-P131), cercosporamide, JAK3-IN-2, PF-956980, Tyk2-IN-30, Tyk2-IN-2, JAK3-IN1, WHI-P97, TG-101209, AZ960, NVP-BSK805 (including the dihydrochloride salt), NSC 42834 (also known as Z3), FLLL32, SD 1029, WIH-P154, WHI-P154, TCS21311, JAK3-IN-1, JAK3-IN-6, JAK3-IN-7, XL019, MS-1020, AZD1418, WP1066, CEP33779, ZM 449829, SHR0302, JAK1-IN-31, WYE-151650, EXEL-8232, solcitinib (also known as GSK-2586184 and GLPG-0778), itacitinib (also known as INCB039110, including the adipate salt), cerdulatinib (also known as PRT062070 and PRT2070), PF-06263276, delgotinib (also known as JTE-052), AS2553627, JAK-IN-35, ASN-002, AT9283, diosgenin, JAK inhibitor 1 (see US20170121327, compound example 283), JAK-IN-1, LFM-A13, NS-018 (including hydrochloride and maleate salts), RGB-286638, SB1317 (also known as TG02), curcumol, Go6976, JAK2 inhibitor G5-7, myricetin (also known as NSC 407290 and cannabiscetin), and pyridine 6 (also known as CMP6). For more description and chemical structures of exemplary JAK inhibitors, see, e.g., U.S. Pat. Nos. 9,198,911; 9,763,866; 9,737,469; 9,730,877; 9,895,301; 9,249,149; 9,518,027; 9,776,973; 9,549,367; and 9,931,343.

In some embodiments, a microsphere of the present disclosure comprises more than one therapeutic compound or pharmaceutically acceptable salt thereof. For example, a microsphere of the present disclosure can comprise multiple somatostatins, e.g., to target a particular somatostatin receptor profile in order to attain an altered pharmacodynamics effect.

In some embodiments, a polymer of the present disclosure comprises at least one anionic terminus. In some embodiments, a polymer of the present disclosure comprises at least one acid terminus. In certain embodiments, a polymer of the present disclosure comprises at least one carboxylic acid terminus. Exemplary polymers include, without limitation, those comprising poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer.

Exemplary polymers can also include those prepared from biocompatible and biodegradable polymers, such as linear polyesters, branched polyesters which are linear chains radiating from a polyol moiety, e.g. glucose. Other esters are those of polylactic acid, polyglycolic acid, polyhydroxybutyric acid, polycaprolactone, polyalkylene oxalate, polyalkylene glycol esters of acids of the Kreb's cycle, e.g. citric acid cycle and the like and copolymers thereof. The linear polyesters may be prepared from the alphahydroxy carboxylic acids, e.g. lactic acid, and glycolic acid, by the condensation of the lactone dimers, see for example U.S. Pat. No. 3,773,919.

The branched polyesters may be prepared using polyhydroxy compounds e.g. polyol e.g. glucose or mannitol as the initiator. These esters of a polyol are known and described in GB 2,145,422 B. The polyol contains at least 3 hydroxy groups and has a molecular weight of up to 20 kD, with at least 1, at least 2, e.g. as a mean 3 of the hydroxy groups of the polyol being in the form of ester groups, which contain poly-lactide or co-poly-lactide chains. Typically 0.2% glucose is used to initiate polymerization. The structure of the branched polyesters may be star shaped. The polyester chains in the linear and star polymer compounds optionally used according to the present disclosure are copolymers of the alpha carboxylic acid moieties, lactic acid and glycolic acid, or of the lactone dimers. The molar ratios of lactide:glycolide is from about 5:25 to 25:75, e.g. 60:40 to 40:60, with from 55:45 to 45:55, e.g. 55:45 to 50:50. The star polymers may be prepared by reacting a polyol with a lactide and optionally also a glycolide at an elevated temperature in the presence of a catalyst, which makes a ring opening polymerization feasible.

In some embodiments, a polymer of the present disclosure comprises a molecular weight less than or equal to 17 kD. In some embodiments, a molecular weight refers to the average molecular weight of a polymer species. In some embodiments, a molecular weight refers to the minimum or maximum molecular weight of a polymer species. For example, RESOMER® RG 502H (Evonik Industries) has a molecular weight of 7 kD-17 kD, and RESOMER® RG 503H (Evonik Industries) has a molecular weight of 24 kD-38 kD. In some embodiments, a polymer of the present disclosure comprises a maximum molecular weight less than or equal to 17 kD. In some embodiments, a polymer of the present disclosure comprises a minimum molecular weight less than or equal to 7 kD. In some embodiments, a polymer of the present disclosure comprises a maximum molecular weight less than or equal to 38 kD. In some embodiments, a polymer of the present disclosure comprises a minimum molecular weight less than or equal to 24 kD.

In some embodiments, a microsphere of the present disclosure comprises (or is made with a feed comprising) more than one polymer, e.g., 2, 3, 4, 5, or more polymers. In some embodiments, at least one of the polymers has a pI at least 1.5 units lower than the pI of the therapeutic compound or salt. In some embodiments, at least one of the polymers comprises one or more anionic termini. In some embodiments, a first of the multiple polymers has a lower molecular weight than a second of the multiple polymers. In some embodiments, a first of the multiple polymers has a lower molecular weight (e.g., average, minimum, or maximum molecular weight) by at least 10 kD than a second of the multiple polymers. In some embodiments, the molecular weight (e.g., average, minimum, or maximum molecular weight) of the first polymer is less than or equal to 17 kD. In some embodiments, the molecular weight (e.g., average, minimum, or maximum molecular weight) of the first polymer is less than or equal to 17 kD, and the molecular weight (e.g., average, minimum, or maximum molecular weight) of the second polymer is at least 24 kD. In some embodiments, the first polymer has one or more anionic termini, and the second polymer does not.

In some embodiments, the first polymer comprises poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer; and the second polymer comprises a polymer independently selected from poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer. In some embodiments, the first polymer comprises poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer; and the second polymer comprises the same polymer (but a species thereof having a different molecular weight than the first polymer) of poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer. In some embodiments, the first and second polymers represent different species of PLGA. In some embodiments, the first and second polymers represent different species of PLGA that both comprise a carboxylic acid terminus. In some embodiments, the first and second polymers are PLGA species having a difference in minimum molecular weight of at least about 7 kD, at least about 10 kD, at least about 17 kD, or at least about 20 kD. In some embodiments, the first and second polymers are PLGA species having a difference in maximum molecular weight of at least about 7 kD, at least about 10 kD, at least about 17 kD, or at least about 20 kD. In some embodiments, the first and the second polymers both comprise PLGA, and the molecular weight (e.g., average, minimum, or maximum molecular weight) of the first polymer is at least 10 kD lower than the molecular weight of the second polymer. In some embodiments, a microsphere of the present disclosure comprises (or is made with a feed comprising) PLGA having a molecular weight of 7 kD-17 kD, and PLGA having a molecular weight of 24 kD-38 kD.

In some embodiments, a microsphere of the present disclosure comprises (or is made with a feed comprising) the first and the second polymer at a ratio of between about 20:80 and about 80:20 (first polymer:second polymer). In some embodiments, a microsphere of the present disclosure comprises (or is made with a feed comprising) the first and the second polymer at a ratio of greater than 20:80, 25:75, 30:70, 35:65, 40:60, 45:55, 50:50, 55:45, 60:40, 65:35, 70:30, or 75:25 (first polymer:second polymer). In some embodiments, a microsphere of the present disclosure comprises (or is made with a feed comprising) the first and the second polymer at a ratio of less than 80:20, 75:25, 70:30, 65:35, 60:40, 55:45, 50:50, 45:55, 40:60, 35:65, 30:70, or 25:75 (first polymer:second polymer). That is, a microsphere of the present disclosure comprises (or is made with a feed comprising) the first and the second polymer at a ratio having an upper limit of 80:20, 75:25, 70:30, 65:35, 60:40, 55:45, 50:50, 45:55, 40:60, 35:65, 30:70, or 25:75 and an independently selected lower limit of 20:80, 25:75, 30:70, 35:65, 40:60, 45:55, 50:50, 55:45, 60:40, 65:35, 70:30, or 75:25 (first polymer:second polymer), wherein the upper limit is greater than the lower limit. In some embodiments, a microsphere of the present disclosure comprises (or is made with a feed comprising) the first and the second polymer at a ratio of 20:80, 25:75, 30:70, 35:65, 40:60, 45:55, 50:50, 55:45, 60:40, 65:35, 70:30, 75:25, or 80:20 (first polymer:second polymer). In certain embodiments, a microsphere of the present disclosure comprises (or is made with a feed comprising) the first and the second polymer at a ratio of about 75:25 (first polymer:second polymer). In certain embodiments, a microsphere of the present disclosure comprises (or is made with a feed comprising) the first and the second polymer at a ratio of about 65:35 (first polymer:second polymer).

In some embodiments, a microsphere of the present disclosure further comprises (or is made with a feed further comprising) a polyol. In some embodiments, the polyol comprises a (C₃₋₆) carbon chain containing alcohol having 2 to 6 hydroxyl groups and a mono- or di-saccharide, an esterified polyol having at least 3 polylactide-co-glycolide chains, glycols (e.g., propylene glycol), glucose, mannitol, or glycerol. In certain embodiments, the polyol is glycerol.

In some embodiments, the therapeutic compound or salt is greater than 4%, greater than 5%, greater than 6%, greater than 7%, greater than 8%, or greater than 9% (by total weight) of the microsphere.

In some embodiments, the present disclosure provides a plurality of microspheres, where at least 50%, at least 60%, at least 70%, at least 80%, at least 85%, or at least 90% of the microspheres of the plurality have a diameter within 10 μm, within 15 μm, or within 20 μm above or below a median diameter of the plurality. In some embodiments, the median diameter of the plurality is between about 5 μm to about 100 μm. In some embodiments, the median diameter of the plurality is between about 10 μm to about 50 μm. In some embodiments, the median diameter of the plurality is between about 20 μm to about 40 μm. In some embodiments, the microspheres of the plurality have at least 10% less, at least 15% less, at least 20% less, or at least 25% less pore surface area as measured by gas absorption isotherms using N₂, CO₂, or Hg as compared to a reference microsphere. For example, in some embodiments, the microspheres of the plurality comprise a polyol (e.g., glycerol), and the microspheres of the plurality have at least 10% less, at least 15% less, at least 20% less, or at least 25% less pore surface area as measured by gas absorption isotherms using N₂, CO₂, or Hg as compared to a reference microsphere that lacks the polyol (but optionally comprises the same therapeutic compound or salt and/or polymer(s)). Exemplary methods for measurements using gas absorption isotherms are known in the art and described below. In some embodiments, specific surface area is measured by the Brunauer-Emmett-Teller (BET) theory using, e.g., gas physisorption. Increased porosity leads to higher specific surface area.

In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres are 22-36 μm in diameter. In some embodiments, the present disclosure provides a plurality of microspheres, where at least 90-95% of the microspheres are 22-36 μm in diameter. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres are 26-34 μm in diameter. In some embodiments, the present disclosure provides a plurality of microspheres, where at least 60-70% of the microspheres are 26-34 μm in diameter. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres are 20-40 μm in diameter. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres are 28-32 μm in diameter. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres are 22-34 μm in diameter. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres are 26-36 μm in diameter.

In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 7 μm of a mean diameter of 29 μm. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 7 μm of a mean diameter of 30 μm. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 4 μm of a mean diameter of 30 μm. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 4 μm of a mean diameter of 29 μm. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 10 μm of a mean diameter of 30 μm. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 2 μm of a mean diameter of 30 μm.

In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 7% of the mean diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 7% of the median diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 6% of the mean diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 6% of the median diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 5% of the mean diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 5% of the median diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 7% of the mean diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 7% of the median diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 4% of the mean diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 4% of the median diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 10% of the mean diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 10% of the median diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 15% of the mean diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 15% of the median diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 20% of the mean diameter of the plurality of microspheres. In some embodiments, the present disclosure provides a plurality of microspheres, where at least 50%, at least 55%, at least 60%, at least 65%, at least 70%, at least 75%, at least 80%, at least 85%, at least 90%, or at least 95% of the microspheres of the plurality have a diameter within (e.g., plus or minus) 20% of the median diameter of the plurality of microspheres.

Advantageously, the present disclosure provides microspheres that allow for increased loading of therapeutic compound or salt while maintaining similar pharmacokinetic properties, e.g., pharmacokinetic burst in serum. This allows the injection of lower volumes to achieve the desired drug dosage and pharmacokinetic properties, providing less painful injections for the patient. In some embodiments, a microsphere of the present disclosure induces a pharmacokinetic burst (e.g., in serum, per mg of the therapeutic compound or salt) that is equal to or less than pharmacokinetic burst (e.g., in serum, per mg of the therapeutic compound or salt) for a reference microsphere. In some embodiments, the reference microsphere comprises the therapeutic compound or salt at a lower loading level than the microsphere of the present disclosure. For example, in some embodiments, a microsphere of the present disclosure comprises a therapeutic compound or salt of the present disclosure and first and second polymers of the present disclosure (e.g., where the first polymer has a pI at least 1.5 units lower than the pI of the compound or salt and/or has a lower molecular weight than the second polymer) and induces a pharmacokinetic burst (e.g., in serum, per mg of the therapeutic compound or salt) that is equal to or less than pharmacokinetic burst (e.g., in serum, per mg of the therapeutic compound or salt) for a reference microsphere that comprises the therapeutic compound or salt and the second polymer but lacks the first polymer. As is known in the art, the release profile of therapeutic compound or salt from a microsphere formulation may follow the pattern of an initial release of compound (e.g., pharmacokinetic burst, such as that observed within time intervals including, but not limited to, 30 minutes, 1 hour, 2 hours, 3 hours, 4 hours, 5 hours, 6 hours, 12 hours, 24 hours, 36 hours, 48 hours, or 72 hours) characterized by a sharp increase in serum compound levels, followed by a longer, sustained release. The burst and sustained release can also be due to different factors. For example, the burst can be due, e.g., to surface pores or surface-absorbed compound, whereas the sustained release can originate from slow degradation of the microsphere (e.g., gradual polymer degradation). For many microsphere formulations, the sustained release, and not the burst, provides the therapeutic benefit. Accordingly, it may be desirable to limit or reduce the magnitude of the burst.

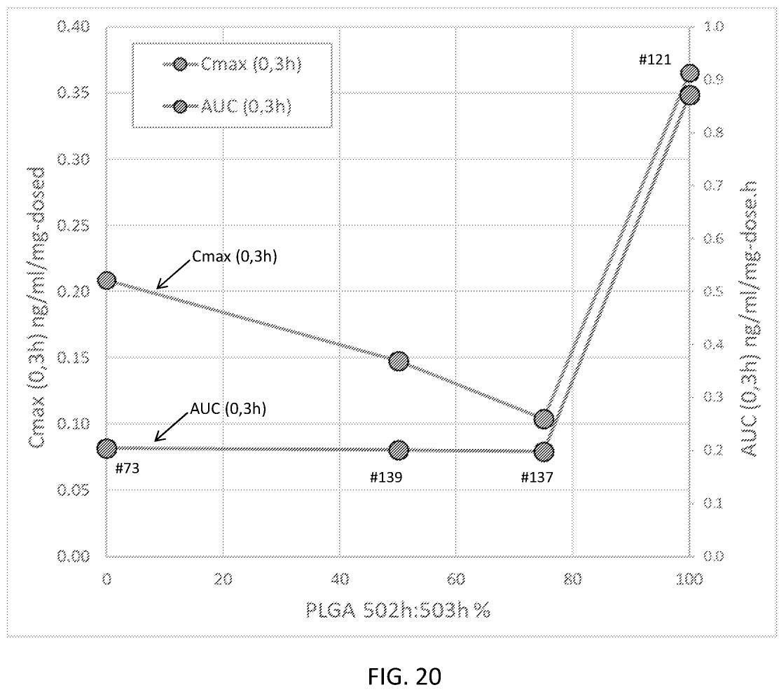

In some embodiments, a reference microsphere of the present disclosure is a double emulsion microsphere. For example, it is thought that the improved formulations described herein provide a more homogeneous feed solution that results in single emulsion microspheres with a higher loading of therapeutic compound at no burst penalty, as compared with a double emulsion microsphere. In some embodiments, a microsphere of the present disclosure (e.g., a single emulsion microsphere) allows for higher loading than a reference microsphere (e.g., a double emulsion microsphere) at equal or lesser pharmacokinetic burst normalized to amount of drug administered. In some embodiments, a microsphere of the present disclosure provides higher loading of a therapeutic compound or salt of the present disclosure without a burst penalty. In some embodiments, a “burst penalty” refers to an increased pharmacokinetic burst (e.g., normalized to amount of drug administered) observed upon increasing loading of drug into a microsphere. That is, a burst penalty occurs when increasing drug loading in a microsphere leads to a greater pharmacokinetic burst normalized to amount of drug administered. For example, as shown in FIG. 20, the burst Cmax and burst AUC of formulation 121, when normalized to the amount of therapeutic compound in the microsphere, are disproportionately higher than those of formulation 137.

In some embodiments, a reference microsphere of the present disclosure comprises a microsphere, e.g., manufactured as described herein using the formulation described above. In some embodiments, a reference microsphere of the present disclosure refers to a numerical standard or reference measurement to which a microsphere of the present disclosure is compared.

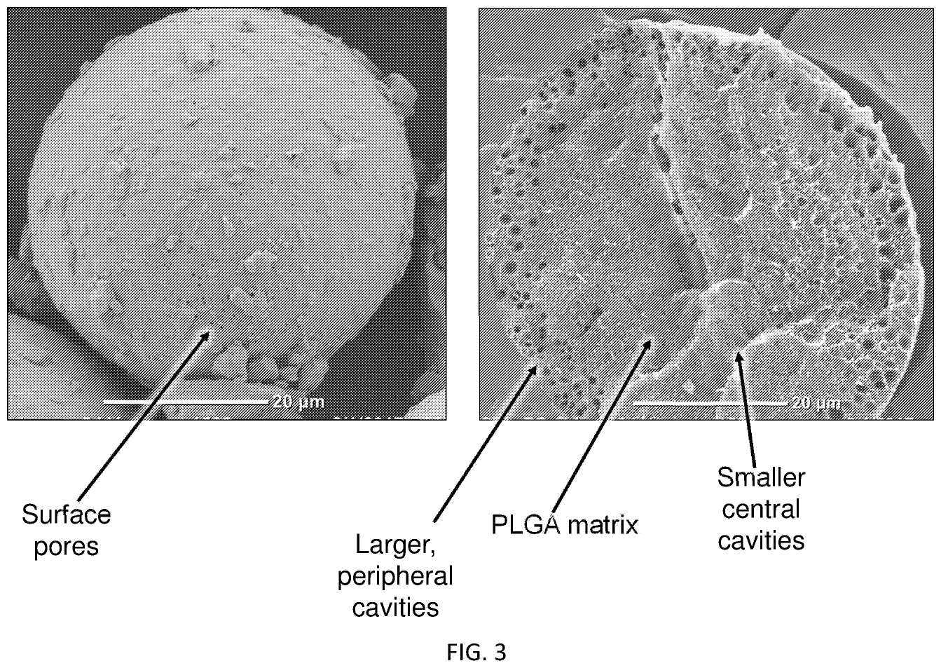

In some embodiments, burst AUC (e.g., in serum, per mg of the therapeutic compound or salt) for the microsphere of the present disclosure is equal to or less than burst AUC (e.g., in serum, per mg of the therapeutic compound or salt) for the reference microsphere. In some embodiments, burst AUC is measured 24, 48, or 72 hours after injection. In some embodiments, burst AUC is measured in serum of a mammal injected with the microsphere, such as a rabbit. Methods for measuring burst AUC are well known in the art; see, e.g., Petersen, H. et al. (2011) BMC Res. Notes 4:344 (in particular, see FIG. 3) and Example 2 for exemplary methods for measuring burst AUC.

In some embodiments, burst Cmax (e.g., in serum, per mg of the therapeutic compound or salt) for the microsphere of the present disclosure is less than burst Cmax (e.g., in serum, per mg of the therapeutic compound or salt) for the reference microsphere. In some embodiments, burst Cmax is measured within 30 minutes, 1 hour, 2 hours, or 3 hours after injection. In some embodiments, burst Cmax is measured in serum of a mammal injected with the microsphere, such as a rabbit. Methods for measuring burst Cmax are well known in the art; see, e.g., Petersen, H. et al. (2011) BMC Res. Notes 4:344 (in particular, see FIG. 3) and Example 2 for exemplary methods for measuring burst Cmax.

In some embodiments, the reference microsphere comprises the therapeutic compound or salt and the polymer(s) of the present disclosure but lacks a polyol, as compared with a microsphere of the present disclosure. For example, in some embodiments, a microsphere of the present disclosure comprises a therapeutic compound or salt of the present disclosure, a polyol, and one or more polymers of the present disclosure (e.g., where the polymer has a pI at least 1.5 units lower than the pI of the compound or salt) and induces a pharmacokinetic burst (e.g., in serum, per mg of the therapeutic compound or salt) that is equal to or less than pharmacokinetic burst (e.g., in serum, per mg of the therapeutic compound or salt) for a reference microsphere that comprises the therapeutic compound or salt and the one or more polymers but lacks the polyol. In some embodiments, a reference microsphere of the present disclosure comprises a microsphere, e.g., manufactured as described herein using the formulation described above. In some embodiments, a reference microsphere of the present disclosure refers to a numerical standard or reference measurement to which a microsphere of the present disclosure is compared.

In some embodiments, burst AUC (e.g., in serum, per mg of the therapeutic compound or salt) for the microsphere of the present disclosure is less than burst AUC (e.g., in serum, per mg of the therapeutic compound or salt) for the reference microsphere. In some embodiments, burst AUC is measured 24, 48, or 72 hours after injection. In some embodiments, burst AUC is measured in serum of a mammal injected with the microsphere, such as a rabbit. Methods for measuring burst AUC are well known in the art; see, e.g., Example 2 for an exemplary method for measuring burst AUC.

In some embodiments, burst Cmax (e.g., in serum, per mg of the therapeutic compound or salt) for the microsphere of the present disclosure is less than burst Cmax (e.g., in serum, per mg of the therapeutic compound or salt) for the reference microsphere. In some embodiments, burst Cmax is measured within 30 minutes, 1 hour, 2 hours, or 3 hours after injection. In some embodiments, burst Cmax is measured in serum of a mammal injected with the microsphere, such as a rabbit. Methods for measuring burst Cmax are well known in the art; see, e.g., Example 2 for an exemplary method for measuring burst Cmax.

In some embodiments, a microsphere of the present disclosure comprises a therapeutic compound or salt of the present disclosure, a polyol, and one or more polymers of the present disclosure (e.g., where the polymer has a pI at least 1.5 units lower than the pI of the compound or salt) and has reduced degradation of the therapeutic compound or salt as compared to a reference microsphere that comprises the therapeutic compound or salt and the one or more polymers but lacks the polyol. Methods for measuring degradation of a therapeutic compound or salt are well known in the art; see, e.g., Example 3 for an exemplary method for measuring degradation. In some embodiments, degradation is measured by measuring abundance of one or more degradation products, e.g., as compared with abundance of the non-degraded therapeutic compound or salt. In some embodiments, degradation products and/or therapeutic compound or salt are measured by mass spectrometry. In some embodiments, degradation is measured after 1 month, 2 months, 3 months, 4 months, 5 months, 6 months, or 1 year. In some embodiments, degradation at a particular temperature is measured, e.g., at 5° C., room temperature, 37° C., or 40° C. In some embodiments, a microsphere of the present disclosure comprises a therapeutic compound or salt of the present disclosure, a polyol, and one or more polymers of the present disclosure (e.g., where the polymer has a pI at least 1.5 units lower than the pI of the compound or salt) and has a longer shelf life, as compared to a reference microsphere that comprises the therapeutic compound or salt and the one or more polymers but lacks the polyol. In some embodiments, a microsphere of the present disclosure comprises a therapeutic compound or salt of the present disclosure, a polyol, and one or more polymers of the present disclosure (e.g., where the polymer has a pI at least 1.5 units lower than the pI of the compound or salt) and has less than or equal to 10%, less than or equal to 5%, or less than or equal to 1% of one or more degradation products (e.g., as normalized to amount of non-degraded compound or salt), e.g., after 1 month, 2 months, 3 months, 4 months, 5 months, 6 months, or 1 year at 5° C., room temperature, 37° C., or 40° C.

### II. Methods of Preparing Microspheres

Certain aspects of the present disclosure relate to methods of preparing a microsphere of the present disclosure, e.g., a single emulsion microsphere. Any of the therapeutic compounds/salts and/or polymer(s) (e.g., as described in section I) may find use in, or be prepared by, the methods of the present disclosure. In some embodiments, the methods comprise combining a first solvent and a therapeutic compound or pharmaceutically acceptable salt thereof to form a first mixture; combining a second solvent and one or more polymers to form a second mixture; combining the first and second mixtures to form a feed; dispersing the combined first and second mixtures of step (c) (e.g., the feed) into an aqueous continuous phase to form a droplet; and hardening the droplet formed in step (d) to form the single emulsion microsphere. In some embodiments, the methods comprise combining a first solvent and a therapeutic compound or pharmaceutically acceptable salt thereof to form a first mixture; combining a second solvent, a first polymer, and a second polymer to form a second mixture; combining the first and second mixtures to form a feed; dispersing the combined first and second mixtures of step (c) (e.g., the feed) into an aqueous continuous phase to form a droplet; and hardening the droplet formed in step (d) to form the single emulsion microsphere. In some embodiments, the polymer has a pI at least 1.5 units lower than the pI of the therapeutic compound or salt. In some embodiments, the first polymer has a lower molecular weight than the second polymer. In some embodiments, the methods comprise combining a first solvent and a therapeutic compound or pharmaceutically acceptable salt thereof to form a first mixture; combining a second solvent and one or more polymers to form a second mixture; combining the first and second mixtures to form a feed, where the feed comprises a polyol; dispersing the combined first and second mixtures of step (c) (e.g., the feed) into an aqueous continuous phase to form a droplet; and hardening the droplet formed in step (d) to form the single emulsion microsphere. In some embodiments, the polymer has a pI at least 1.5 units lower than the pI of the therapeutic compound or salt.

In some embodiments, a feed or a second mixture of the present disclosure comprises the one or more polymers at a concentration of at least about 150 mg/mL, at least about 160 mg/mL, at least about 170 mg/mL, at least about 180 mg/mL, at least about 190 mg/mL, at least about 200 mg/mL, at least about 225 mg/mL, at least about 250 mg/mL, at least about 275 mg/mL, or at least about 300 mg/mL. In some embodiments, a feed or a first mixture of the present disclosure comprises the therapeutic compound or salt at a concentration of at least about 10 mg/mL, at least about 20 mg/mL, at least about 30 mg/mL, or at least about 40 mg/mL. In some embodiments, a feed of the present disclosure comprises the one or more polymers at a concentration of at least about 200 mg/mL and the therapeutic compound or salt at a concentration of at least about 20 mg/mL.

In some embodiments, a feed of the present disclosure comprises a ratio of between 5:1 and 10:3 (first polymer:therapeutic compound or salt) by weight. In some embodiments, a feed of the present disclosure comprises a ratio of greater than any of 5:1, 6:1, 7:1, 8:1, 9:1, or 10:1 (first polymer:therapeutic compound or salt) by weight. In some embodiments, a feed of the present disclosure comprises a ratio of less than any of 10:3, 10:2, 10:1, 9:1, 8:1, 7:1, or 6:1 (first polymer:therapeutic compound or salt) by weight. That is, a feed of the present disclosure can comprise any ratio in a range of ratios having an upper limit of 10:3, 10:2, 10:1, 9:1, 8:1, 7:1, or 6:1 (first polymer:therapeutic compound or salt) and an independently selected lower limit of 5:1, 6:1, 7:1, 8:1, 9:1, or 10:1 (first polymer:therapeutic compound or salt), wherein the upper limit is greater than the lower limit. In some embodiments, a feed of the present disclosure comprises a ratio of between 10:1 and 10:3 (first polymer:therapeutic compound or salt) by weight, e.g., a ratio of 10:1, 10:2, or 10:3 (first polymer:therapeutic compound or salt) by weight.

In some embodiments, a feed of the present disclosure comprises a therapeutic compound or salt of the present disclosure at a concentration of about 10-60 mg/mL by weight. In some embodiments, a feed of the present disclosure comprises a therapeutic compound or salt of the present disclosure at a concentration of about 20-40 mg/mL by weight.

In some embodiments, a feed of the present disclosure comprises a polyol. Any of the polyols described herein may be used. In certain embodiments, the polyol comprises glycerol. In some embodiments, the polyol is present in the first mixture. In some embodiments, the polyol is solubilized in the feed.

In some embodiments, the feed comprises the polyol at a concentration of between about 0.3 mg/mL and about 1.2 mg/mL. In some embodiments, the feed comprises the polyol at a concentration of between about 0.6 mg/mL and about 0.9 mg/mL. In some embodiments, the feed comprises the polyol at a concentration of about 0.9 mg/mL.

In some embodiments, a microsphere of the present disclosure is prepared according to a formulation described in Table A.

TABLE A
Microsphere formulations

| Formulation name | #73 | #121 | #131 | #137 | #139 | #154 | #175 | #173 |
|---|---|---|---|---|---|---|---|---|
| Actual loading (%) | 4.6 | 9.7 | 3.6 | 9.0 | 5.9 | 3.8 | 9.0 | 8.4 |
| PLGA 502h (%) | 0 | 100 | 0 | 75 | 50 | 0 | 75 | 65 |
| PLGA 503h (%) | 100 | 0 | 100 | 25 | 50 | 100 | 25 | 35 |
| PLGA concentration | 200 | 200 | 200 | 200 | 200 | 200 | 200 | 200 |
| (mg/ml)* | | | | | | | | |
| Octreotide acetate | 20 | 40 | 20 | 30 | 20 | 0 | 30 | 30 |
| (mg/ml)* | | | | | | | | |
| Octreotide benzoate | 0 | 0 | 0 | 0 | 0 | 20 | 0 | 0 |
| (mg/ml)* | | | | | | | | |
| Glycerol (mg/ml)* | 0 | 0 | 0.9 | 0 | 0 | 0 | 0.9 | 0 |
| Continuous phase | 100% | 100% | 100% | 100% | 100% | 100% | 100% | 100% |
| | DCM | DCM | DCM | DCM | DCM | DCM | DCM | DCM |
| | sat. | sat., | sat. | sat., | sat., | sat. | sat., | sat., |
| | 150 mM | 0.5% | 150 mM | 0.5% | 0.5% | 150 mM | 0.5% | 0.5% |
| | NaCl, | PVA, | NaCl, | PVA, | PVA, | NaCl, | PVA, | PVA, |
| | 0.5% | 200 mM | 0.5% | 200 mM | 200 mM | 0.5% | 200 mM | 200 mM |
| | PVA, | Gly | PVA, | Gly | Gly | PVA, | Gly | Gly |
| | 50 mM | pH 9.0 | 50 mM | pH 9.0 | pH 9.0 | 50 mM | pH 9.0 | pH 9.0 |
| | Gly, | | Gly | | | Gly, | | |
| | pH 8.5 | | pH 8.5 | | | pH 8.5 | | |
| Membrane pore size | 15 | 20 | 15 | 20 | 20 | 15 | 20 | 20 |
| (μm) | | | | | | | | |

*Final concentration in dispersed phase.

In some embodiments, the methods of the present disclosure further include adjusting the pH of the aqueous continuous phase. In some embodiments, the pH of the aqueous continuous phase is adjusted to the pI of the therapeutic compound or salt minus 0.5 or greater. In some embodiments, the pH of the aqueous continuous phase is adjusted to about 8 to about 9.5, e.g., to about 8, to about 8.5, to about 9, or to about 9.5. In some embodiments, the pH of the aqueous continuous phase is adjusted to about 7.5 to about 8.5, e.g., to about 7.5, to about 8, or to about 8.5. In some embodiments, the pH of the aqueous continuous phase is adjusted with a buffer solution. A variety of buffer solutions are known in the art and may be selected by one of skill in the art; exemplary buffers include, without limitation, glycine, glycylglycine, tricine, HEPES, MOPS, sulfonate, ammonia, potassium phosphate, CHES, borate, TAPS, Tris, bicine, TAPSO, TES, and Tris buffer solutions. In some embodiments, the buffer is glycylglycine, bicine, or tricine. In some embodiments, the buffer is not Tris buffer. In some embodiments, the pH of the aqueous continuous phase is adjusted to about 8.0 in glycylglycine, bicine, or tricine buffer. In some embodiments, the pH of the aqueous continuous phase is adjusted to about 8.0 in glycylglycine buffer.

In some embodiments, the methods of the present disclosure further include (e.g., after dispersing the feed into the aqueous continuous phase to form a droplet, and prior to hardening the droplet to form a microsphere) washing the microsphere in a second aqueous continuous phase. In some embodiments, the second aqueous continuous phase is the same as (in some embodiments, has the same composition as) the first aqueous continuous phase. In some embodiments, the methods of the present disclosure further include (e.g., simultaneous with or after washing the microsphere in a second aqueous continuous phase, and prior to hardening the droplet to form a microsphere) performing size-selective filtration on the microsphere (e.g., scalping).

In some embodiments, the methods of the present disclosure further include (e.g., after initial hardening the droplet to form a micro sphere, and/or prior to optional lyophilization or spray drying) washing the microsphere in an aqueous alcohol solution, e.g., an aqueous solution comprising ethanol or isopropanol. In some embodiments, the aqueous alcohol solution comprises between about 1% and about 20% alcohol, between about 5% and about 20% alcohol, between about 5% and about 10% alcohol, between about 1% and about 15% alcohol, between about 5% and about 15% alcohol, between about 10% and about 20% alcohol, or between about 1% and about 10% alcohol (e.g., an aliphatic alcohol, such as ethanol or isopropanol). In some embodiments, the aqueous alcohol solution comprises the alcohol at about 10% or about 20% (e.g., an aliphatic alcohol, such as ethanol or isopropanol). In certain embodiments, the aqueous alcohol solution comprises about 10% ethanol or isopropanol. In some embodiments, the aqueous alcohol solution further comprises a buffer. For example, in some embodiments, the aqueous alcohol solution comprises an acetate buffer. In some embodiments, the aqueous alcohol solution is buffered to a pH of less than about 7, less than about 6, or less than about 5. In some embodiments, the aqueous alcohol solution is buffered to a pH of about 4. This optional washing step may be useful, e.g., in removing residual dichloromethane, and may result in more effective removal of residual dichloromethane than the use of an aqueous wash (e.g., a water wash lacking dichloromethane). In some embodiments, the optional washing step results in comparable or lesser amounts of residual dichloromethane in the microspheres, as compared with the residual dichloromethane present in SANDOSTATIN® LAR. In some embodiments, the optional washing step results in minimal loss of therapeutic compound or pharmaceutically acceptable salt thereof from the microspheres.

In some embodiments, hardening the droplet(s) to form single emulsion microsphere(s) of the present disclosure comprises exacervation.

In some embodiments, the methods further include (e.g., after hardening the droplet to form a microsphere) lyophilizing or spray drying the microsphere(s).

In some embodiments, the droplet is allowed to harden for at least about 60 minutes, at least about 90 minutes, or at least about 120 minutes.

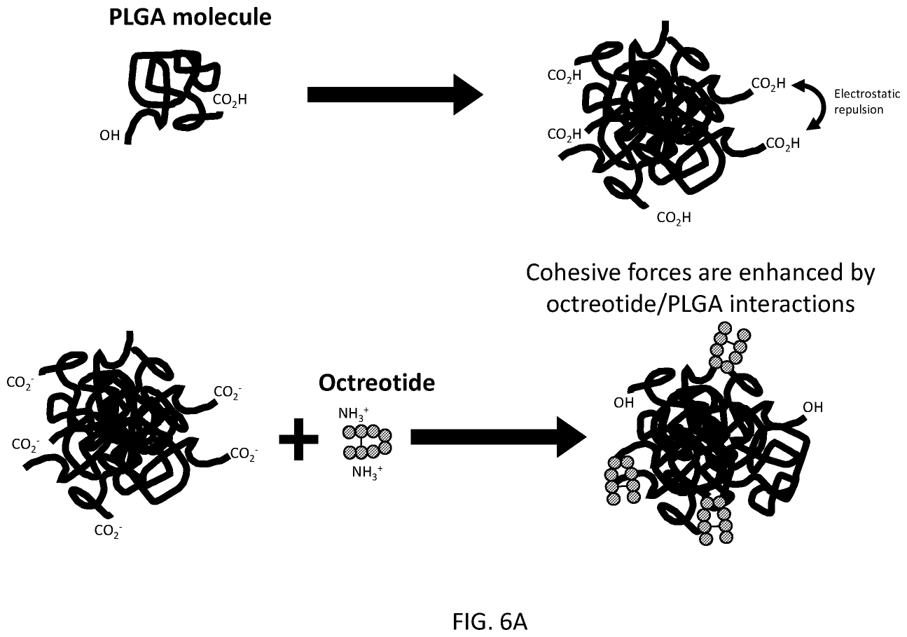

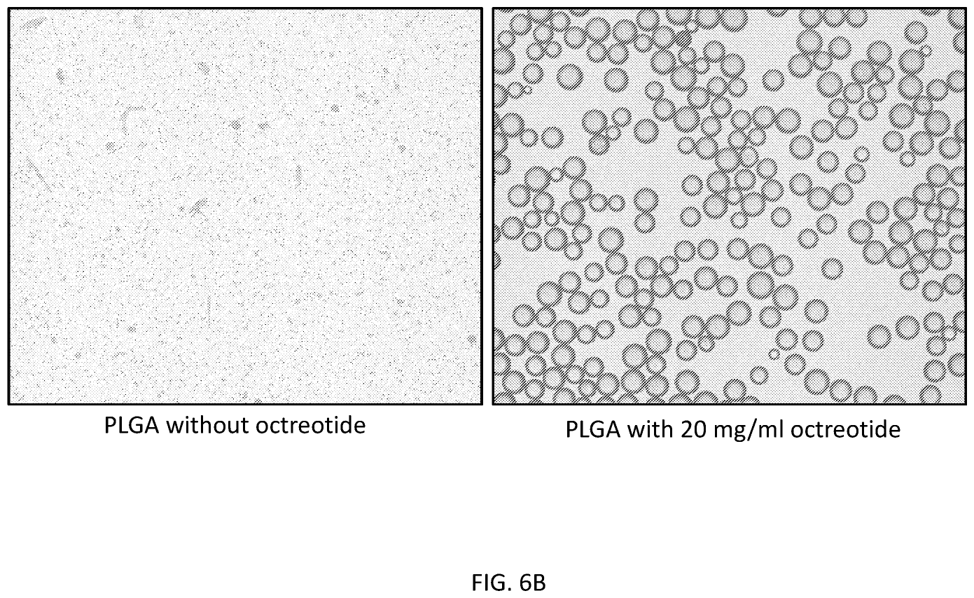

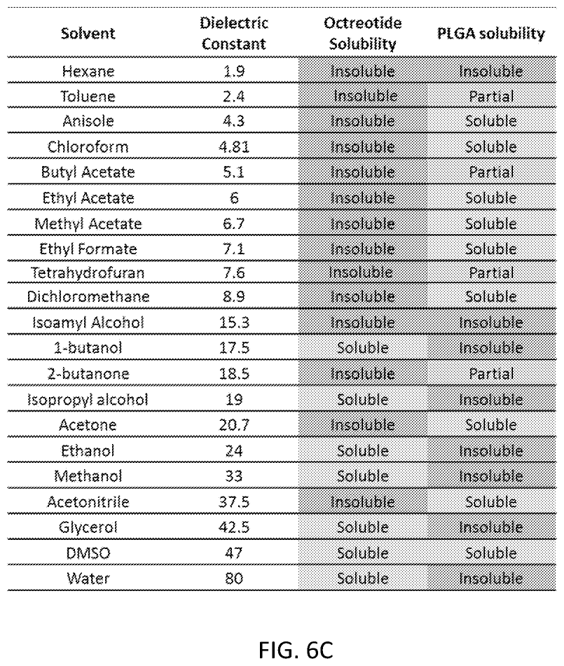

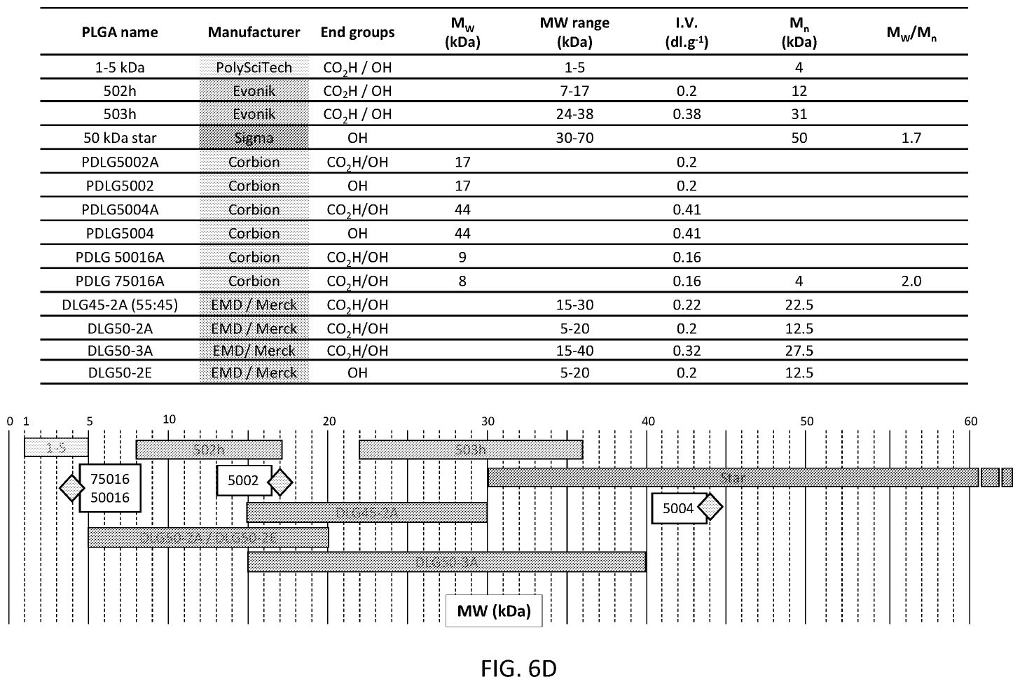

In some embodiments, a first solvent of the present disclosure comprises ethanol, propanol, or methanol. In some embodiments, a second solvent of the present disclosure comprises dichloromethane, chloroform, or ethyl acetate. Additional solvents that can be used to solubilize a therapeutic compound of the present disclosure or a polymer of the present disclosure are described in FIG. 6C.

In some embodiments, a feed is dispersed into an aqueous continuous phase using a membrane. In some embodiments, the membrane comprises a plurality of pores. In some embodiments, the membrane comprises a material treated to increase hydrophilicity of the membrane. For example, in some embodiments, the membrane is coated with a hydrophilic polymer. In some embodiments, the membrane comprises stainless steel, tantalum, tungsten, molybdenum, manganese, tin, zinc, or an alloy thereof. In some embodiments, the membrane comprises porous glass or a ceramic.

A membrane of the present disclosure may include one or more pores having a designated size (e.g., diameter), e.g., to control microsphere shape and/or size. In some embodiments, the membrane comprises pores having a size from about 5 μm to about 50 μm, from about 5 μm to about 40 μm, from about 5 μm to about 30 μm, or from about 5 μm to about 20 μm. In some embodiments, the membrane comprises pores having a size that is at least about any of the following sizes (in μm): 5, 10, 15, 20, 25, 30, 35, 40, or 45. In some embodiments, the membrane comprises pores having a size that is less than about any of the following sizes (in μm): 50, 45, 40, 35, 30, 25, 20, 15, or 10. That is, a membrane of the present disclosure can have any size in a range having an upper limit of about 50, 45, 40, 35, 30, 25, 20, 15, or 10 μm, and an independently selected lower limit of about 5, 10, 15, 20, 25, 30, 35, 40, or 45 μm, wherein the upper limit is greater than the lower limit. In some embodiments, the membrane comprises pores having a size from about 5 μm to about 50 μm, and the feed is dispersed in step (d) at a flow rate of about 130 nL/min/pore. In some embodiments, the flow rate is adjusted based on pore size, and vice versa. In some embodiments, the feed is dispersed at a flow rate of between about 0.1 nLmin⁻¹ μm⁻² (pore size) and about 1 nLmin⁻¹ μm⁻² (pore size). For example, in some embodiments, the feed is dispersed at a flow rate of about 0.41 nLmin⁻¹ μm⁻² (pore size).

In some embodiments, the flow rate of the continuous phase is about 1.5 L/min to about 3.5 L/min or about 1.7 L/min to about 3.4 L/min. In some embodiments, the flow rate of the continuous phase is about 1.7 L/min, about 2.0 L/min, about 2.5 L/min, about 3.0 L/min, or about 3.4 L/min. In certain embodiments, the flow rate of the continuous phase is about 3.4 L/min.

In some embodiments, the flow rate of the dispersed phase is about 8 mL/min to about 13 mL/min or about 9 mL/min to about 12 mL/min. In some embodiments, the flow rate of the dispersed phase is about 9 mL/min, about 10 mL/min, about 11 mL/min, or about 12 mL/min. In certain embodiments, the flow rate of the dispersed phase is about 10 mL/min. In certain embodiments, the flow rate of the dispersed phase is about 10 mL/min, and the flow rate of the continuous phase is about 3.4 L/min.

A membrane of the present disclosure may include a plurality of pores having a designated spacing (e.g., between pores), e.g., to control microsphere shape and/or size. In some embodiments, the membrane comprises pores separated by at least 40 μm, at least 50 μm, at least 60 μm, at least 70 μm, at least 80 μm, at least 90 μm, at least 100 μm, at least 125 μm, at least 150 μm, at least 175 μm, or at least 200 μm. In some embodiments, the pores are spaced in a lattice, such as a square lattice. In some embodiments, the pores can be separated by at least 40 μm spacing in the direction perpendicular to the flow, with each row of such pores offset in that perpendicular direction such that, in the direction of flow, the pores are spaced at least 200 μm apart.

In some embodiments, a feed is dispersed into an aqueous continuous phase by applying a shear force. In some embodiments, the shear force is between about 1,900 s⁻¹ and about 190,000 s⁻¹. In some embodiments, the shear force is between about 500 s⁻¹ and about 40,000 s⁻¹. In some embodiments, the shear force is at least about any of the following shear forces (in s⁻¹): 500, 1000, 1500, 1900, 2000, 2500, 5000, 7500, 10000, 20000, 30000, 40000, 50000, 60000, 70000, 80000, 90000, 100000, 110000, 120000, 130000, 140000, 150000, 160000, 170000, or 180000. In some embodiments, the shear force is less than about any of the following shear forces (in s⁻¹): 190000, 180000, 170000, 160000, 150000, 140000, 130000, 120000, 110000, 100000, 90000, 80000, 70000, 60000, 50000, 40000, 30000, 20000, 10000, 7500, 5000, 2500, 2000, 1500, or 1000. That is, the shear force can be any in a range having an upper limit of about 190000, 180000, 170000, 160000, 150000, 140000, 130000, 120000, 110000, 100000, 90000, 80000, 70000, 60000, 50000, 40000, 30000, 20000, 10000, 7500, 5000, 2500, 2000, 1500, or 1000 s⁻¹, and an independently selected lower limit of about 500, 1000, 1500, 1900, 2000, 2500, 5000, 7500, 10000, 20000, 30000, 40000, 50000, 60000, 70000, 80000, 90000, 100000, 110000, 120000, 130000, 140000, 150000, 160000, 170000, or 180000 s⁻¹, wherein the upper limit is greater than the lower limit. In some embodiments, the shear force is about 4,000 s⁻¹. In some embodiments, the shear force is obtained by laminar, plug, or turbulent flow. It is understood that any type of flow may be used provided that the shear force is sufficient to detach the droplets.

In some embodiments, an aqueous continuous phase of the present disclosure comprises a surfactant. A variety of surfactants are known in the art and can be selected by one of skill in the art. In some embodiments, the surfactant is selected from polysorbate 20 or polysorbate 80 (e.g., of the TWEEN® series), poloxamer (e.g., of the PLURONIC® series; BASF), and polyvinyl alcohol (PVA). In some embodiments, the concentration of surfactant in the aqueous continuous phase is from 0.05% to 1% (w/w). In some embodiments, the concentration of surfactant in the aqueous continuous phase is at least about any of the following concentrations (in percentage, w/w): 0.05, 0.1, 0.2, 0.3, 0.4, 0.5, 0.6, 0.7, 0.8, or 0.9. In some embodiments, the concentration of surfactant in the aqueous continuous phase is less than about any of the following concentrations (in percentage, w/w): 1, 0.9, 0.8, 0.7, 0.6, 0.5, 0.4, 0.3, 0.2, or 0.1. That is, the concentration of surfactant in the aqueous continuous phase can be any concentration in a range having an upper limit of about 1, 0.9, 0.8, 0.7, 0.6, 0.5, 0.4, 0.3, 0.2, or 0.1% (w/w), and an independently selected lower limit of about 0.05, 0.1, 0.2, 0.3, 0.4, 0.5, 0.6, 0.7, 0.8, or 0.9% (w/w), wherein the upper limit is greater than the lower limit. In some embodiments, the concentration of surfactant in the aqueous continuous phase is about 0.5% (w/w).

Advantageously, the present disclosure provides methods of generating microspheres that are substantially free of small hydrocarbons, such as C₁-C₁₆ hydrocarbons, C₁-C₁₆ alkanes or heptane, and/or silicon oil, unlike previous methods of microsphere manufacture that use heptane as an oil-in-water emulsion or wash to remove phase inducing agents such as silicon oil (see, e.g., U.S. Pat. No. 5,538,739). In some embodiments, a microsphere of the present disclosure is substantially free of small hydrocarbons (e.g., small alkanes, heptane). In some embodiments, a microsphere of the present disclosure is substantially free of silicon oil. In some embodiments, a microsphere of the present disclosure is considered “substantially free” of compounds such as small hydrocarbons (e.g., small alkanes, heptane) and/or silicon oil when it contains the compound(s) at less than 500 ppm by weight, less than 450 ppm by weight, less than 400 ppm by weight, less than 350 ppm by weight, less than 300 ppm by weight, less than 250 ppm by weight, less than 200 ppm by weight, less than 150 ppm by weight, or less than 100 ppm by weight. In some embodiments, a microsphere of the present disclosure is generated without addition of small hydrocarbons (e.g., small alkanes, heptane). In some embodiments, a microsphere of the present disclosure is generated without addition of silicon oil. In some embodiments, the methods of the present disclosure do not comprise the addition of silicon oil (e.g., as a phase inducing agent). In some embodiments, the methods of the present disclosure do not comprise the addition of heptane (e.g., as an oil-in-water emulsion or wash to remove phase inducing agents such as silicon oil).

Further provided herein are microspheres produced using a method according to any of the above embodiments.

### III. Methods of Using Microspheres

Certain aspects of the present disclosure relate to methods of treating a condition by administering to an individual (e.g., in need thereof) a therapeutically effective amount of a microsphere (see sections I and II) or pharmaceutical composition (see section IV) of the present disclosure. In some embodiments, the individual has abnormally elevated growth hormone, insulin, and/or glucagon levels. In some embodiments, the individual has a growth hormone deficiency.

In some embodiments, the condition is selected from acromegaly, carcinoid tumors, vasoactive intestinal peptide secreting tumors, diarrhea associated with acquired immune deficiency syndrome (AIDS), diarrhea associated with chemotherapy, diarrhea associated with radiation therapy, dumping syndrome, adrenal gland neuroendocrine tumors, bowel obstruction, enterocutaneous fistulae, gastrinoma, acute bleeding of gastroesophageal varices, islet cell tumors, lung neuroendocrine tumors, malignancy, meningiomas, gastrointestinal tract neuroendocrine tumors, thymus neuroendocrine tumors, pancreatic fistulas, pancreas neuroendocrine tumors, pituitary adenomas, short-bowel syndrome, small or large cell neuroendocrine tumors, thymomas and thymic carcinomas, Zollinger Ellison syndrome, acute pancreatitis, breast cancer, chylothorax, congenital lymphedema, diabetes mellitus, gastric paresis, hepatocellular carcinoma, non-variceal upper gastrointestinal bleeding, obestity, pancreaticoduodenectomy, prostate cancer, protein-losing enteropathy, small cell lung cancer, thyroid cancer, thyroid eye disease, vascular (arterio-venous) malformations of the gastrointestinal tract, polycystic kidney disease, Cushing's disease, GHRH-producing tumors, and other conditions resulting in abnormally elevated growth hormone, insulin, or glucagon levels in an individual. For additional and non-limiting examples of conditions, JAK inhibitors are in development for treating cancer, rheumatoid arthritis, dry eye disease, psoriasis, inflammatory bowel disease (IBD), transplant rejection, systemic lupus erythematosus (SLE), myelodysplastic syndrome, essential thrombocythemia, polycythemia vera, myelofibrosis, and myeloproliferative disorder; mTOR inhibitors are in development for treating cancer, transplantation (e.g., prevention of allograft rejection), restenosis, and tuberous sclerosis complex; and glucocorticoids are used for treating inflammation, rheumatoid arthritis, IBD, colitis, psoriasis, eczema, cancer, adrenal insufficiency, heart failure, allergy, asthma, skin conditions, multiple sclerosis, and during or after certain surgical procedures.

In some embodiments, the condition is alopecia. For example, provided herein are methods of treating alopecia by administering a microsphere of the present disclosure comprising a JAK inhibitor, or a pharmaceutical composition containing a microsphere of the present disclosure comprising a JAK inhibitor. In some embodiments, the administration is dermal or subdermal injection. In some embodiments, the JAK inhibitor inhibits JAK1, JAK2, and JAK3 (and optionally TYK2). In some embodiments, the JAK inhibitor inhibits JAK1. In some embodiments, the JAK inhibitor inhibits JAK3. In some embodiments, the JAK inhibitor inhibits JAK1 and JAK3.

For example, the somatostatins are indicated for use in the treatment of disorders wherein long term application of the drug is envisaged, e.g. disorders with an aetiology comprising or associated with excess GH-secretion, e.g. in the treatment of acromegaly, for use in the treatment of gastrointestinal disorders, for example, in the treatment or prophylaxis of peptic ulcers, enterocutaneous and pancreaticocutaneous fistula, irritable bowel syndrome, dumping syndrome, watery diarrhea syndrome, acute pancreatitis and gastroenteropathic endocrine tumors (e.g. vipomas, GRPomas, glucagonomas, insulinomas, gastrinomas and carcinoid tumors) as well as gastro-intestinal bleeding, breast cancer and complications associated with diabetes.

In some embodiments, the microsphere or composition is administered by injection, e.g., subcutaneous or intramuscular injection.

In some embodiments, an “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual is a human.

### IV. Pharmaceutical Compositions, Kits, and Articles of Manufacture

Certain aspects of the present disclosure relate to pharmaceutical compositions comprising a microsphere of the present disclosure (e.g., as described in section I above, or prepared by a method described in section II above). The pharmaceutical compositions may find use, e.g., in any of the methods described in section III above.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the formulation would be administered.

In some embodiments, a pharmaceutical composition of the present disclosure comprises a microsphere of the present disclosure and a pharmaceutically acceptable carrier. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, diluent, or preservative.

As is well known in the art, pharmaceutically acceptable excipients are relatively inert substances that facilitate administration of a pharmacologically effective substance and can be supplied as liquid solutions or suspensions, as emulsions, or as solid forms suitable for dissolution or suspension in liquid prior to use. For example, an excipient can give form or consistency, or act as a diluent. Suitable excipients include but are not limited to stabilizing agents, wetting and emulsifying agents, salts for varying osmolarity, encapsulating agents, pH buffering substances, and buffers. Such excipients include any pharmaceutical agent suitable for injection which may be administered without undue toxicity. Pharmaceutically acceptable excipients include, but are not limited to, sorbitol, any of the various TWEEN compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in REMINGTON'S PHARMACEUTICAL SCIENCES (Mack Pub. Co., N.J. 1991). In certain embodiments, the excipient is selected from acetate, citrate, lactate, polyols (e.g., mannitol or glycerol), carboxy-methyl cellulose and hydroxy-prolyl cellulose, and glycine.

Formulations described herein may be utilized in depot form, e.g. injectable microspheres or implants. The sustained release formulations containing octreotide may be administered for all the known indications of the octreotide or derivatives thereof, e.g. those disclosed in GB 2,199,829 A pages 89-96, as well as for acromegaly and for breast cancer. In some embodiments, the release time of the peptide from the microsphere may be from one or two weeks to about 2 months.

In some embodiments, pharmaceutically acceptable excipients may include pharmaceutically acceptable carriers. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Additional ingredients may also be used, for example preservatives, buffers, tonicity agents, antioxidants and stabilizers, nonionic wetting or clarifying agents, viscosity-increasing agents (e.g., carboxymethylcellulose or a poloxamer), and the like. The kits described herein can be packaged in single unit dosages or in multidosage forms. The contents of the kits are generally formulated as sterile and substantially isotonic solution.

In some embodiments, the kits or articles further comprise a package insert with instructions for using the microspheres or pharmaceutical compositions related thereto, e.g., in any of the methods described in section III above.

The use of the terms “a” and “an” and “the” and similar referents (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Wherever an open-ended term is used to describe a feature or element, it is specifically contemplated that a closed-ended term can be used in place of the open-ended term without departing from the spirit and scope of the disclosure. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the description and does not pose a limitation on the scope of the description unless otherwise claimed. No language in the specification should be construed as indicating any non-claimed element as essential to the practice of the compositions, methods, and kits disclosed herein.

Preferred embodiments are described herein. Variations of those preferred embodiments may become apparent to those working in the art upon reading the foregoing description. It is expected that skilled artisans will be able to employ such variations as appropriate, and the practice of the compositions, methods, and kits described herein otherwise than as specifically described herein. Accordingly, the compositions, methods, and kits described herein include all modifications and equivalents of the subject matter recited in the claims appended hereto as permitted by applicable law. Moreover, any combination of the above-described elements in all possible variations thereof is encompassed by the description unless otherwise indicated herein or otherwise clearly contradicted by context.

### LIST OF EMBODIMENTS

### Embodiment 1

A microsphere, comprising:

a therapeutic compound or pharmaceutically acceptable salt thereof having a first pI; and

a first polymer, wherein the polymer has a second pI at least 1.5 units lower than the first pI;

wherein the microsphere is a single emulsion microsphere.

### Embodiment 2

The microsphere of embodiment 1, further comprising a second polymer, wherein first polymer has a lower molecular weight than the second polymer.

### Embodiment 3

The microsphere of embodiment 2, wherein the pharmacokinetic burst in serum per mg of the therapeutic compound or salt for the microsphere is equal to or less than the pharmacokinetic burst in serum per mg of the therapeutic compound or salt for a reference microsphere.

### Embodiment 4

The microsphere of embodiment 3, wherein the reference microsphere comprises the second polymer but lacks the first polymer.

### Embodiment 5

The microsphere of embodiment 3 or embodiment 4, wherein the reference microsphere comprises the therapeutic compound or salt at a lower loading level than the microsphere.

### Embodiment 6

The microsphere of any one of embodiments 3-5, wherein the reference microsphere is a double emulsion microsphere.

### Embodiment 7

The microsphere of any one of embodiments 1-6, further comprising a polyol.

### Embodiment 8

The microsphere of embodiment 7, wherein the pharmacokinetic burst in serum per mg of the therapeutic compound or salt for the microsphere is equal to or less than the pharmacokinetic burst in serum per mg of the therapeutic compound or salt for a reference microsphere, wherein the reference microsphere comprises the therapeutic compound and the polymer but lacks the polyol.

### Embodiment 9

The microsphere of embodiment 7 or embodiment 8, wherein degradation of the therapeutic compound or salt in the microsphere is less than degradation of the therapeutic compound or salt in a reference microsphere, wherein the reference microsphere comprises the therapeutic compound and the polymer but lacks the polyol.

### Embodiment 10

The microsphere of any one of embodiments 3-9, wherein burst AUC in serum per mg of the therapeutic compound or salt for the microsphere is equal to or less than burst AUC in serum per mg of the therapeutic compound or salt for the reference microsphere.

### Embodiment 11

The microsphere of any one of embodiments 3-9, wherein burst Cmax in serum per mg of the therapeutic compound or salt for the microsphere is less than burst Cmax in serum per mg of the therapeutic compound or salt for the reference microsphere.

### Embodiment 12

The microsphere of any one of embodiments 2-11, wherein the first polymer has a molecular weight at least 10 kD lower than the second polymer.

### Embodiment 13

The microsphere of any one of embodiments 1-12, wherein the therapeutic compound or salt is greater than 5% by total weight of the microsphere.

### Embodiment 14

The microsphere of any one of embodiments 1-13, wherein the first polymer comprises at least one anionic terminus.

### Embodiment 15

The microsphere of any one of embodiments 1-14, wherein the first polymer comprises poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer.

### Embodiment 16

The microsphere of any one of embodiments 1-15, wherein the therapeutic compound or salt comprises at least one cationic moiety.

### Embodiment 17

The microsphere of any one of embodiments 1-16, wherein the microsphere is produced from a feed comprising the first polymer at a concentration of at least about 150 mg/mL and the therapeutic compound or salt at a concentration of at least about 10 mg/mL.

### Embodiment 18

The microsphere of embodiment 17, wherein the microsphere is produced from a feed comprising the first polymer at a concentration of at least about 200 mg/mL and the therapeutic compound or salt at a concentration of at least about 20 mg/mL.

### Embodiment 19

The microsphere of any one of embodiments 2-18, wherein the microsphere is produced from a feed comprising the first polymer and the second polymer at a total concentration of at least about 150 mg/mL and the therapeutic compound or salt at a concentration of at least about 10 mg/mL.

### Embodiment 20

The microsphere of embodiment 19, wherein the microsphere is produced from a feed comprising the first polymer and the second polymer at a total concentration of at least about 200 mg/mL and the therapeutic compound or salt at a concentration of at least about 20 mg/mL.

### Embodiment 21

The microsphere of any one of embodiments 1-20, wherein the molecular weight of the first polymer is less than or equal to 17 kD.

### Embodiment 22

The microsphere of any one of embodiments 2-21, wherein the second polymer comprises poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer.

### Embodiment 23

The microsphere of any one of embodiments 2-22, wherein the first and the second polymers both comprise PLGA.

### Embodiment 24

The microsphere of any one of embodiments 2-23, wherein the microsphere comprises the first polymer and the second polymer at a ratio of between about 20:80 and about 80:20 (first polymer:second polymer).

### Embodiment 25

The microsphere of embodiment 24, wherein the microsphere comprises the first polymer and the second polymer at a ratio of about 75:25 (first polymer:second polymer).

### Embodiment 26

The microsphere of embodiment 24, wherein the microsphere comprises the first polymer and the second polymer at a ratio of about 65:35 (first polymer:second polymer).

### Embodiment 27

The microsphere of any one of embodiments 7-26, wherein the polyol is glycerol.

### Embodiment 28

The microsphere of any one of embodiments 1-27, wherein the therapeutic compound comprises a therapeutic peptide.

### Embodiment 29

The microsphere of embodiment 28, wherein the therapeutic peptide comprises at least two amino-containing amino acid side chains.

### Embodiment 30

The microsphere of embodiment 28 or embodiment 29, wherein the therapeutic peptide has a length from 6 to 40 amino acids.

### Embodiment 31

The microsphere of any one of embodiments 28-30, wherein the therapeutic peptide has a length of 8 amino acids.

### Embodiment 32

The microsphere of any one of embodiments 28-31, wherein the therapeutic peptide is cyclic.

### Embodiment 33

The microsphere of any one of embodiments 28-32, wherein the therapeutic peptide is a somatostatin analog or a pharmaceutically acceptable salt thereof.

### Embodiment 34

The microsphere of any one of embodiments 28-33, wherein the therapeutic peptide is selected from the group consisting of somatostatin (SST-28), SST-14, lanreotide, octreotide, vapreotide, pasireotide, and pharmaceutically acceptable salts of any of the foregoing.

### Embodiment 35

The microsphere of any one of embodiments 1-27, wherein the therapeutic compound comprises a glucocorticoid, JAK inhibitor, or mTOR inhibitor.

### Embodiment 36

The microsphere of embodiment 35, wherein the therapeutic compound comprises a JAK inhibitor that inhibits JAK1, JAK3, JAK1 and JAK3, or JAK1, JAK2, and JAK3.

### Embodiment 37

The microsphere of embodiment 35, wherein the therapeutic compound comprises a JAK inhibitor selected from the group consisting of ruxolitinib, tofacitinib, oclacitinib, baricitinib, filgotinib, gandotinib, lestaurtinib, momelotinib, pacritinib, PF-04965842, upadacitinib, peficitinib, fedratinib, cucurbitacin I, decernotinib, INCB018424, AC430, BMS-0911543, GSK2586184, VX-509, R348, AZD1480, CHZ868, PF-956980, AG490, WP-1034, JAK3 inhibitor IV, atiprimod, FM-381, SAR20347, AZD4205, ARN4079, NIBR-3049, PRN371, PF-06651600, JAK3i, JAK3 inhibitor 31, PF-06700841, NC1153, EP009, Gingerenone A, JANEX-1, cercosporamide, JAK3-IN-2, PF-956980, Tyk2-IN-30, Tyk2-IN-2, JAK3-IN1, WHI-P97, TG-101209, AZ960, NVP-BSK805, NSC 42834, FLLL32, SD 1029, WIH-P154, WHI-P154, TCS21311, JAK3-IN-1, JAK3-IN-6, JAK3-IN-7, XL019, MS-1020, AZD1418, WP1066, CEP33779, ZM 449829, SHR0302, JAK1-IN-31, WYE-151650, EXEL-8232, solcitinib, itacitinib, cerdulatinib, PF-06263276, delgotinib, AS2553627, JAK-IN-35, ASN-002, AT9283, diosgenin, JAK inhibitor 1, JAK-IN-1, LFM-A13, NS-018, RGB-286638, SB1317, curcumol, Go6976, JAK2 inhibitor G5-7, myricetin, and pyridine 6.

### Embodiment 38

The microsphere of any one of embodiments 1-37, wherein the microsphere is substantially free of small hydrocarbons and/or silicon oil.

### Embodiment 39

A method of preparing a single emulsion microsphere, comprising the steps of:

a) combining a first solvent and a therapeutic compound or pharmaceutically acceptable salt thereof to form a first mixture, wherein the compound or salt has a first pI;

b) combining a second solvent and a first polymer to form a second mixture, wherein the polymer has a second pI at least 1.5 units lower than the first pI;

c) combining the first and second mixtures to form a feed;

d) dispersing the combined first and second mixtures of step (c) into an aqueous continuous phase to form a droplet; and

e) hardening the droplet formed in step (d) to form the single emulsion microsphere.

### Embodiment 40

The method of embodiment 39, wherein step b) further comprises combining a second polymer with the second solvent and the first polymer to form the mixture.

### Embodiment 41

The method of embodiment 40, wherein the first polymer has a lower molecular weight than the second polymer.

### Embodiment 42

The method of embodiment 41, wherein the pharmacokinetic burst in serum per mg of the therapeutic compound or salt for the microsphere is equal to or less than pharmacokinetic burst in serum per mg of the therapeutic compound or salt for a reference microsphere.

### Embodiment 43

The method of embodiment 42, wherein the reference microsphere comprises the second polymer but lacks the first polymer.

### Embodiment 44

The method of embodiment 42 or embodiment 43, wherein the reference microsphere comprises the therapeutic compound or salt at a lower loading level than the microsphere formed in step (e).

### Embodiment 45

The method of embodiment 44, wherein the microsphere formed in step (e) does not induce a burst penalty as compared with the reference microsphere.

### Embodiment 46

The method of embodiment 44 or embodiment 45, wherein the reference microsphere is a double emulsion microsphere.

### Embodiment 47

The method of any one of embodiments 39-46, wherein the feed further comprises a polyol.

### Embodiment 48

The method of embodiment 47, wherein the polyol is solubilized in the feed.

### Embodiment 49

The method of embodiment 47 or embodiment 48, wherein the pharmacokinetic burst in serum per mg of the therapeutic compound or salt for the microsphere is less than the pharmacokinetic burst in serum per mg of the therapeutic compound or salt for a reference microsphere, wherein the reference microsphere comprises the therapeutic compound and the polymer but lacks the polyol.

### Embodiment 50

The method of any one of embodiments 47-49, wherein degradation of the therapeutic compound or salt in the microsphere is less than degradation of the therapeutic compound or salt in a reference microsphere, wherein the reference microsphere comprises the therapeutic compound and the polymer but lacks the polyol.

### Embodiment 51

The method of any one of embodiments 42-50, wherein burst AUC in serum per mg of the therapeutic compound or salt for the microsphere is equal to or less than burst AUC in serum per mg of the therapeutic compound or salt for the reference microsphere.

### Embodiment 52

The method of any one of embodiments 42-50, wherein burst Cmax in serum per mg of the therapeutic compound or salt for the microsphere is less than burst Cmax in serum per mg of the therapeutic compound or salt for the reference microsphere.

### Embodiment 53

The method of any one of embodiments 40-52, wherein the first polymer has a molecular weight at least 10 kD lower than the second polymer.

### Embodiment 54

The method of any one of embodiments 39-53, wherein the microsphere comprises greater than 5% by total weight of the therapeutic compound or salt.

### Embodiment 55

The method of any one of embodiments 39-54, wherein the first polymer comprises at least one anionic terminus.

### Embodiment 56

The method of any one of embodiments 39-55, wherein the first polymer comprises poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer.

### Embodiment 57

The method of any one of embodiments 39-56, wherein the therapeutic compound or salt comprises at least one cationic moiety.

### Embodiment 58

The method of any one of embodiments 39-57, wherein the feed comprises the first polymer at a concentration of at least about 150 mg/mL and the therapeutic compound or salt at a concentration of at least about 10 mg/mL.

### Embodiment 59

The method of embodiment 58, wherein the feed comprises the first polymer at a concentration of at least about 200 mg/mL and the therapeutic compound or salt at a concentration of at least about 20 mg/mL.

### Embodiment 60

The method of any one of embodiments 39-57, wherein the feed comprises the first polymer and the second polymer at a total concentration of at least about 150 mg/mL and the therapeutic compound or salt at a concentration of at least about 10 mg/mL.

### Embodiment 61

The method of embodiment 60, wherein the feed comprises the first polymer and the second polymer at a total concentration of at least about 200 mg/mL and the therapeutic compound or salt at a concentration of at least about 20 mg/mL.

### Embodiment 62

The method of any one of embodiments 39-61, wherein the molecular weight of the first polymer is less than or equal to 17 kD.

### Embodiment 63

The method of any one of embodiments 40-62, wherein the second polymer comprises poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer.

### Embodiment 64

The method of any one of embodiments 40-63, wherein the first and the second polymers both comprise PLGA, and wherein the molecular weight of the first polymer is at least 10 kD lower than the molecular weight of the second polymer.

### Embodiment 65

The method of any one of embodiments 40-64, wherein the microsphere comprises the first polymer and the second polymer at a ratio of between about 20:80 and about 80:20 (first polymer:second polymer).

### Embodiment 66

The method of embodiment 65, wherein the microsphere comprises the first polymer and the second polymer at a ratio of about 75:25 (first polymer:second polymer).

### Embodiment 67

The method of embodiment 65, wherein the microsphere comprises the first polymer and the second polymer at a ratio of about 65:35 (first polymer:second polymer).

### Embodiment 68

The method of any one of embodiments 47-67, wherein the feed comprises the polyol at a concentration of about between about 0.3 mg/mL and about 1.2 mg/mL.

### Embodiment 69

The method of any one of embodiments 47-68, wherein the feed comprises the polyol at a concentration of about 0.9 mg/mL.

### Embodiment 70

The method of any one of embodiments 47-69, wherein the polyol is glycerol.

### Embodiment 71

The method of any one of embodiments 39-70, wherein the therapeutic compound comprises a therapeutic peptide.

### Embodiment 72

The method of any one of embodiments 39-71, wherein the therapeutic peptide comprises at least two amino-containing amino acid side chains.

### Embodiment 73

The method of any one of embodiments 39-72, wherein the therapeutic peptide has a length from 6 to 40 amino acids.

### Embodiment 74

The method of any one of embodiments 39-73, wherein the therapeutic peptide has a length of 8 amino acids.

### Embodiment 75

The method of any one of embodiments 39-74, wherein the therapeutic peptide is cyclic.

### Embodiment 76

The method of any one of embodiments 39-75, wherein the therapeutic peptide is a somatostatin analog or a pharmaceutically acceptable salt thereof.

### Embodiment 77

The method of any one of embodiments 39-77, wherein the therapeutic peptide is selected from the group consisting of somatostatin (SST-28), SST-14, lanreotide, octreotide, vapreotide, pasireotide, and pharmaceutically acceptable salts of any of the foregoing.

### Embodiment 78

The method of any one of embodiments 39-70, wherein the therapeutic compound comprises a glucocorticoid, JAK inhibitor, or mTOR inhibitor.

### Embodiment 79

The method of embodiment 78, wherein the therapeutic compound comprises a JAK inhibitor that inhibits JAK1, JAK3, JAK1 and JAK3, or JAK1, JAK2, and JAK3.

### Embodiment 80

The method of embodiment 78, wherein the therapeutic compound comprises a JAK inhibitor selected from the group consisting of ruxolitinib, tofacitinib, oclacitinib, baricitinib, filgotinib, gandotinib, lestaurtinib, momelotinib, pacritinib, PF-04965842, upadacitinib, peficitinib, fedratinib, cucurbitacin I, decernotinib, INCB018424, AC430, BMS-0911543, GSK2586184, VX-509, R348, AZD1480, CHZ868, PF-956980, AG490, WP-1034, JAK3 inhibitor IV, atiprimod, FM-381, SAR20347, AZD4205, ARN4079, NIBR-3049, PRN371, PF-06651600, JAK3i, JAK3 inhibitor 31, PF-06700841, NC1153, EP009, Gingerenone A, JANEX-1, cercosporamide, JAK3-IN-2, PF-956980, Tyk2-IN-30, Tyk2-IN-2, JAK3-IN1, WHI-P97, TG-101209, AZ960, NVP-BSK805, NSC 42834, FLLL32, SD 1029, WIH-P154, WHI-P154, TCS21311, JAK3-IN-1, JAK3-IN-6, JAK3-IN-7, XL019, MS-1020, AZD1418, WP1066, CEP33779, ZM 449829, SHR0302, JAK1-IN-31, WYE-151650, EXEL-8232, solcitinib, itacitinib, cerdulatinib, PF-06263276, delgotinib, AS2553627, JAK-IN-35, ASN-002, AT9283, diosgenin, JAK inhibitor 1, JAK-IN-1, LFM-A13, NS-018, RGB-286638, SB1317, curcumol, Go6976, JAK2 inhibitor G5-7, myricetin, and pyridine 6.

### Embodiment 81

The method of any one of embodiments 39-80, further comprising adjusting the pH of the aqueous continuous phase into which the feed is dispersed in step (d).

### Embodiment 82

The method of embodiment 81, wherein the pH of the aqueous continuous phase is adjusted with a buffer solution selected from the group consisting of glycine, glycyl-glycine, tricine, HEPES, MOPS, sulfonate, ammonia, potassium phosphate, CHES, borate, TAPS, Tris, bicine, TAPSO, TES, and Tris buffer solutions.

### Embodiment 83

The method of embodiment 81, wherein the pH of the aqueous continuous phase is adjusted with a buffer solution selected from the group consisting of glycylglycine, bicine, and tricine.

### Embodiment 84

The method of any one of embodiments 81-83, wherein the pH of the aqueous continuous phase is adjusted to the first pI minus 0.5 or greater.

### Embodiment 85

The method of embodiment 84, wherein the pH of the aqueous continuous phase is adjusted to about 8 to about 9.5.

### Embodiment 86

The method of embodiment 85, wherein the pH of the aqueous continuous phase is adjusted to about 9.

### Embodiment 87

The method of embodiment 84, wherein the pH of the aqueous continuous phase is adjusted to about 7.5 to about 8.5.

### Embodiment 88

The method of embodiment 87, wherein the pH of the aqueous continuous phase is adjusted to about 8.

### Embodiment 89

The method of any one of embodiments 39-88, wherein the droplet is allowed to harden in (e) for at least about 120 minutes.

### Embodiment 90

The method of any one of embodiments 39-89, wherein a plurality of microspheres are produced, and wherein at least 90% of the microspheres of the plurality are 22-36 μm in diameter.

### Embodiment 91

The method of any one of embodiments 39-89, wherein a plurality of microspheres are produced, and wherein at least 60% of the microspheres of the plurality are 26-34 μm in diameter.

### Embodiment 92

The method of any one of embodiments 39-91, further comprising, after step (d) and prior to step (e), washing the microsphere in a second aqueous continuous phase.

### Embodiment 93

The method of embodiment 92, further comprising, after washing the microsphere in a second aqueous continuous phase, performing size-selective filtration on the microsphere.

### Embodiment 94

The method of embodiment 92 or embodiment 93, wherein the second aqueous continuous phase has the same composition as the first aqueous continuous phase.

### Embodiment 95

The method of any one of embodiments 39-94, further comprising, after step (e), washing the microsphere in an aqueous alcohol solution.

### Embodiment 96

The method of embodiment 95, wherein the aqueous alcohol solution comprises an aliphatic alcohol at a concentration of between about 1% and about 20%.

### Embodiment 97

The method of embodiment 96, wherein the aqueous alcohol solution comprises ethanol at a concentration of about 10%.

### Embodiment 98

The method of any one of embodiments 95-97, wherein the aqueous alcohol solution further comprises a buffer.

### Embodiment 99

The method of embodiment 98, wherein the buffer is an acetate buffer.

### Embodiment 100

The method of embodiment 98 or embodiment 99, wherein the aqueous alcohol solution is buffered to a pH of about 4.

### Embodiment 101

The method of any one of embodiments 39-100, further comprising, after step (e), lyophilizing the microsphere.

### Embodiment 102

The method of any one of embodiments 39-100, further comprising, after step (e), spray drying the microsphere.

### Embodiment 103

The method of any one of embodiments 39-102, wherein the feed of step (c) comprises a ratio of between 10:1 and 10:3 (first polymer:therapeutic compound or salt) by weight.

### Embodiment 104

The method of any one of embodiments 39-103, wherein the feed of step (c) comprises the therapeutic compound or salt at a concentration of between about 10 mg/mL and about 60 mg/mL by weight.

### Embodiment 105

The method of any one of embodiments 39-104, wherein hardening the droplet in step (e) comprises exacervation.

### Embodiment 106

The method of any one of embodiments 39-105, wherein the first solvent comprises ethanol, propanol, or methanol.

### Embodiment 107

The method of any one of embodiments 39-106, wherein the second solvent comprises dichloromethane, chloroform, or ethyl acetate.

### Embodiment 108

The method of any one of embodiments 39-107, wherein the method does not comprise the addition of a small hydrocarbon and/or silicon oil.

### Embodiment 109

The method of any one of embodiments 39-108, wherein dispersing the feed in step (d) comprises use of a membrane.

### Embodiment 110

The method of embodiment 109, wherein the membrane comprises a material treated to increase hydrophilicity of the membrane.

### Embodiment 111

The method of embodiment 110, wherein the membrane is coated with a hydrophilic polymer.

### Embodiment 112

The method of any one of embodiments 109-111, wherein the membrane comprises stainless steel, tantalum, tungsten, molybdenum, manganese, tin, zinc, or an alloy thereof.

### Embodiment 113

The method of any one of embodiments 109-111, wherein the membrane comprises porous glass or a ceramic.

### Embodiment 114

The method of any one of embodiments 109-113, wherein the membrane comprises pores having a size from about 5 μm to about 50 μm.

### Embodiment 115

The method of embodiment 114, wherein the membrane comprises pores having a size from about 5 μm to about 20 μm.

### Embodiment 116

The method of any one of embodiments 109-115, wherein the membrane comprises pores having a size from about 5 μm to about 50 μm, and wherein the feed is dispersed in step (d) at a flow rate of about 130 nL/min/pore.

### Embodiment 117

The method of any one of embodiments 109-115, wherein the feed is dispersed in step (d) at a flow rate of between about 0.1 nLmin⁻¹ μm⁻² (pore size) and about 1 nLmin⁻¹ μm⁻² (pore size).

### Embodiment 118

The method of any one of embodiments 39-117, wherein the feed is dispersed in step (d) by applying shear force.

### Embodiment 119

The method of embodiment 118, wherein the shear force is between about 500 s⁻¹ and about 40,000 s⁻¹.

### Embodiment 120

The method of any one of embodiments 39-119, wherein the aqueous continuous phase further comprises a surfactant.

### Embodiment 121

The method of embodiment 120, wherein the surfactant is selected from the group consisting of polysorbate 20, polysorbate 80, poloxamer, and polyvinyl alcohol (PVA).

### Embodiment 122

The method of embodiment 120 or embodiment 121, wherein the concentration of the surfactant in the aqueous continuous phase is from 0.05% to 1% (w/w).

### Embodiment 123

The method of embodiment 122, wherein the concentration of the surfactant in the aqueous continuous phase is about 0.5% (w/w).

### Embodiment 124

A microsphere produced by the method of any one of embodiments 39-123.

### Embodiment 125

A pharmaceutical composition comprising the microsphere of any one of embodiments 1-38 and 124.

### Embodiment 126

A method of treating a condition, comprising:

administering to the individual a therapeutically effective amount of the microsphere of any one of embodiments 1-38 and 124 or the composition of embodiment 125,

wherein the condition is selected from the group consisting of acromegaly, carcinoid tumors, vasoactive intestinal peptide secreting tumors, diarrhea associated with acquired immune deficiency syndrome (AIDS), diarrhea associated with chemotherapy, diarrhea associated with radiation therapy, dumping syndrome, adrenal gland neuroendocrine tumors, bowel obstruction, enterocutaneous fistulae, gastrinoma, acute bleeding of gastroesophageal varices, islet cell tumors, lung neuroendocrine tumors, malignancy, meningiomas, gastrointestinal tract neuroendocrine tumors, thymus neuroendocrine tumors, pancreatic fistulas, pancreas neuroendocrine tumors, pituitary adenomas, short-bowel syndrome, small or large cell neuroendocrine tumors, thymomas and thymic carcinomas, Zollinger Ellison syndrome, acute pancreatitis, breast cancer, chylothorax, congenital lymphedema, diabetes mellitus, gastric paresis, hepatocellular carcinoma, non-variceal upper gastrointestinal bleeding, obestity, pancreaticoduodenectomy, prostate cancer, protein-losing enteropathy, small cell lung cancer, thyroid cancer, thyroid eye disease, vascular (arterio-venous) malformations of the gastrointestinal tract, polycystic kidney disease, Cushing's disease, GHRH-producing tumors, and other conditions resulting in abnormally elevated growth hormone, insulin, or glucagon levels in an individual in need thereof.

### Embodiment 127

A method of treating growth hormone deficiency, comprising:

administering to an individual in need thereof a therapeutically effective amount of the microsphere of any one of embodiments 1-38 and 124 or the composition of embodiment 125.

### Embodiment 128

The method of embodiment 126 or embodiment 127, wherein the microsphere or composition is administered to the individual by injection.

### Embodiment 129

The method of embodiment 128, wherein the injection is a subcutaneous or intramuscular injection.

### Embodiment 130

A method of treating alopecia, comprising:

administering to an individual in need thereof a therapeutically effective amount of the microsphere of any one of embodiments 1-38 and 124 or the composition of embodiment 125, wherein the therapeutic compound or pharmaceutically acceptable salt thereof is a JAK inhibitor.

### Embodiment 131

The method of embodiment 130, wherein the therapeutic compound comprises a JAK inhibitor that inhibits JAK1, JAK3, JAK1 and JAK3, or JAK1, JAK2, and JAK3.

### Embodiment 132

The method of embodiment 130, wherein the therapeutic compound comprises a JAK inhibitor selected from the group consisting of ruxolitinib, tofacitinib, oclacitinib, baricitinib, filgotinib, gandotinib, lestaurtinib, momelotinib, pacritinib, PF-04965842, upadacitinib, peficitinib, fedratinib, cucurbitacin I, decernotinib, INCB018424, AC430, BMS-0911543, GSK2586184, VX-509, R348, AZD1480, CHZ868, PF-956980, AG490, WP-1034, JAK3 inhibitor IV, atiprimod, FM-381, SAR20347, AZD4205, ARN4079, NIBR-3049, PRN371, PF-06651600, JAK3i, JAK3 inhibitor 31, PF-06700841, NC1153, EP009, Gingerenone A, JANEX-1, cercosporamide, JAK3-IN-2, PF-956980, Tyk2-IN-30, Tyk2-IN-2, JAK3-IN1, WHI-P97, TG-101209, AZ960, NVP-BSK805, NSC 42834, FLLL32, SD 1029, WIH-P154, WHI-P154, TCS21311, JAK3-IN-1, JAK3-IN-6, JAK3-IN-7, XL019, MS-1020, AZD1418, WP1066, CEP33779, ZM 449829, SHR0302, JAK1-IN-31, WYE-151650, EXEL-8232, solcitinib, itacitinib, cerdulatinib, PF-06263276, delgotinib, AS2553627, JAK-IN-35, ASN-002, AT9283, diosgenin, JAK inhibitor 1, JAK-IN-1, LFM-A13, NS-018, RGB-286638, SB1317, curcumol, Go6976, JAK2 inhibitor G5-7, myricetin, and pyridine 6.

### Embodiment 133

The method of any one of embodiments 130-132, wherein the microsphere or composition is administered to the individual by dermal or subdermal injection.

### Embodiment 134

The method of any one of embodiments 126-133, wherein the individual is a human.

### EXAMPLES

The present disclosure will be more fully understood by reference to the following examples. The examples should not, however, be construed as limiting the scope of the present disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Improved Formulation of Therapeutic Compounds into Microspheres

Background

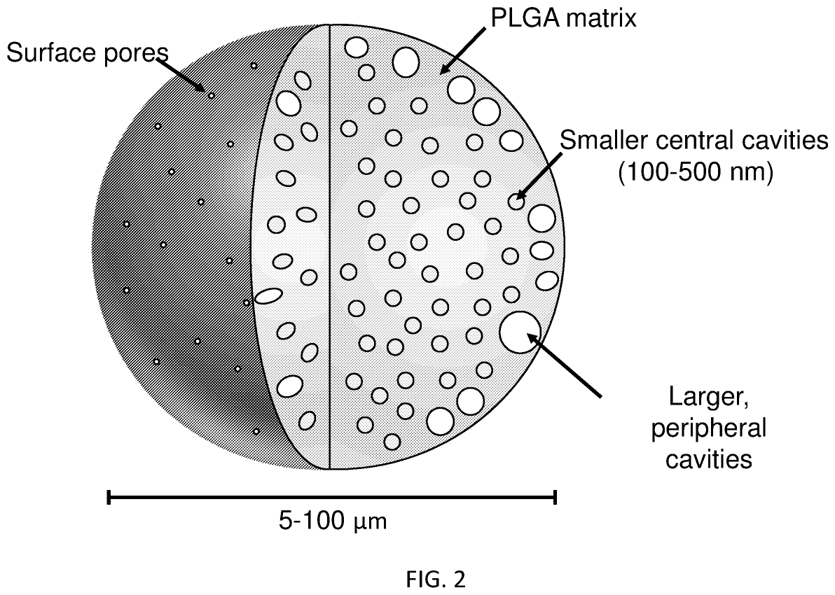

Formation of microspheres containing a therapeutic compound and a polymer is affected, inter alia, by differing solvation needs of these components. For example, the PLGA species shown in FIG. 1A is a polymer with free carboxyl and hydroxyl groups at the ends, whereas the therapeutic compound octreotide has hydrophilic groups as well as bulky hydrophobic moieties. Generation of double emulsion, water/oil/water microspheres with octreotide and PLGA (e.g., as in the octreotide acetate formulation SANDOSTATIN® LAR depot) leads to a microsphere structure with a PLGA matrix surrounding aqueous inclusions that are eventually dried to leave smaller, 100-500 nm central cavities (FIG. 2). These microspheres range from 5-100 μm in diameter.

Analysis of these microspheres using SEM revealed a surface dotted with pores and an interior characterized by a PLGA matrix, large peripheral cavities, and smaller central cavities (FIG. 3). This structure is derived from the method by which SANDOSTATIN® LAR depot is manufactured. First, a solution of octreotide in water is mixed with a solution of PLGA in organic solvent by high shear mixing (e.g., 20,000 rpm), generating an unstable water/oil emulsion. This unstable emulsion is further emulsified in an aqueous phase containing a surfactant, leading to a water/oil/water emulsion that is then hardened and lyophilized into microspheres.

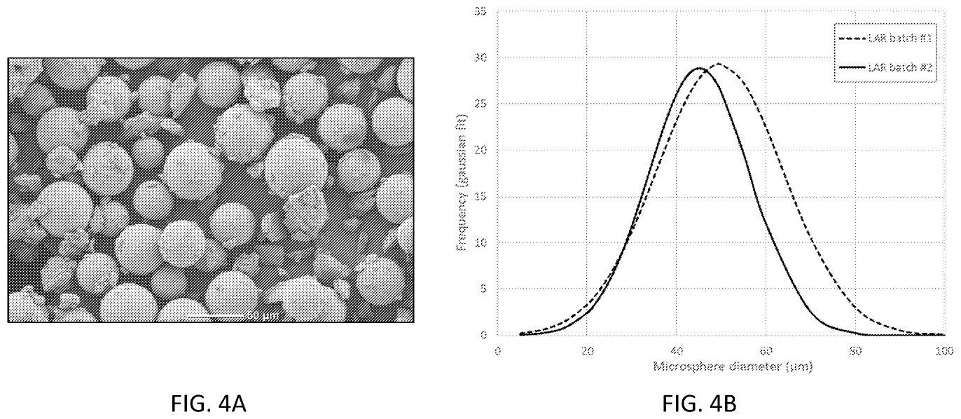

This high shear, high energy process is difficult to control or reproduce, leading to a heterogeneous preparation of double emulsion microspheres. Analysis of SANDOSTATIN® LAR microspheres by SEM revealed a population with irregular surfaces and a wide distribution of sizes (FIG. 4A). Without wishing to be bound to theory, it is thought that this porosity may increase the amount of burst drug release on injection and that surface roughness may reduce the ability of microsphere formulations to flow smoothly as a dry powder. Multiple samples were also found to contain a broad distribution of microsphere size ranging in diameter from 20 μm or less to 80-100 μm (FIG. 4B). This broad size distribution leads to problems with drug delivery such as syringeability. A broad distribution with many larger microspheres may lead to bridging of the microspheres in an arch-like configuration that blocks the needle. These and other properties have made depot formulations such as SANDOSTATIN® LAR (octreotide) and SIGNIFOR® LAR (pasireotide) notoriously difficult to inject, requiring the use of large gauge needles (18 G-20 G) and alternating injection sites.

Without wishing to be bound to theory, it is thought that a more uniform distribution with smaller microspheres can improve flow through the needle and reduce the apparent viscosity of the drug formulation. This would allow for easier administration and smaller needles. Single emulsion particles would also lead to easier manufacturing. The Examples that follow describe methods of making microspheres with improved physical and pharmacokinetic properties.

Methods

### Formulations

Octreotide:PLGA microspheres were generated according to the formulations shown in Table A. Concentrations refer to the mixture prior to extrusion.

TABLE A
Microsphere formulations

| Formulation name | #73 | #121 | #131 | #137 | #139 | #154 | #175 | #173 |
|---|---|---|---|---|---|---|---|---|
| Actual loading (%) | 4.6 | 9.7 | 3.6 | 9.0 | 5.9 | 3.8 | 9.0 | 8.4 |
| PLGA 502h (%) | 0 | 100 | 0 | 75 | 50 | 0 | 75 | 65 |
| PLGA 503h (%) | 100 | 0 | 100 | 25 | 50 | 100 | 25 | 35 |
| PLGA concentration | 200 | 200 | 200 | 200 | 200 | 200 | 200 | 200 |
| (mg/ml)* | | | | | | | | |
| Octreotide acetate | 20 | 40 | 20 | 30 | 20 | 0 | 30 | 30 |
| (mg/ml)* | | | | | | | | |
| Octreotide benzoate | 0 | 0 | 0 | 0 | 0 | 20 | 0 | 0 |
| (mg/ml)* | | | | | | | | |
| Glycerol (mg/ml)* | 0 | 0 | 0.9 | 0 | 0 | 0 | 0.9 | 0 |
| Continuous phase | 100% | 100% | 100% | 100% | 100% | 100% | 100 % | 100 % |
| | DCM | DCM | DCM | DCM | DCM | DCM | DCM | DCM |
| | sat. | sat., | sat. | sat., | sat., | sat. | sat., | sat., |
| | 150 mM | 0.5% | 150 mM | 0.5% | 0.5% | 150 mM | 0.5% | 0.5% |
| | NaCl, | PVA, | NaCl, | PVA, | PVA, | NaCl, | PVA, | PVA, |
| | 0.5% | 200 mM | 0.5% | 200 mM | 200 mM | 0.5% | 200 mM | 200 mM |
| | PVA, | Gly | PVA, | Gly | Gly | PVA, | Gly | Gly |
| | 50 mM | pH 9.0 | 50 mM | pH 9.0 | pH 9.0 | 50 mM | pH 9.0 | pH 9.0 |
| | Gly, | | Gly | | | Gly, | | |
| | pH 8.5 | | pH 8.5 | | | pH 8.5 | | |
| Membrane pore size | 15 | 20 | 15 | 20 | 20 | 15 | 20 | 20 |
| (μm) | | | | | | | | |

*Final concentration in dispersed phase.

### Microsphere Production

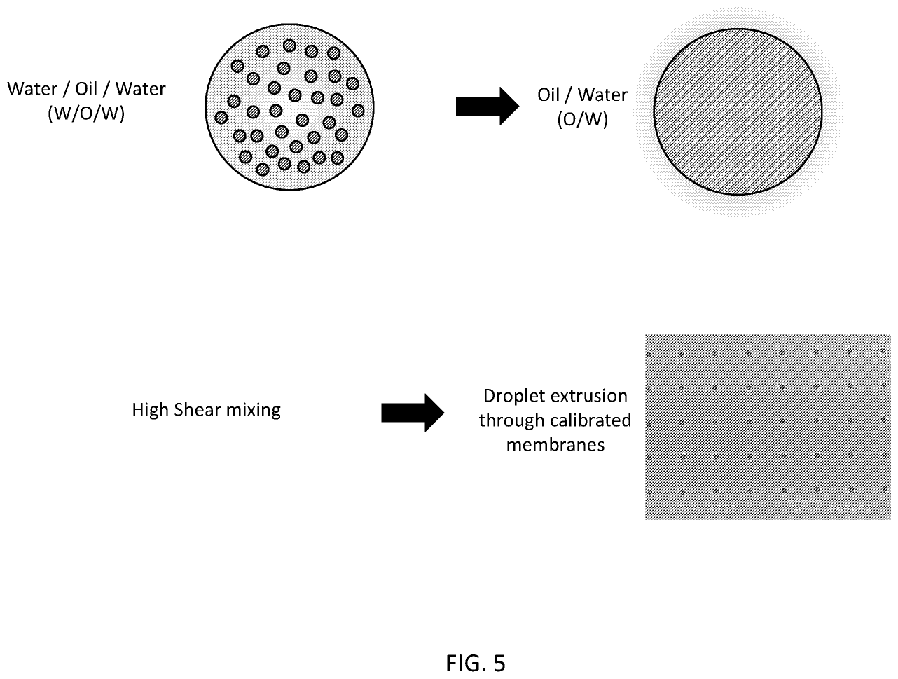

Briefly, microspheres were generated as follows. Octreotide was dissolved in ethanol, and PLGA was dissolved in methylene chloride. Both mixtures were then combined and extruded through calibrated pore-size membranes into a flowing continuous phase that strips the droplets from the surface of the membrane (FIG. 5). The droplets were then hardened into microspheres by solvent exchange with the continuous phase, washed with additional continuous phase, and dried by lyophilization. The washing step may also include a “scalping” step whereby very small microspheres or “fines” may be further removed by selective filtration. This provides single emulsion microspheres with octreotide and PLGA.

### Scanning Electron Microscopy (SEM)

For surface images, samples were mounted to an aluminum stub using a carbon tab followed by gold sputter coating and SEM imaging.

For cross-sections, samples were mixed with Loctite epoxy and allowed to cure overnight. The samples were then frozen using liquid nitrogen and cracked using a mortar and pestle. Cracked portions were then mounted to an aluminum stub using carbon tape and colloidal graphite and sputter coated with gold.

### Size Distribution

Size distribution of microsphere populations was quantitated using automated image analysis. Cell Profiler (cellprofiler.org) software was used to find and measure microsphere diameter based on light microscopy images. The identifyPrimaryObjects module was used and calibrated with a micrometric scale. Starting with light microscopic images of microspheres, images were analyzed to automatically detect microsphere shape, fill in microsphere shapes, and measure microsphere dimensions.

Results

Without wishing to be bound to theory, it is thought that homogeneous solutions of PLGA in a mixed solvent such as methylene chloride and ethanol are limited in concentration due to electrostatic repulsion between carboxy-termini, but solutions with PLGA and compounds with positive charges (e.g., octreotide) are possible at higher concentrations of PLGA due to enhanced cohesive forces between positive charges on the peptide and negative charges on PLGA (FIG. 6A) Likewise, higher concentrations of octreotide in the mixed solvent are facilitated by the high PLGA concentration, e.g., the two solutes, PLGA and octreotide, mutually increase the solubility of one another in the mixed solvent. After extrusion of a feed through a 20 μm membrane, PLGA alone did not form acceptable microspheres on membrane emulsification, but PLGA combined with 20 mg/mL octreotide led to formation of microspheres, as imaged by light microscopy (FIG. 6B). A variety of solvents were tested for ability to dissolve octreotide and PLGA (FIG. 6C). These results demonstrated the differing solvation needs of these two molecules. Anisole, chloroform, ethyl acetate, methyl acetate, ethyl formate, dichloromethane, and acetonitrile had varying abilities to dissolve PLGA (as did mixtures of 2-butanone and anisole or ethyl acetate), and dichloromethane worked best to dissolve both the PLGA and octreotide. The best solvents for octreotide were ethanol and methanol, though ethanol has advantages as an FDA Class 3 solvent. Different PLGA polymers were also tested for microsphere formation (FIG. 6D). PLGA polymers with carboxy-termini were able to form microspheres, but ester-capped species were not, consistent with the model shown in FIG. 6A. All of the PLGA species shown in FIG. 6D can be used to make microspheres, but the species with MW: 1-5 kD, 7-17 kD, and 24-38 kD were the easiest to produce.

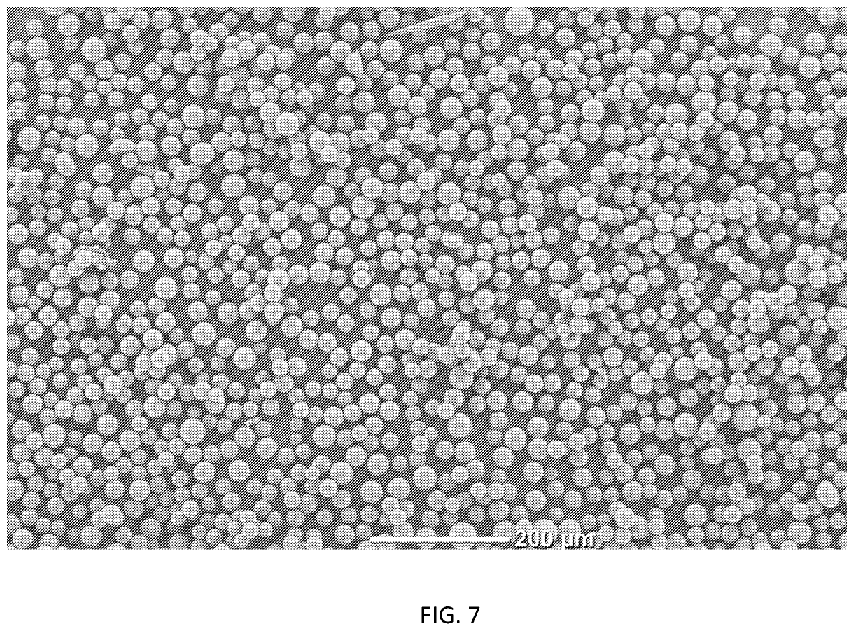

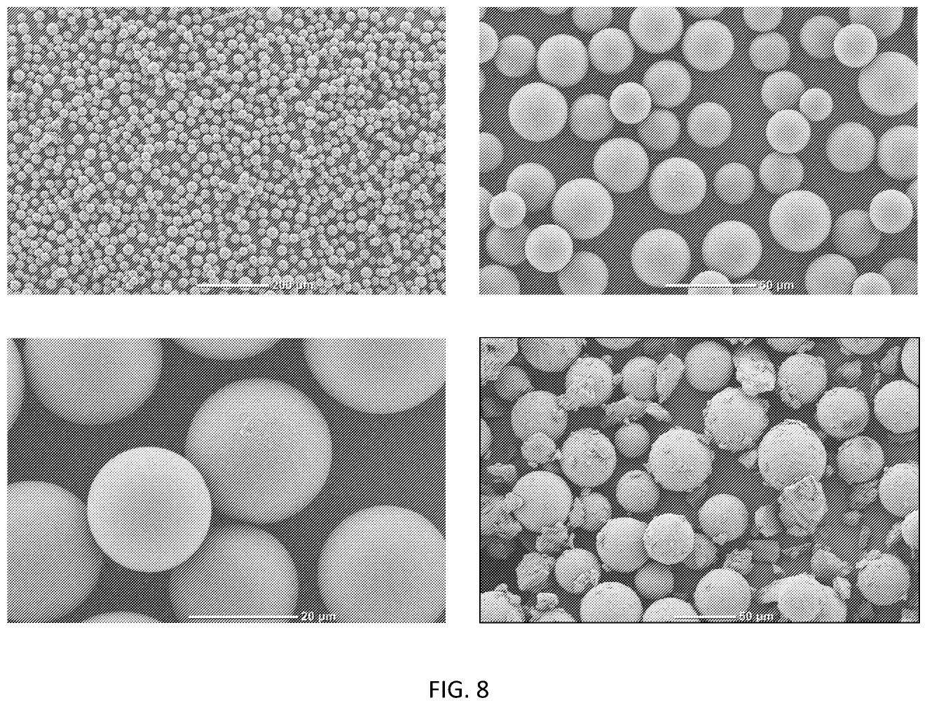

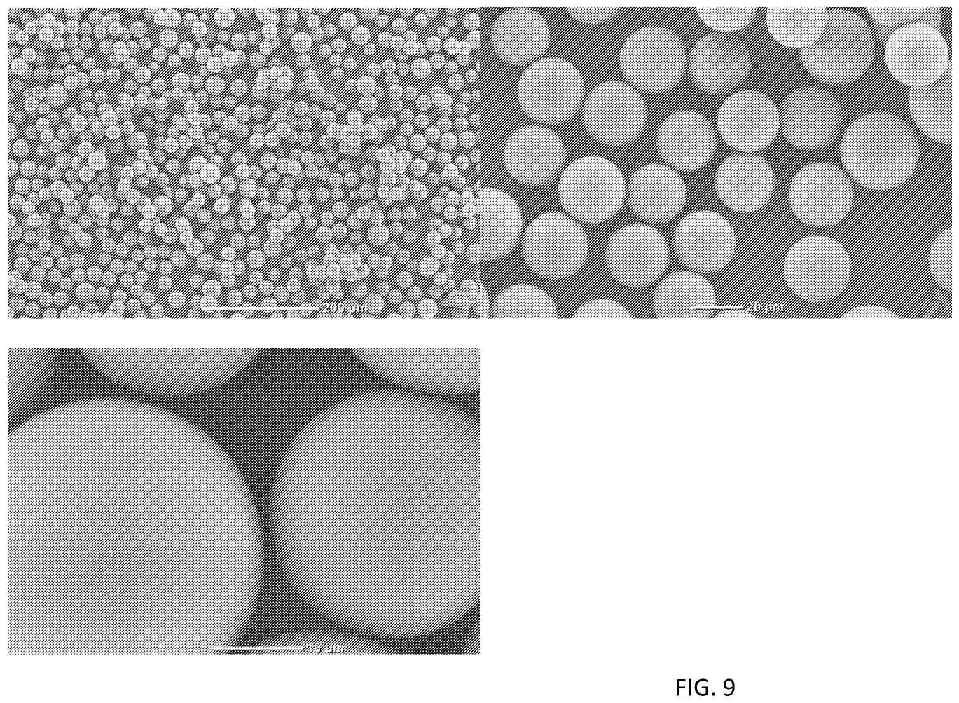

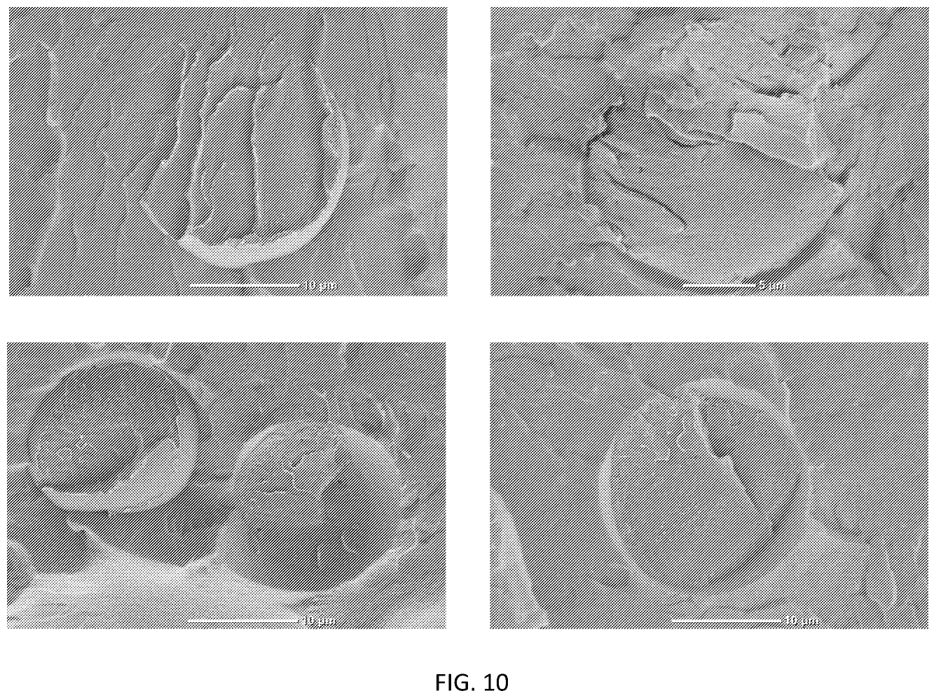

Octreotide/PLGA microspheres were manufactured according to formulation 149 as described above. SEM analysis revealed a monodisperse population of single emulsion microspheres with low surface porosity (FIG. 7). These characteristics were even more apparent when imaging the microspheres at higher magnification (FIGS. 8 & 9). Compared with SANDOSTATIN® LAR (octreotide), formulation 149 microspheres had a much smoother, more regular surface (FIG. 8, cf. top right and bottom right). Analysis of the interior of these microspheres by cryofacture SEM showed a substantially more uniform internal structure as well (FIG. 10). Without wishing to be bound to theory, it is thought that microspheres with a smoother, less rough outer surface have reduced porosity and associated burst and improved flow and handling of the dry powder microspheres.

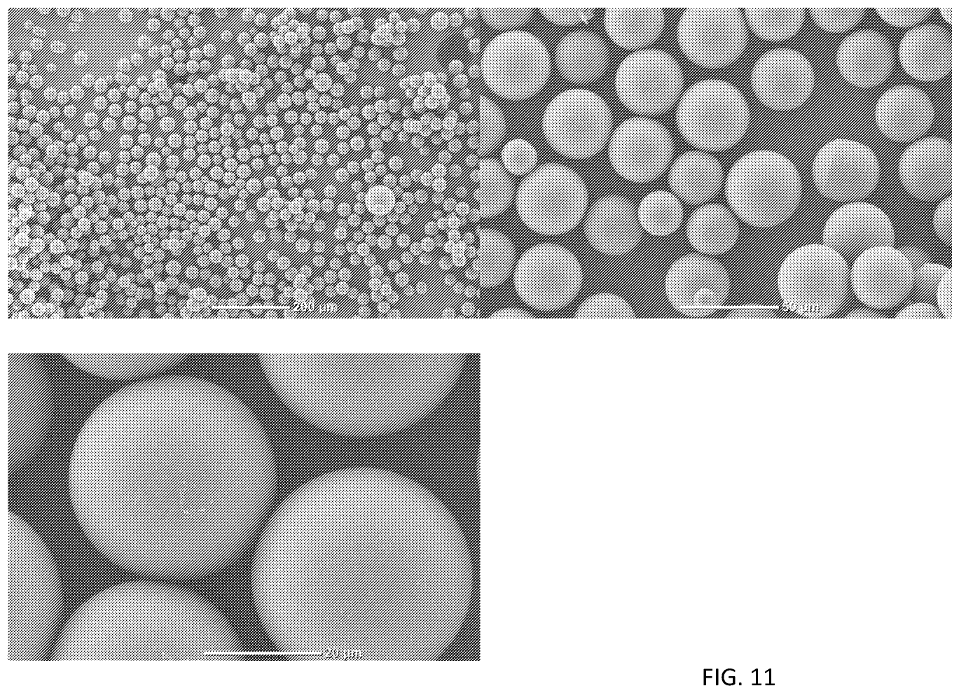

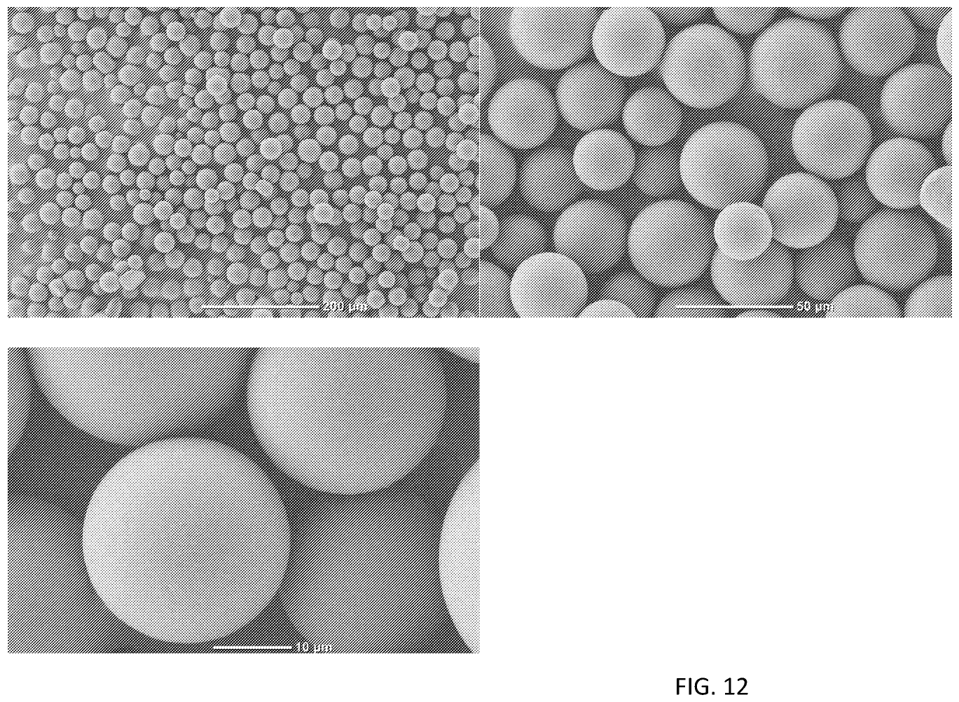

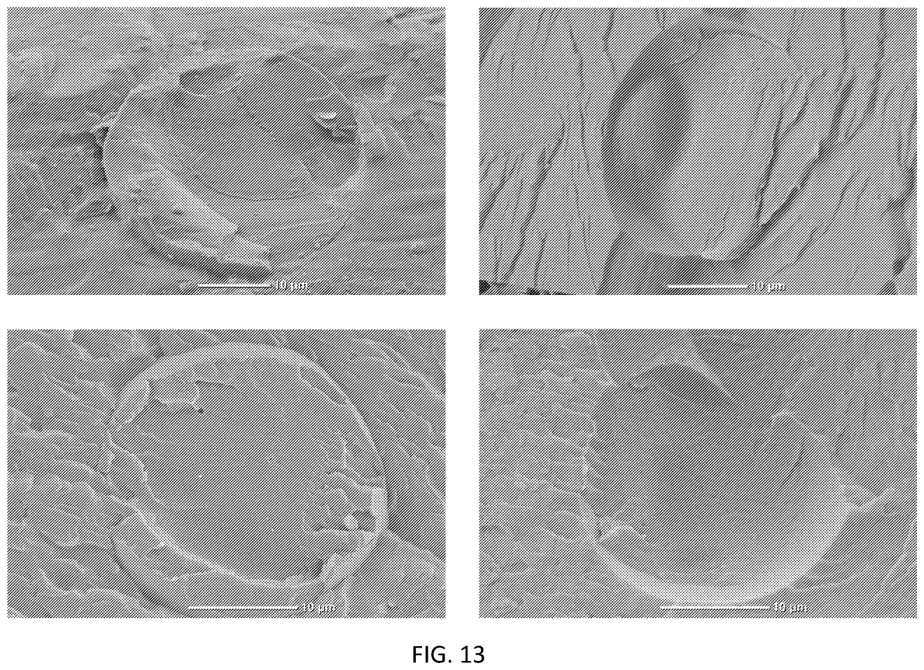

Octreotide/PLGA microspheres were also manufactured according to formulation 131 as described above. Again, SEM analysis revealed a population of microspheres with a highly regular shape and smooth surface (FIGS. 11 & 12). Cryofracture analysis of these microspheres also confirmed a comparatively uniform interior with few or no internal structures (FIG. 13).

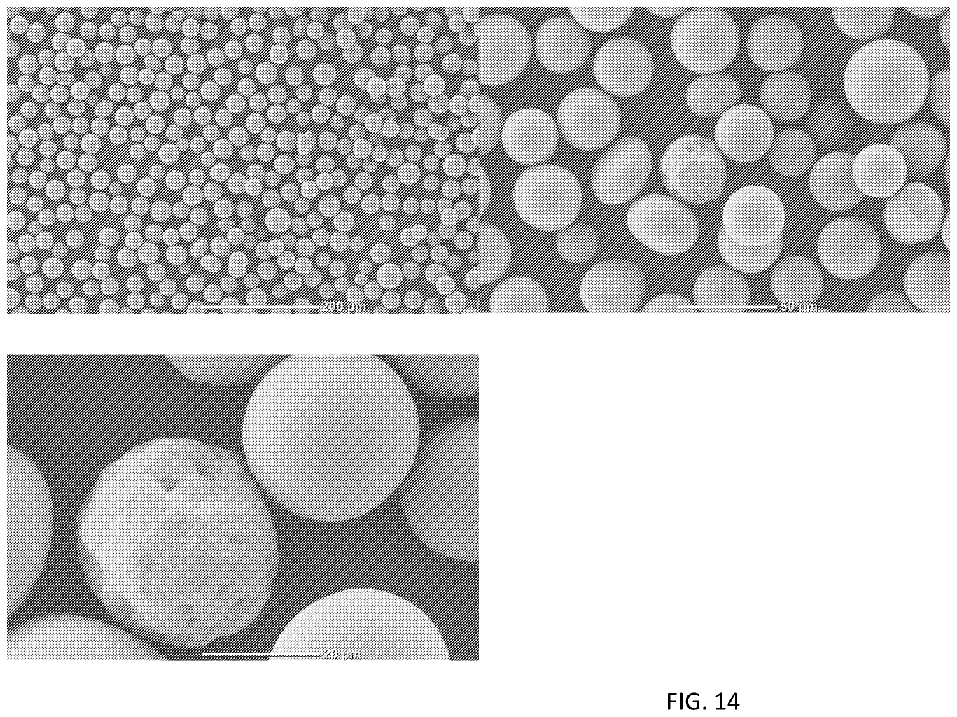

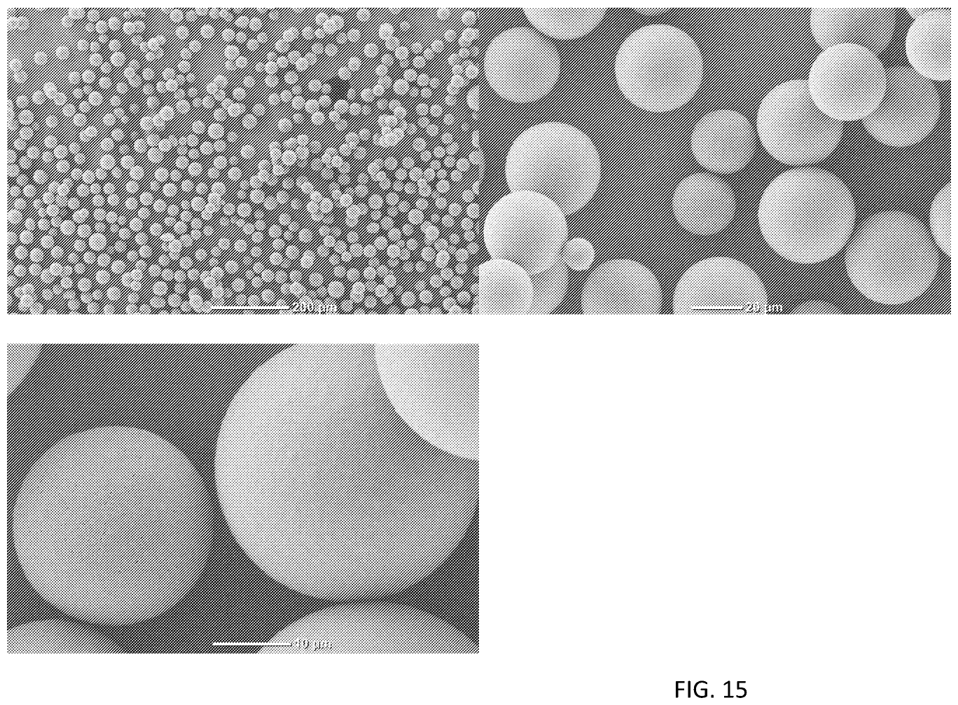

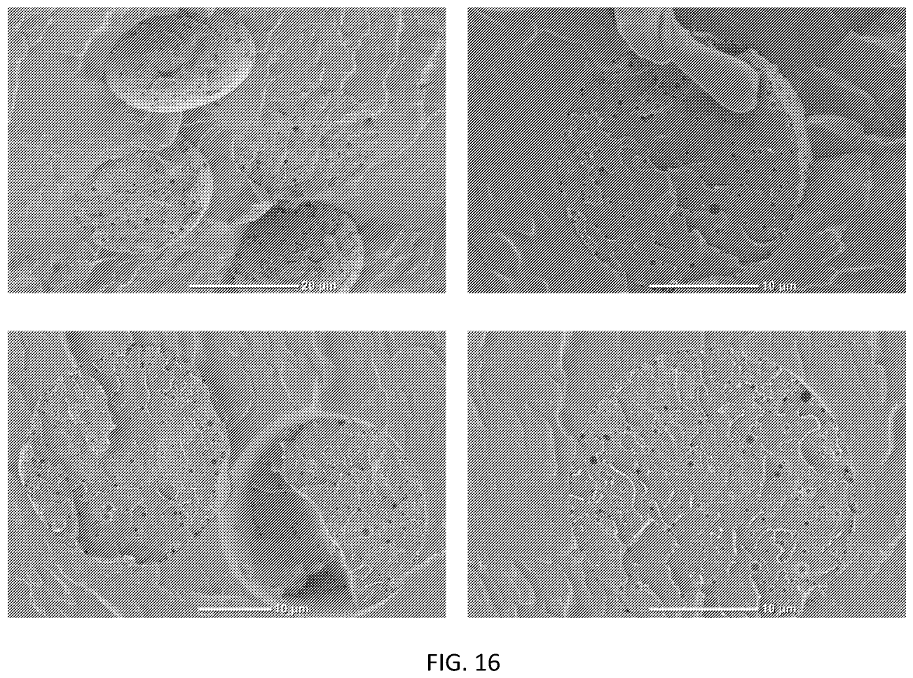

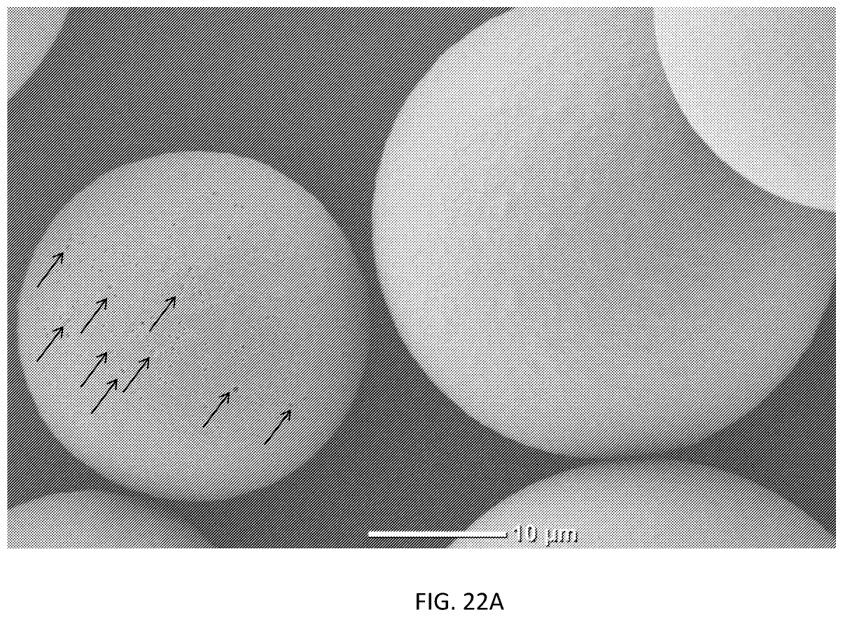

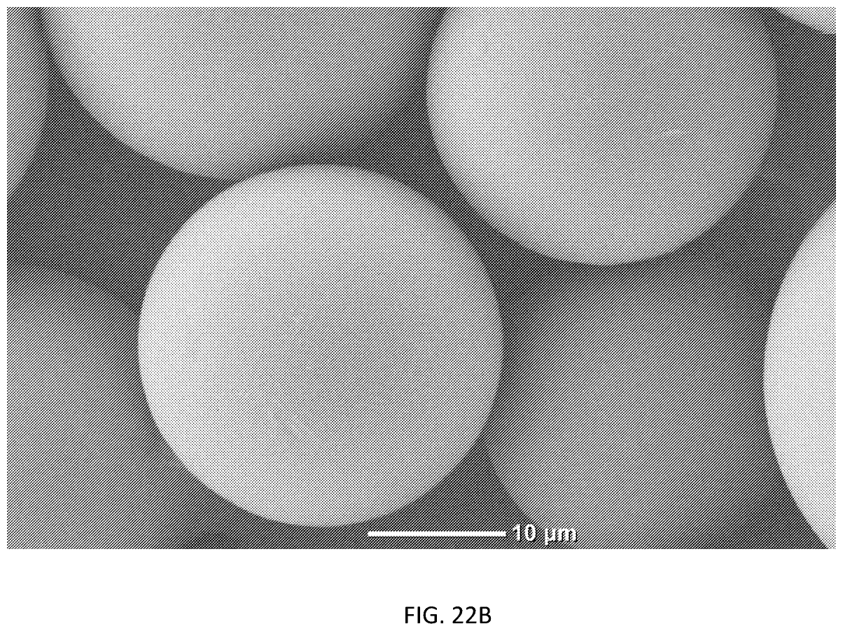

Octreotide/PLGA microspheres were also manufactured according to formulation 73, which was designed to approximate the characteristics of the SANDOSTATIN® LAR (octreotide) depot formulation. SEM analysis revealed a population of microspheres with a higher frequency of surface pores than formulations 131 and 149 though less internal structure than SANDOSTATIN® LAR (FIGS. 14 & 15; see also FIG. 22A, which shows a larger version of FIG. 15, bottom left). Cryofracture analysis of formulation 73 microspheres also demonstrated more internal porosity than microspheres made according to formulations 131 and 149 (FIG. 16). Without wishing to be bound by theory, the homogeneous feed solution and membrane emulsification may reduce porosity and internal structure versus SANDOSTATIN® LAR double emulsion process, while the addition of glycerol may further reduce the porosity and internal structure (perhaps by acting as a plasticizer).

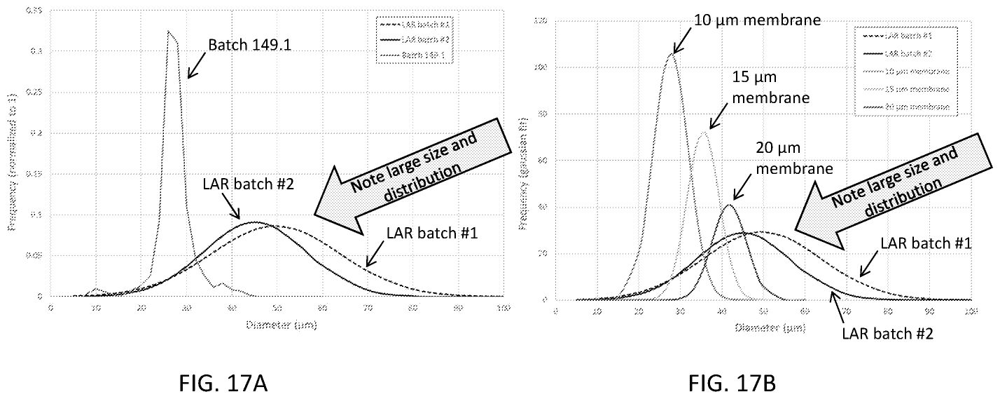

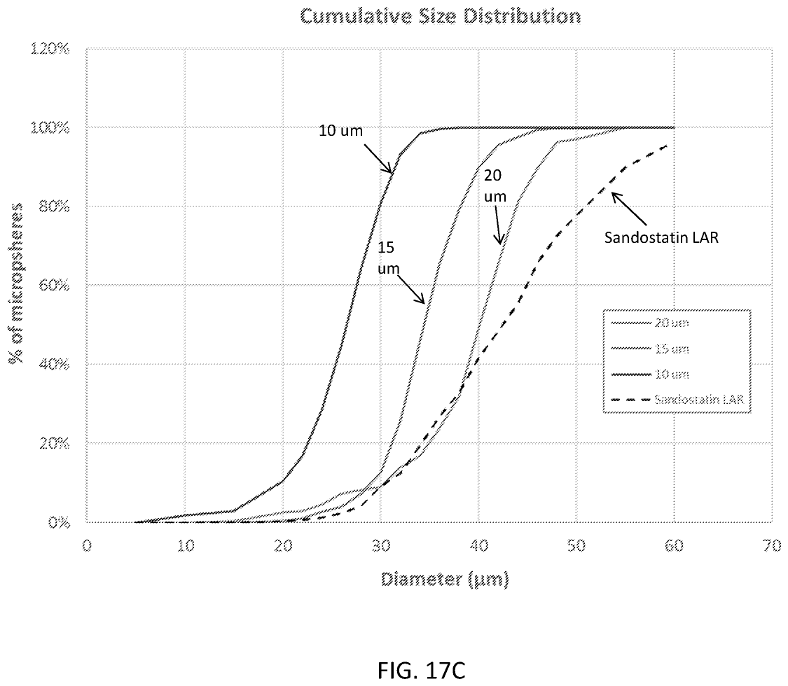

The size distributions of these microsphere populations were also analyzed quantitatively using image analysis, as described above. Two different batches of SANDOSTATIN® LAR (octreotide) were found to have broad size distributions with a median diameter in the approximate range of 40-50 μm (FIGS. 17A & 17B). In contrast, microspheres made according to formulation 149 demonstrated a much tighter size distribution with a median diameter of approximately 27-28 μm (FIG. 17A). The median microsphere diameter was dependent upon pore size of the membrane used during extrusion, with smaller pore sizes leading to smaller median diameter (FIG. 17B). Cumulative size distribution further demonstrated the smaller size and tighter distribution of these microsphere formulations, as compared to SANDOSTATIN® LAR (FIG. 17C).

These results demonstrate improved formulations of therapeutic compound (e.g., octreotide) characterized by smaller, smoother, and more regular microspheres with a narrower size distribution than existing formulations. Moreover, the use of different membrane pore sizes for extrusion allowed for exquisite control of microsphere sizes. These formulations are thought to provide improved monodispersity and allow for easier, more uniform manufacturing.

### Example 2: Improved Burst Kinetics and Loading with Therapeutic Compound-Containing Microspheres Made with Multiple Polymer Species

The formulations described in Example 1 above were characterized for their pharmacokinetic properties. These experiments demonstrated the advantageous properties of the formulations of the present disclosure.

Methods

### Formulations

Formulations with different blends of PLGA species were generated as described in Table B. All formulations contained 200 mg/mL PLGA in the feed. Two species were used in varying amounts: RESOMER® RG 502H (molecular weight: 7 kD-17 kD; Evonik Industries) and RESOMER® RG 503H (molecular weight: 24 kD-38 kD; Evonik Industries).

TABLE B
Dispersed phase composition of octreotide formulations

| | Formulation # | | | |
|---|---|---|---|---|
| | #73 | #139 | #137 | #121 |
| 502h (%) | 0 | 50 | 75 | 100 |
| 503h (%) | 100 | 50 | 25 | 0 |
| PLGA (mg/ml) | 200 | 200 | 200 | 200 |
| Octreotide (mg/ml) | 20 | 20 | 30 | 40 |

### Pharmacokinetic (PK) Studies

Above formulations were tested for PK properties in male New Zealand white rabbits. 33 male rabbits were assigned to 11 groups (3 rabbits/group). First day of dosing was day 1. Groups were administered a single dose of formulation by subcutaneous or intramuscular injection. All animals received either 85 mg of PLGA in SANDOSTATIN® LAR or 100 mg of PLGA in above formulations per injection in 1 mL dose/animal. 1 mL whole blood was collected into a tube containing K₃EDTA anti-coagulant at various time points after day 1, and plasma was used for analysis of octreotide.

HPLC with MS/MS detection was used to quantify amount of octreotide in serum samples. Briefly, octreotide-¹³C₆ was used as internal standard, and samples were processed by solid phase extraction. XSelect CSH Phenyl-Hexyl and Kinetex Biphenyl columns were used in column switching mode. Mobile phase was nebulized using heated nitrogen in a Turbo Spray (AB Sciex) source/interface, and ionized compounds were detected by MS/MS in electrospray positive mode.

### RP-HPLC Analysis of Microspheres

Drug concentration and purity of microsphere samples were tested by reverse phase high-performance liquid chromatography (RP-HPLC). A Synergi™ 4 μm Max-RP 80 Å, 250×4.6 mm column (Phenomenex) was used with gradient instrument method and run time of 40 minutes, with an elution time of approximately 17 minutes for octreotide. Mobile phase A was 0.02% (v/v) trifluoroacetic acid (TFA) in water, and mobile phase B was 100% acetonitrile (ACN). Detection was at 220 nm. USP octreotide was used as a reference standard, and octreotide trifluoroacetate salt parallel and anti-parallel dimers were also used to identify octreotide peak by retention time. To prepare microsphere samples, samples were allowed to equilibrate at room temperature for 1 hour, then 50 mg sample was transferred to a 4 mL cryo vial. 2.0 mL DMSO was then added, and vial was capped. Vortexing was used to dissolve sample. 200 μL solution was then transferred to a tube and mixed with 800 μL TFA/water. After vortexing to break up solids, tubes were centrifuged at 8000 rpm for 3 minutes, and supernatant was transferred into HPLC vial for analysis.

Results

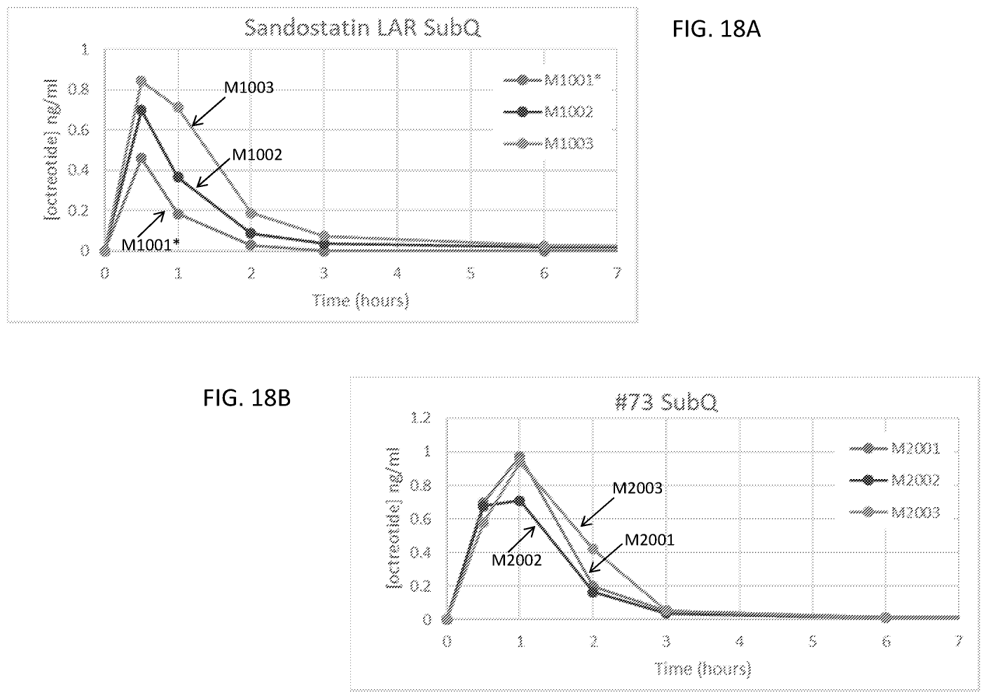

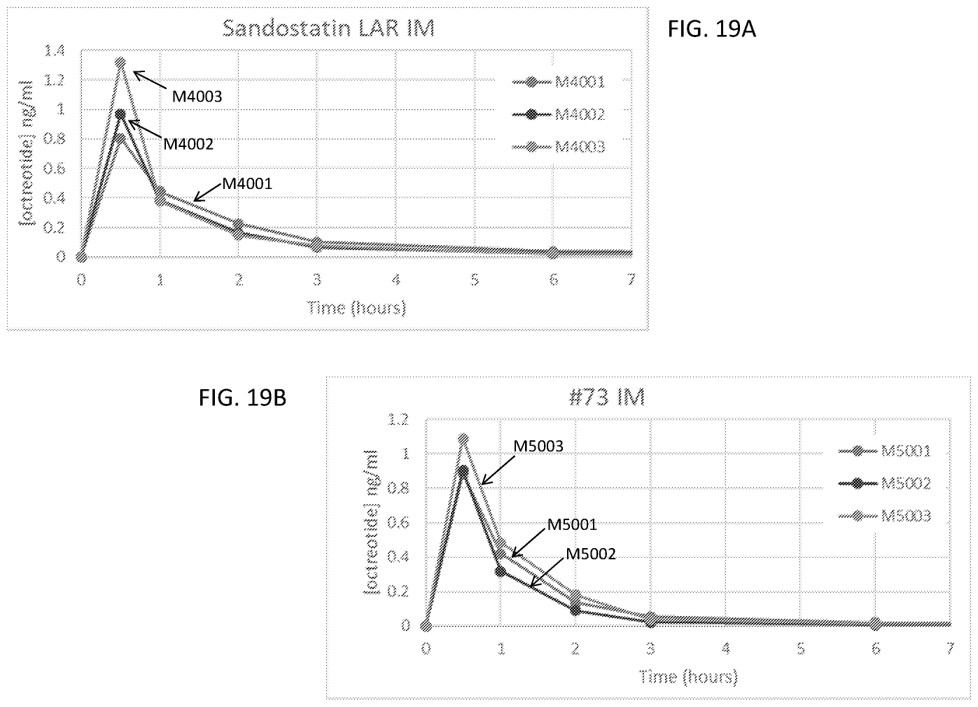

The pharmacokinetic properties of SANDOSTATIN® LAR (octreotide) and formulation 73 were compared following subcutaneous or intramuscular injection. Serum octreotide concentrations were measured after a single injection of 87 mg SANDOSTATIN® LAR (4.6 mg octreotide) or 100 mg formulation 73 (˜5 mg octreotide). Administration by subcutaneous injection resulted in very similar properties for SANDOSTATIN® LAR (FIG. 18A) and formulation 73 (FIG. 18B) during the initial burst period over three independent injections. In particular, the burst and release of octreotide were very similar between the two formulations. Intramuscular injections also yielded highly similar results for both formulations during the initial burst period (FIGS. 19A & 19B). Small differences were observed between subcutaneous and intramuscular injection, such as a faster release and slightly higher burst Cmax for intramuscular, but overall both administration routes yielded generally similar bursts. In both routes, formulation 73 had very similar burst properties as compared with SANDOSTATIN® LAR.

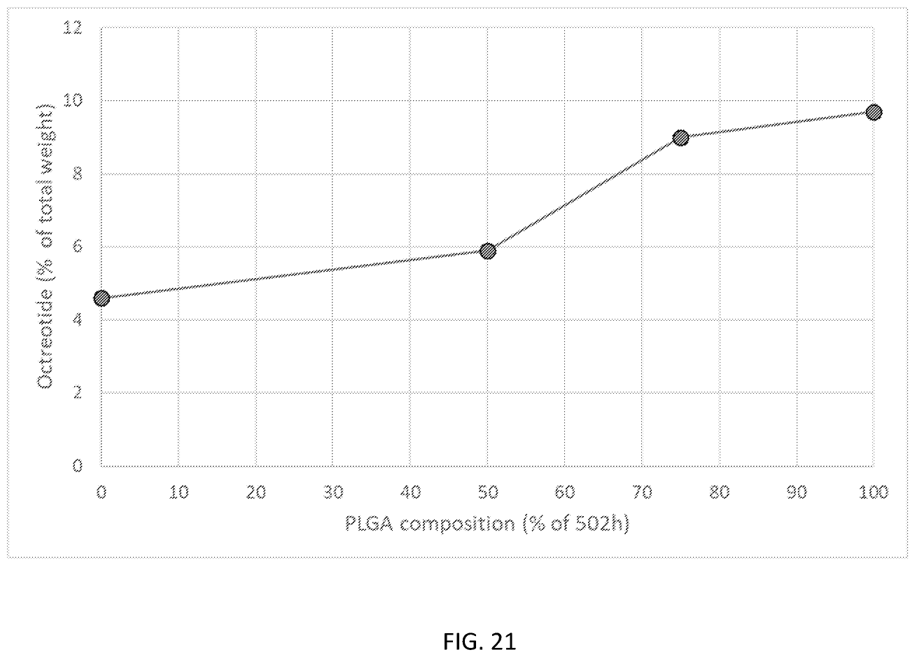

Formulations 121, 137, and 139 were compared to formulation 73 (which mimics loading of SANDOSTATIN® LAR as demonstrated above) in order to ascertain the effect of blending different polymer species (in this case, different molecular weights of PLGA polymers). The overall amount of PLGA was kept constant, but the precise blend of different species was varied (see Tables A and B). Burst Cmax and burst AUC (time 0 to 3 hours) were then analyzed for different PLGA blends by normalizing the results per mg of injected octreotide (FIG. 20). In addition, the amount of octreotide (as percentage of total weight) vs. the PLGA composition (percentage of PLGA MW: 7 kD-17 kD) was plotted (FIG. 21).

These results indicated that increasing the ratio of PLGA (MW: 7 kD-17 kD) to PLGA (MW: 24 kD-38 kD) polymer in the microspheres increased compound loading (i.e., percentage of octreotide in the microsphere; see FIG. 21) but kept the in vivo burst per drug content fairly constant. As a result, the octreotide burst per mass of peptide decreased (see burst Cmax in FIG. 20) or stayed constant (see burst AUC in FIG. 20) for blends with higher content of the lower molecular weight PLGA species. However, this trend stopped at 75%-100% PLGA MW: 7 kD-17 kD, where a pronounced burst was observed (see formulation 121 in FIG. 20).

In conclusion, blending different species of PLGA led to formulations with higher loading of therapeutic compound with a proportional burst AUC and reduced burst Cmax. Unexpectedly, higher loading may be obtained at no burst penalty until 75%-100% PLGA MW: 7 kD-17 kD, thereby providing more patient-friendly injections of less material at compositions of 75% or less of PLGA MW: 7 kD-17 kD with no increase in burst exposure.

### Example 3: Glycerol-Containing Formulations of Therapeutic Compounds into Microspheres with Improved Physical and Pharmacokinetic Properties

The effect of glycerol on the physical pharmacokinetic properties of microsphere formulations was explored.

Methods

For degradation studies, formulations 73, 131, 121, and 132 were tested. As described above, formulation 73 was prepared with 200 mg/mL RESOMER® RG 503H (molecular weight: 24 kD-38 kD; Evonik Industries) and 20 mg/mL octreotide; formulation 131 was prepared with 200 mg/mL RESOMER® RG 503H (molecular weight: 24 kD-38 kD; Evonik Industries), 20 mg/mL octreotide, and 0.9 mg/mL glycerol; formulation 121 was prepared with 200 mg/mL RESOMER® RG 502H (molecular weight: 7 kD-17 kD; Evonik Industries) and 40 mg/mL octreotide; and formulation 132 was prepared with 200 mg/mL RESOMER® RG 502H (molecular weight: 7 kD-17 kD; Evonik Industries), 40 mg/mL octreotide, and 0.9 mg/mL glycerol. Microsphere samples were dissolved at room temperature and tested for degradation products by mass spectrometry (degradation product peaks were quantified as percentage of the main peak) after the indicated time period at 5° C. or 40° C.

Results

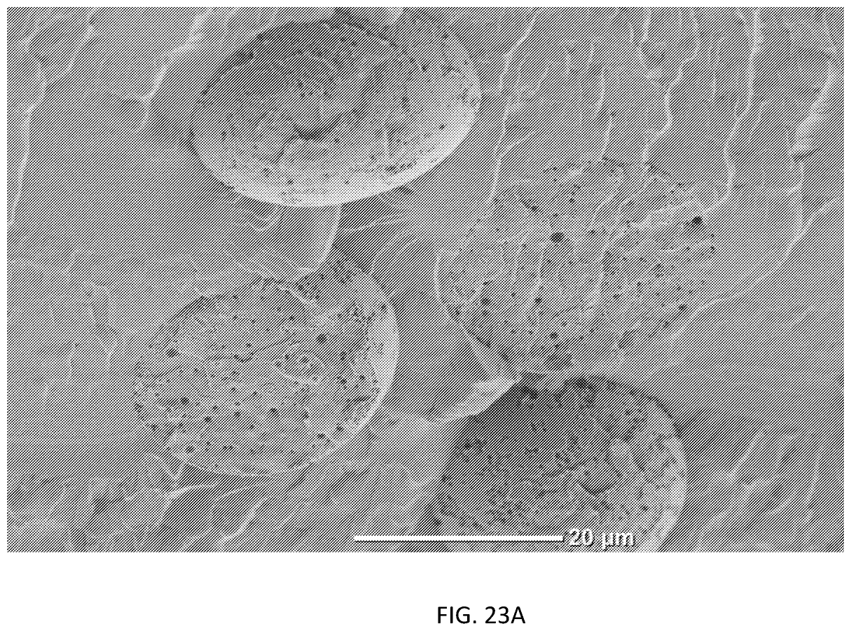

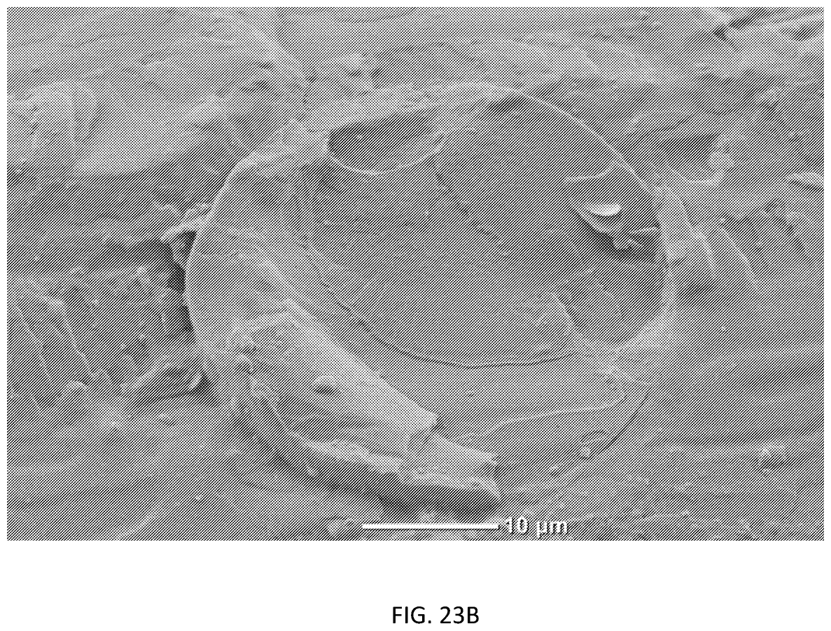

Microspheres made according to formulations 131 and 149 as described in Example 1 were found to have reduced surface pores as compared to those made according to formulation 73. This was demonstrated by SEM surface imaging (cf. FIGS. 22A & 22B). FIG. 22A shows microspheres according to formulation 73 with a number of surface pores (arrows), whereas the microspheres according to formulation 131 shown in FIG. 22B have a more smooth and uniform surface. These surface differences were also imaged by cryofracture EM, which showed a large number of empty internal structures for microspheres according to formulation 73 (FIG. 23A) that were not present in microspheres according to formulation 131 (FIG. 23B).

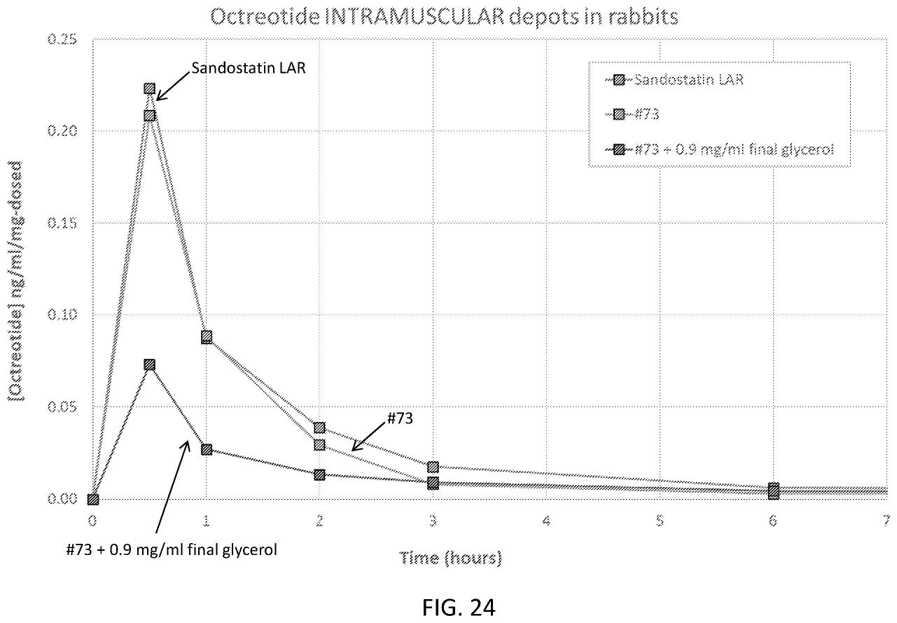

Given these differences in physical microsphere properties, the effect of glycerol on the pharmacokinetic properties of octreotide microspheres was examined. Intramuscular injections of SANDOSTATIN® LAR and formulation 73 microspheres had nearly identical burst and release kinetics (FIG. 24). However, the addition of 0.9 mg/mL glycerol (final concentration subjected to extrusion) markedly reduced the octreotide burst of formulation 73. This demonstrates a significant benefit in burst reduction through incorporation of glycerol into microsphere formulations. Without wishing to be bound to theory, it is thought that pores can lead to cavities in the interior of the microspheres, increasing accessibility and promoting compound burst.

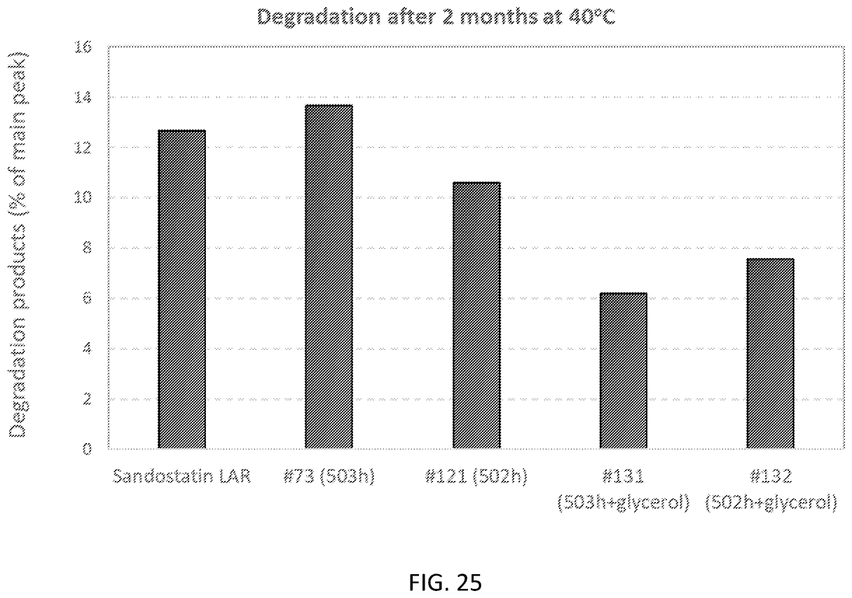

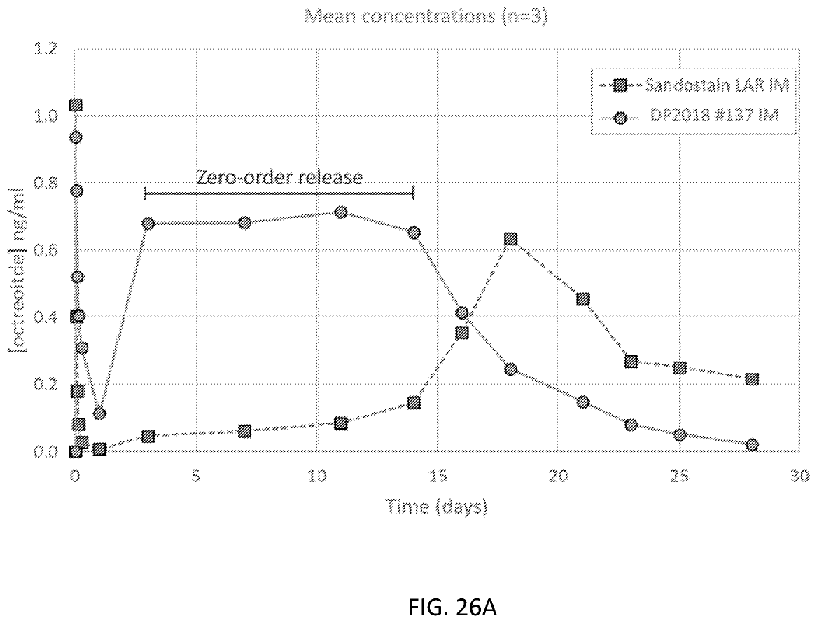

Incorporation of glycerol was also found to reduce therapeutic compound/salt degradation. SANDOSTATIN® LAR and formulation 73 microspheres had similar proportions of octreotide degradation products after 2 months at 40° C. (FIG. 25). However, microspheres according to formulations 131 and 132 (containing glycerol and PLGA MW: 24 kD-38 kD or PLGA MW: 7 kD-17 kD, respectively) showed an approximately 50% reduction in degradation products under the same conditions. These results are shown in Tables C and D.

TABLE C
Stability results for samples kept at 5°C.

| | Sandostatin | | | | | | | | | |
|---|---|---|---|---|---|---|---|---|---|---|
| | LAR | | 73 | | 121 | | 131 | | 132 | |
| | | Total | | Total | | Total | | Total | | Total |
| Time | Potency | % | Potency | % | Potency | % | Potency | % | Potency | % |
| (Month) | (mg/mL) | Imp | (mg/mL) | Imp | (mg/mL) | Imp | (mg/mL) | Imp | (mg/mL) | Imp |
| 3 | 4.5 | 4.31 | 5.7 | 3.89 | 11.3 | 5.26 | 4.0 | 1.16 | 11.0 | 3.71 |

TABLE D
Stability results for samples kept at 40° C.

| | Sandostatin | | | | | | | | | |
|---|---|---|---|---|---|---|---|---|---|---|
| | LAR | | 73 | | 121 | | 131 | | 132 | |
| | | Total | | Total | | Total | | Total | | Total |
| Time | Potency | % | Potency | % | Potency | % | Potency | % | Potency | % |
| (Month) | (mg/mL) | Imp | (mg/mL) | Imp | (mg/mL) | Imp | (mg/mL) | Imp | (mg/mL) | Imp |
| 0.5 | 3.9 | 5.88 | 5.0 | 7.13 | 10.1 | 7.23 | 3.6 | 2.74 | 9.7 | 4.63 |
| 1 | 3.5 | 8.25 | 3.6 | 9.17 | 9.5 | 9.86 | 3.0 | 4.77 | 9.4 | 7.17 |
| 2 | 3.4 | 12.66 | 4.1 | 13.67 | 9.5 | 10.60 | 3.0 | 6.20 | 9.4 | 7.57 |
| 3 | 3.4 | 19.58 | 4.2 | 19.89 | 9.0 | 16.99 | 3.1 | 10.06 | 9.5 | 11.03 |

Taken together, these results demonstrate favorable physical and pharmacokinetic properties of microspheres with glycerol (e.g., reduced burst and degradation).

### Example 4: Measurement of Microsphere Porosity

In addition to SEM imaging of microsphere surfaces, microsphere porosity is quantified by measurement of specific surface area by the Brunaer-Emmett-Teller (BET) theory. Increased porosity leads to higher specific surface area. Pores are analyzed by exposure to gases that physically bind at the solid surface through physisorption. The following gases and their respective boiling temperatures are used: N₂ at 77K, Ar at 87K, and CO₂ at 273K. For example, a 40 point N₂ physisosorption isotherm analysis can be used, providing a BET surface area and pore size distribution in the approximate range of 2-300 nm. Gas adsorption leads to formation of a gas monolayer around the microsphere surface area, then pore filling. BET surface area is then determined by adsorption isotherm plot. Micropores are indicated by a large and steep increase of isotherm, then subsequent plateau. Realistic pore-filling modeling is used to extract pore size information from the adsorption isotherm. This allows calculation of non-local density function theory (NLDFT) pore-size distribution for both micropores (e.g., pores having an internal diameter of less than 2 nm) and mesopores (e.g., pores having an internal width of 2-50 nm) as well as pore volume. Micropore measurement instrument such as Micromeritics Tristar II 3020 is used.

### Example 5: Additional Pharmacokinetic Analyses of Therapeutic Compound-Containing Microspheres

Due to the favorable biophysical and pharmacological properties of the microspheres described above, additional PK studies were undertaken to examine therapeutic compound release over longer timescales in two animal PK models, the rabbit and minipig.

Methods

Rabbit PK studies were carried out as described in Example 2, except that SANDOSTATIN® LAR depot was administered via intramuscular (IM) injection, and the formulations of the present disclosure were administered both via subcutaneous injection (SC) and via intramuscular (IM) injection.

For minipig studies, commercial SANDOSTATIN® LAR was reconstituted and injected IM into the rear limb per the manufacturer's instructions. Minipigs were injected either with subcutaneous doses of formulation 175 (37 mg total octreotide administered as four 9.4 mg doses) or an intramuscular dose of SANDOSTATIN® LAR depot (30 mg total octreotide). Formulations of the present disclosure were injected SC into the flank region in 4 different sites with an amount corresponding to a weekly dose, such that the sum of the 4 injections would approximate the AUC of the SANDOSTATIN® LAR injection. All injections were reconstituted with 1 mL diluent (using PBS for the formulations of the present disclosure and the manufacturer's supplied diluent for SANDOSTATIN® LAR). Plasma concentrations of octreotide were measured at various times post-dose. Two minipigs were used for each condition.

Results

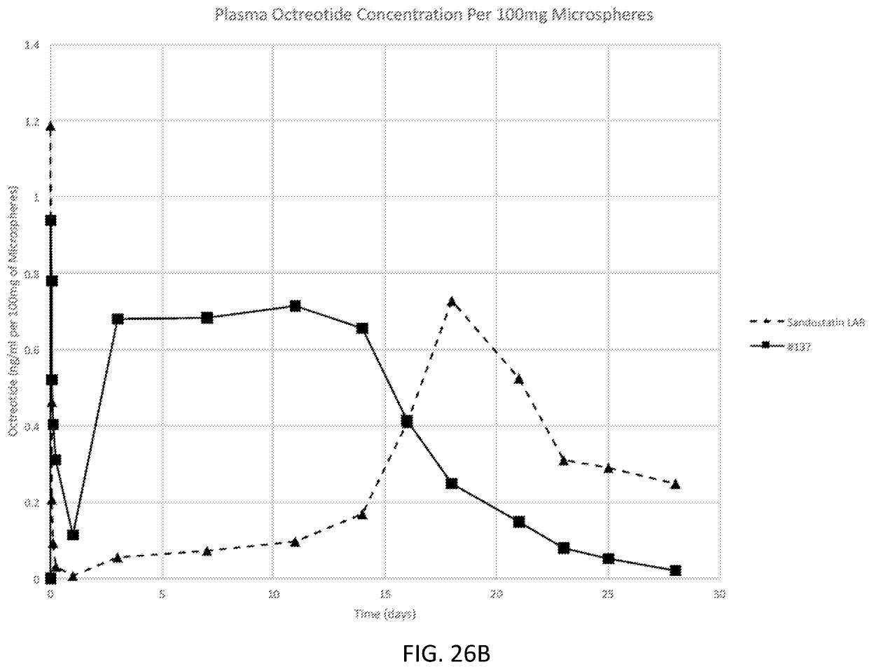

FIG. 26A compares the plasma octreotide concentrations in rabbits injected with a single intramuscular dose of 87 mg SANDOSTATIN® LAR depot or 100 mg formulation 137 (weights referring to the weight of microspheres injected, i.e., PLGA plus octreotide, not inclusive of excipients such as mannitol or diluent). Compared with SANDOSTATIN® LAR depot, formulation 137 showed an improved drug release profile with dramatically better PK. To provide a true head-to-head comparison with SANDOSTATIN® LAR depot, FIG. 26B presents the same results as shown in FIG. 26A, but with the SANDOSTATIN® LAR depot values scaled to be equivalent to 100 mg injected (matching formulation 137). These results confirm the improved PK properties of formulation 137 as compared to SANDOSTATIN® LAR depot.

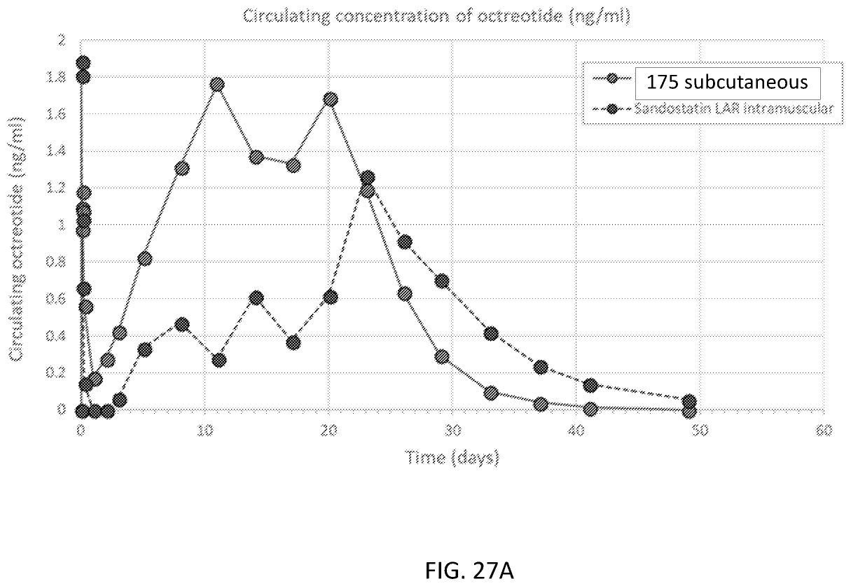

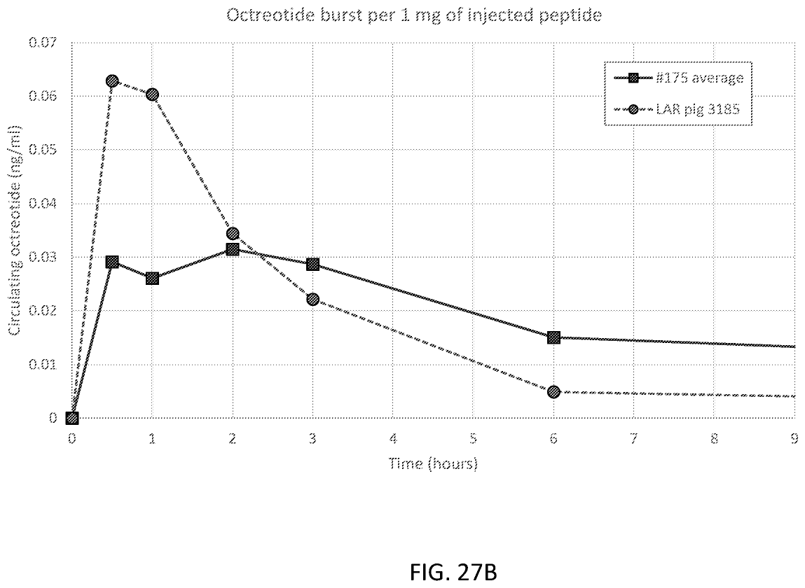

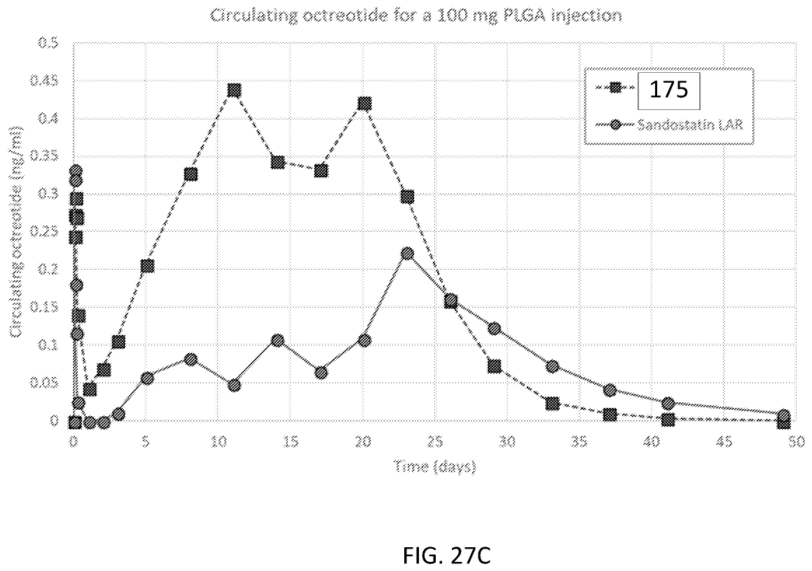

The average PK values from the minipig study are plotted in FIG. 27A. These results demonstrate that administration of formulation 175 resulted in earlier octreotide release with higher AUC, as compared with administration of SANDOSTATIN® LAR depot. In order to illustrate octreotide burst kinetics, time points within 6 hours of administration are shown in FIG. 27B with values averaged and normalized to octreotide burst per 1 mg of injected peptide. This plot clearly illustrates the more favorable burst kinetics of formulation 175. FIG. 27C shows the average levels of circulating octreotide for each group normalized to 100 mg of injected PLGA. Taken together, these results demonstrate that formulation 175 resulted in favorable burst kinetics as compared with SANDOSTATIN® LAR depot, and the pharmacokinetic properties of formulation 175 were similar to those demonstrated in the rabbit PK experiments described above.

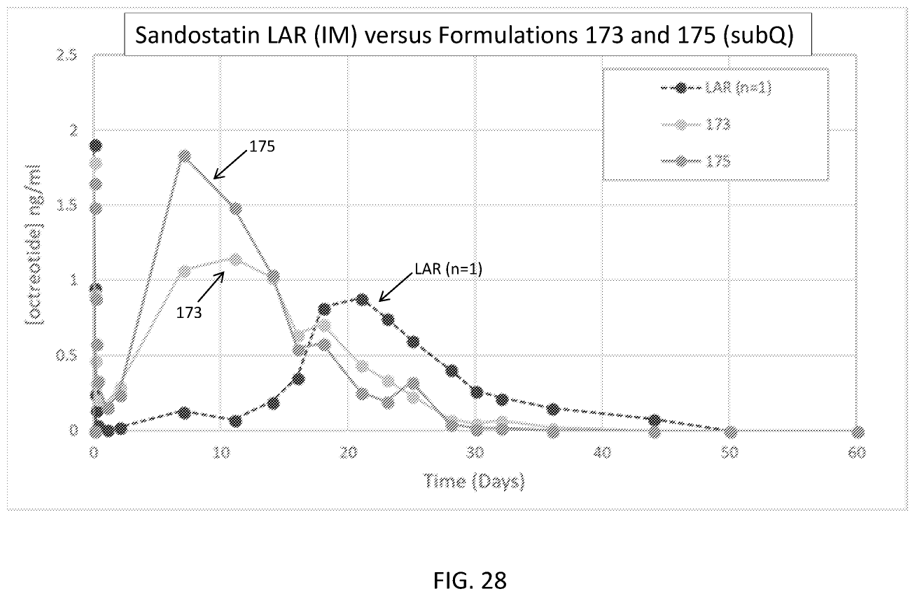

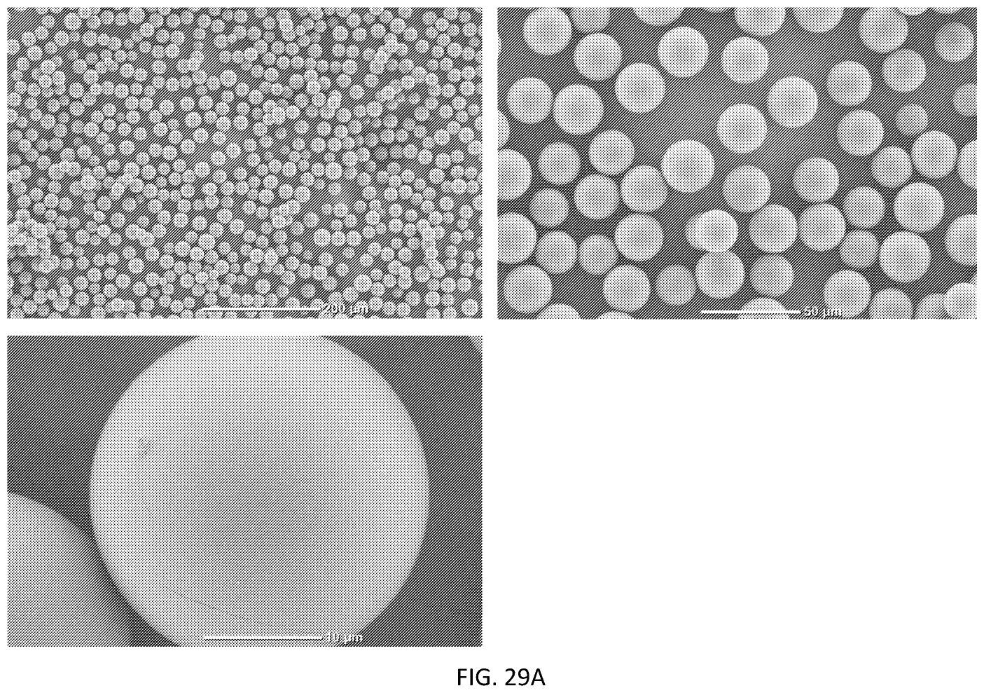

In another rabbit study, intramuscular administration of 87 mg SANDOSTATIN® LAR depot was compared against subcutaneous administration of 100 mg of formulation 173 or 175 (FIG. 28). Taken together, these results confirmed that formulations 173 and 175 led to more favorable burst kinetics and octreotide release as compared with SANDOSTATIN® LAR depot.

### Example 6: Effect of Buffer on Microsphere Properties

This Example demonstrates the effect of continuous phase buffer on microsphere properties, e.g., porosity.

Methods

Microspheres were produced as described in Example 1. The formulation included 200 mg/mL PLGA (75%:25% 502H:503H PLGA in DCM), 30 mg/mL octreotide, and 0.9 mg/mL glycerol. This same dispersed phase formulation was used for all experiments, and only the continuous phase was varied. The following continuous phases were tested:

“Glycylglycine”—100 mM glycylglycine pH8.0, 1% PVA, 100% DCM saturation

“Tris”—100 mM Tris/acetic acid pH8.0, 1% PVA, 100% DCM saturation

Results

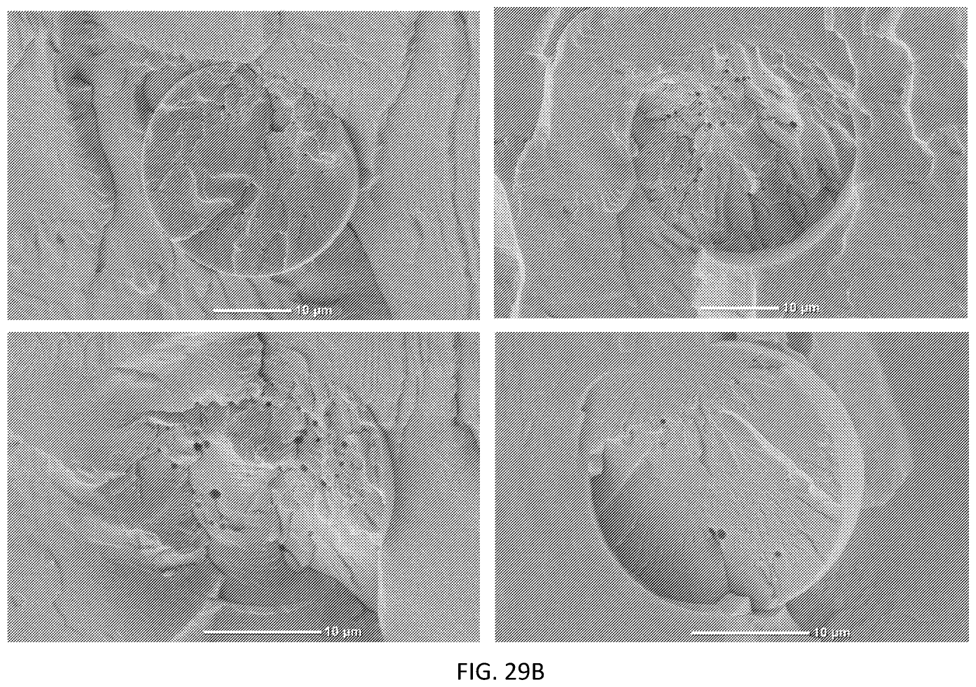

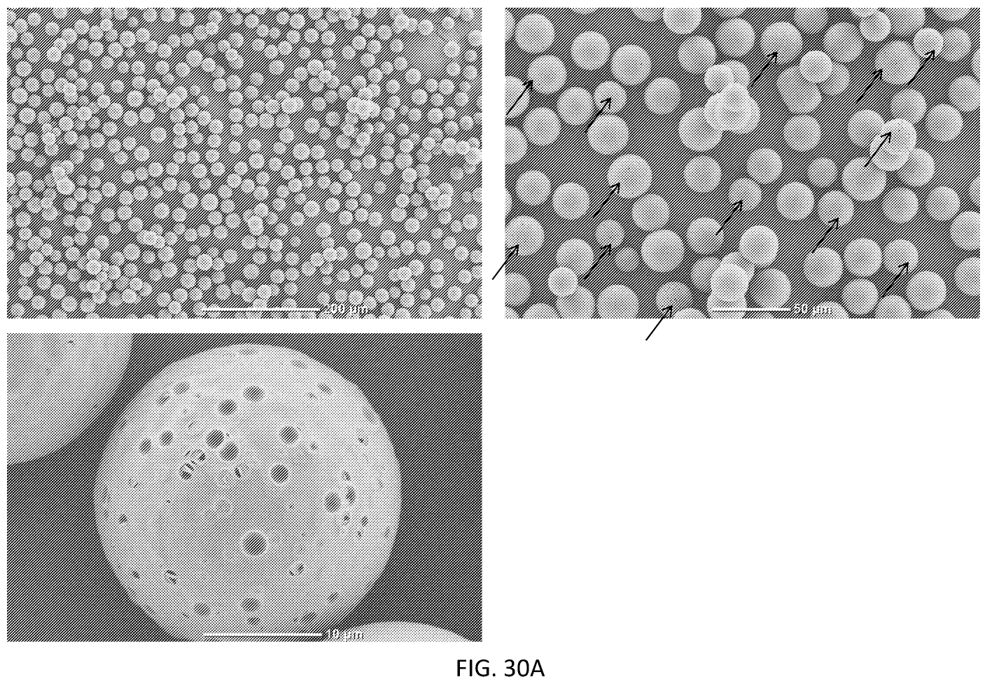

The effect of continuous phase buffer on microsphere properties was tested. Using glycylglycine buffer as described above led to production of microspheres with a uniform surface and low surface porosity (FIG. 29A). Cryofracture analysis also demonstrated low internal porosity (FIG. 29B). These results show the advantages of using a glycylglycine-based buffer (e.g., 100 mM glycylglycine pH8.0, 1% PVA, 100% DCM saturation) in the continuous phase for microsphere production.

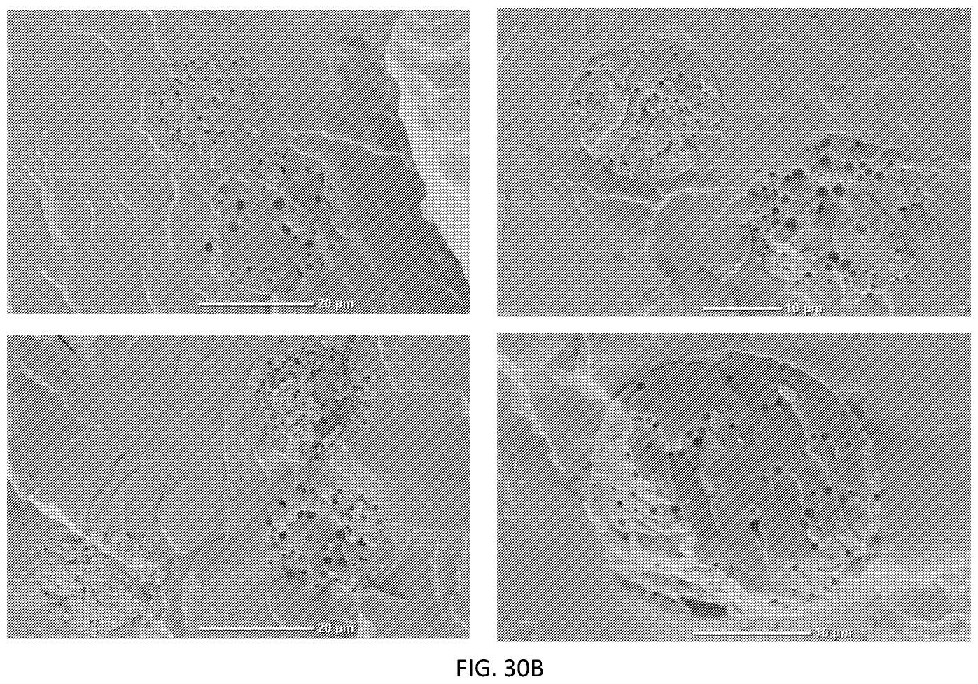

In contrast, using Tris buffer produced batches with an increased number of microspheres having greater surface porosity (FIG. 30A; see, e.g., microspheres labeled with arrows in upper right), as compared to the batch made with glycylglycine buffer in the continuous phase. Cryofracture analysis also revealed a higher amount of internal porosity as well (FIG. 30B).

These results suggest that not all amino acid-based buffers are suitable for use in the continuous phase. For example, buffers such as glycylglycine, bicine, and tricine can produce microspheres with low porosity, whereas Tris buffer leads to highly porous microspheres.

### Example 7: Microsphere Hardening and Wash Conditions

This Example describes testing the effects of pH and washing conditions on microspheres prior to lyophilization in order to reduce product-related and solvent impurities while maximizing loading.

Methods

Microspheres were produced as described in Example 1. The following continuous phases were used: 200 mM glycine pH9.0, 0.5% PVA, 100% DCM saturation and 100 mM glycylglycine (varying pH from 7.5-8.5), 1% PVA, 100% DCM saturation. Impurities and octreotide loading were monitored over time after extrusion.

Results

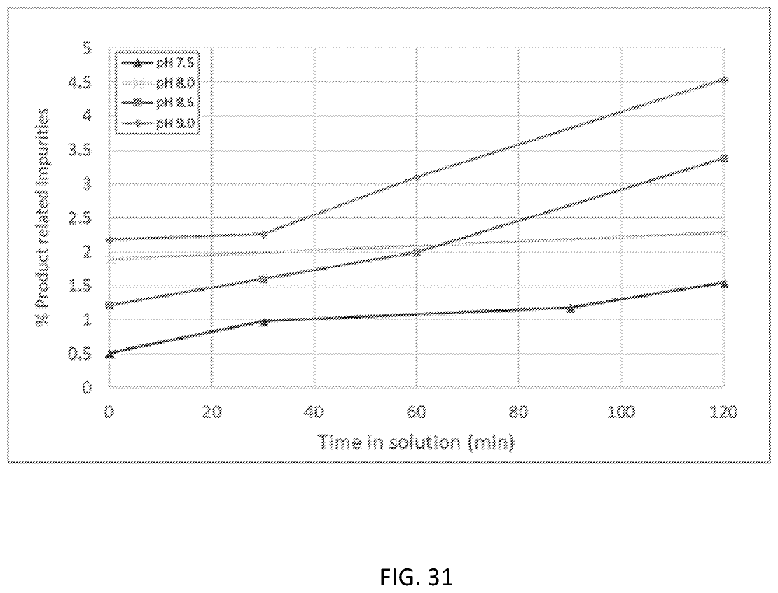

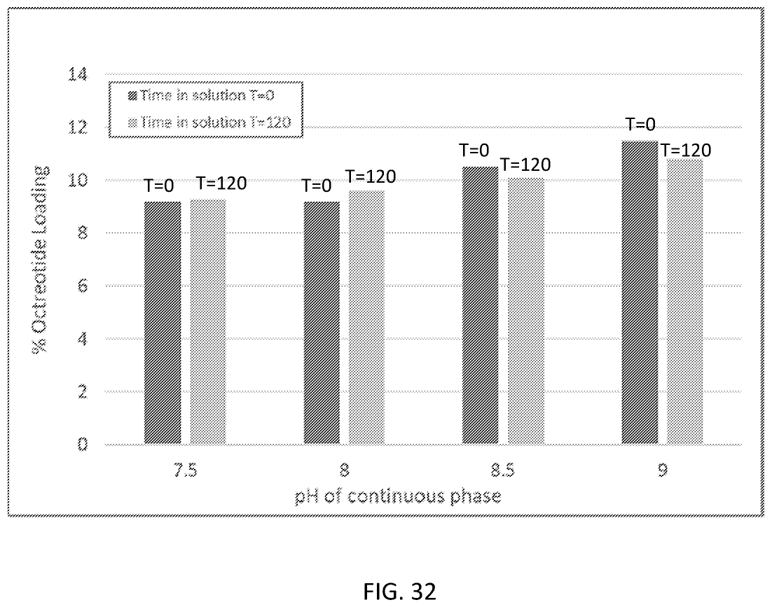

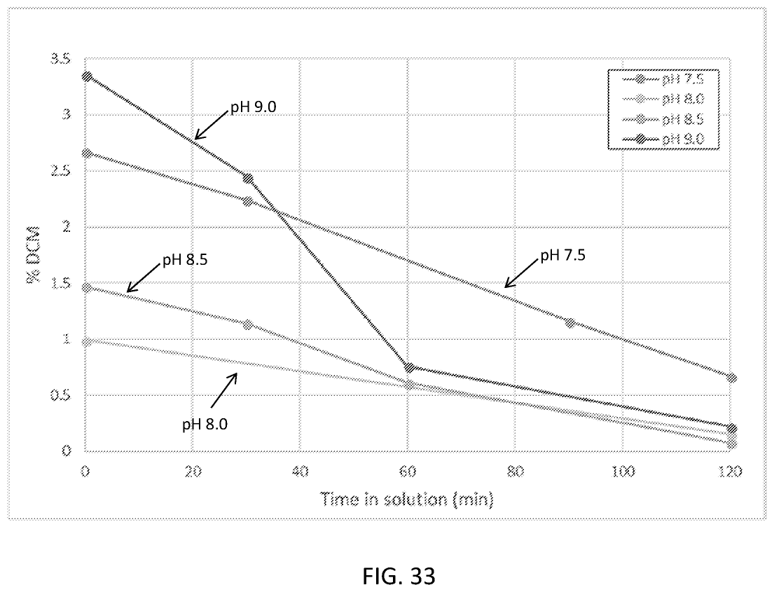

The pH of the continuous phase and hardening solution was varied from pH 7.5 to 9.0. For pH 7.5 to 8.5, glycylglycine was used as a buffer (pKa=8.25), and for pH 9.0 buffer, glycine was used to buffer the solution (pKa=9.78). After the microsphere extrusion, the microspheres were allowed to harden and product related impurities were quantified via RP-HPLC. In FIG. 31, the product related impurities were monitored over time in relation to pH. The microspheres were more stable at lower pH with lower product related impurities over time. Above pH 8.5, the product related impurities increased significantly after 30-60 mins. FIG. 32 monitors the level of octreotide loading within the microspheres. As the microspheres spent more time in solution, the level of octreotide did not change. Octreotide loading was slightly better at higher pH (above 10%); however product related impurities were significantly higher at pH above 8.5. Octreotide loading at pH 7.5 and 8.0 were also high levels at 8.5-9% and also had low product impurity levels. FIG. 33 monitors the residual DCM levels throughout the hardening stage. At pH 8.0-9.0, 120 mins was sufficient enough to bring the DCM levels into an acceptable range.

Considering the levels of impurities generated and amount of octreotide loading, the continuous phase and hardening solution at pH 8.0 using a glycylglycine buffer was the most favorable condition among those tested. Allowing microspheres to harden for 120 mins was able to maximize DCM removal while minimizing product related impurities.

### Example 8: Effect of Batch Size and Flow Rates Microsphere Manufacturing

This Example describes a reproducible and scalable process to manufacture microspheres. The effects of batch size, continuous phase flow rate, and dispersed phase flow rate were also examined.

Methods

Microspheres were produced as described in Example 1. The dispersed phase was prepared with 30 mg/mL octreotide acetate, 0.9 mg/mL glycerol with 150 mg/mL 502H, 50 mg/mL 503H PLGA dissolved in DCM. The continuous phase was prepared as 100 mM glycylglycine pH 8.0, 1% PVA, and saturated with DCM. A 10 μm membrane was used.

For a 10 gram batch, 50 mL of dispersed phase was used at a rate of 10 mL/min, with the flow rate of the continuous phase at 3.4 L/min. For a 30 gram batch, 150 mL of dispersed phase was used at a rate of 10 mL/min, with the flow rate of the continuous phase at 3.4 L/min. Microspheres were allowed to harden, washed with water, and dried. Dried microspheres were sized by quantitative image analysis of light field microscopic images (Cell Profiler).

Results

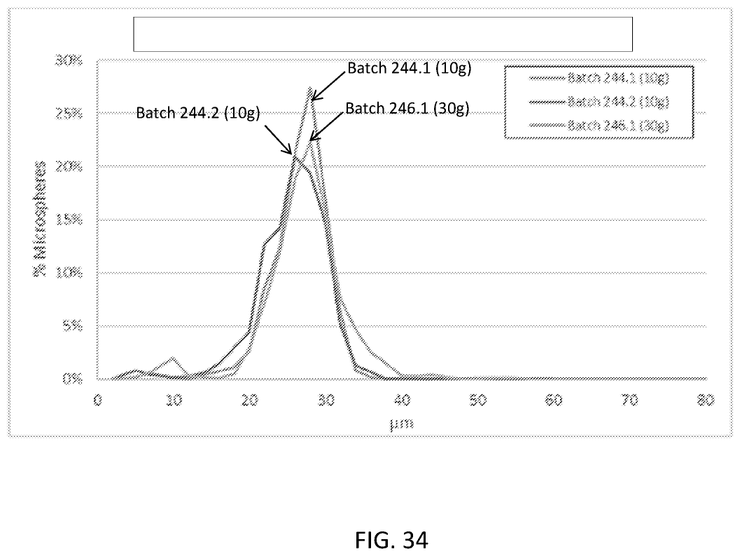

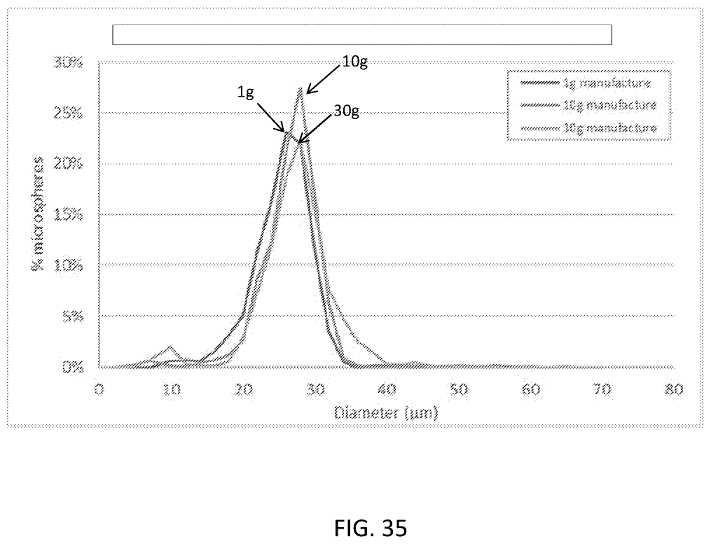

As shown in FIG. 34, two 10-gram and one 30-gram batches of microspheres had highly similar size distributions. 90-95% of the microspheres were 22-36 μm in diameter, with 60-70% at 26-34 μm in diameter. 20-30% were 28-32 μm. Batches ranging from 1 gram to 30 grams also showed a similar size distribution (FIG. 35).

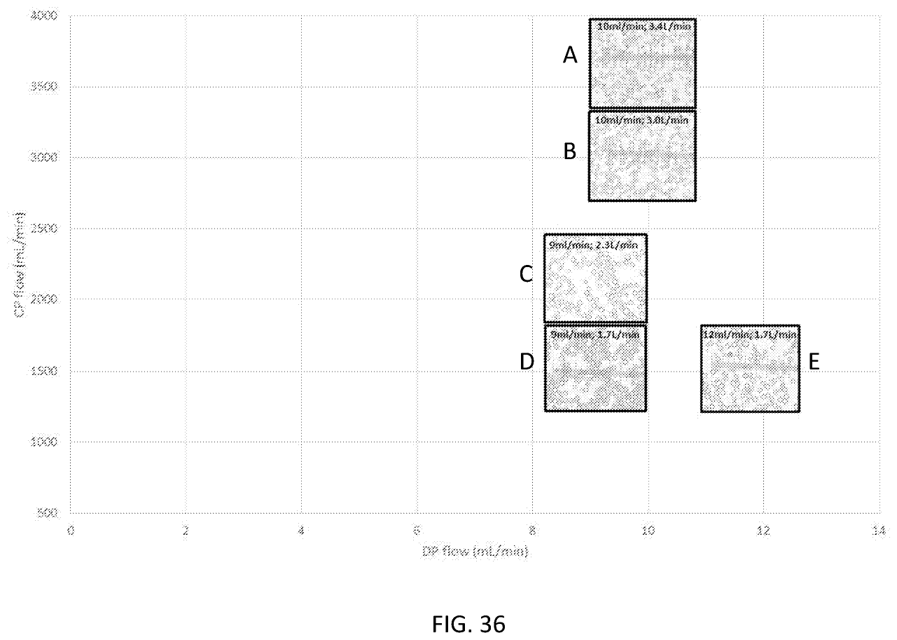

The effects of DP and CP flow rates on microsphere size/shape are shown in FIG. 36. A flow rate of 10 mL/min for the DP and 3.4 L/min for the CP generated the target size of microspheres, as shown also in FIGS. 34 & 35.

Although the foregoing disclosure has been described in some detail by way of illustration and example for purposes of clarity of understanding, the descriptions and examples should not be construed as limiting the scope of the present disclosure. The disclosures of all patent and scientific literature cited herein are expressly incorporated in their entirety by reference.

What is claimed is:

1. A microsphere, comprising:
a therapeutic compound or pharmaceutically acceptable salt thereof having a first pI; and
a first polymer, wherein the polymer has a second pI at least 1.5 units lower than the first pI;
wherein the microsphere is a single emulsion microsphere.

2. The microsphere of claim 1, further comprising a second polymer, wherein first polymer has a lower molecular weight than the second polymer.

3. The microsphere of claim 2, wherein the pharmacokinetic burst in serum per mg of the therapeutic compound or salt for the microsphere is equal to or less than the pharmacokinetic burst in serum per mg of the therapeutic compound or salt for a reference microsphere.

4. The microsphere of claim 3, wherein the reference microsphere comprises the second polymer but lacks the first polymer.

5. The microsphere of claim 3 or claim 4, wherein the reference microsphere comprises the therapeutic compound or salt at a lower loading level than the microsphere.

6. The microsphere of any one of claims 3-5, wherein the reference microsphere is a double emulsion microsphere.

7. The microsphere of any one of claims 1-6, further comprising a polyol.

8. The microsphere of claim 7, wherein the pharmacokinetic burst in serum per mg of the therapeutic compound or salt for the microsphere is equal to or less than the pharmacokinetic burst in serum per mg of the therapeutic compound or salt for a reference microsphere, wherein the reference microsphere comprises the therapeutic compound and the polymer but lacks the polyol.

9. The microsphere of claim 7 or claim 8, wherein degradation of the therapeutic compound or salt in the microsphere is less than degradation of the therapeutic compound or salt in a reference microsphere, wherein the reference microsphere comprises the therapeutic compound and the polymer but lacks the polyol.

10. The microsphere of any one of claims 3-9, wherein burst AUC in serum per mg of the therapeutic compound or salt for the microsphere is equal to or less than burst AUC in serum per mg of the therapeutic compound or salt for the reference microsphere.

11. The microsphere of any one of claims 3-9, wherein burst Cmax in serum per mg of the therapeutic compound or salt for the microsphere is less than burst Cmax in serum per mg of the therapeutic compound or salt for the reference microsphere.

12. The microsphere of any one of claims 2-11, wherein the first polymer has a molecular weight at least 10 kD lower than the second polymer.

13. The microsphere of any one of claims 1-12, wherein the therapeutic compound or salt is greater than 5% by total weight of the microsphere.

14. The microsphere of any one of claims 1-13, wherein the first polymer comprises at least one anionic terminus.

15. The microsphere of any one of claims 1-14, wherein the first polymer comprises poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer.

16. The microsphere of any one of claims 1-15, wherein the therapeutic compound or salt comprises at least one cationic moiety.

17. The microsphere of any one of claims 1-16, wherein the microsphere is produced from a feed comprising the first polymer at a concentration of at least about 150 mg/mL and the therapeutic compound or salt at a concentration of at least about 10 mg/mL.

18. The microsphere of claim 17, wherein the microsphere is produced from a feed comprising the first polymer at a concentration of at least about 200 mg/mL and the therapeutic compound or salt at a concentration of at least about 20 mg/mL.

19. The microsphere of any one of claims 2-18, wherein the microsphere is produced from a feed comprising the first polymer and the second polymer at a total concentration of at least about 150 mg/mL and the therapeutic compound or salt at a concentration of at least about 10 mg/mL.

20. The microsphere of claim 19, wherein the microsphere is produced from a feed comprising the first polymer and the second polymer at a total concentration of at least about 200 mg/mL and the therapeutic compound or salt at a concentration of at least about 20 mg/mL.

21. The microsphere of any one of claims 1-20, wherein the molecular weight of the first polymer is less than or equal to 17 kD.

22. The microsphere of any one of claims 2-21, wherein the second polymer comprises poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer.

23. The microsphere of any one of claims 2-22, wherein the first and the second polymers both comprise PLGA.

24. The microsphere of any one of claims 2-23, wherein the microsphere comprises the first polymer and the second polymer at a ratio of between about 20:80 and about 80:20 (first polymer:second polymer).

25. The microsphere of claim 24, wherein the microsphere comprises the first polymer and the second polymer at a ratio of about 75:25 (first polymer:second polymer).

26. The microsphere of claim 24, wherein the microsphere comprises the first polymer and the second polymer at a ratio of about 65:35 (first polymer:second polymer).

27. The microsphere of any one of claims 7-26, wherein the polyol is glycerol.

28. The microsphere of any one of claims 1-27, wherein the therapeutic compound comprises a therapeutic peptide.

29. The microsphere of claim 28, wherein the therapeutic peptide comprises at least two amino-containing amino acid side chains.

30. The microsphere of claim 28 or claim 29, wherein the therapeutic peptide has a length from 6 to 40 amino acids.

31. The microsphere of any one of claims 28-30, wherein the therapeutic peptide has a length of 8 amino acids.

32. The microsphere of any one of claims 28-31, wherein the therapeutic peptide is cyclic.

33. The microsphere of any one of claims 28-32, wherein the therapeutic peptide is a somatostatin analog or a pharmaceutically acceptable salt thereof.

34. The microsphere of any one of claims 28-33, wherein the therapeutic peptide is selected from the group consisting of somatostatin (SST-28), SST-14, lanreotide, octreotide, vapreotide, pasireotide, and pharmaceutically acceptable salts of any of the foregoing.

35. The microsphere of any one of claims 1-27, wherein the therapeutic compound comprises a glucocorticoid, JAK inhibitor, or mTOR inhibitor.

36. The microsphere of claim 35, wherein the therapeutic compound comprises a JAK inhibitor that inhibits JAK1, JAK3, JAK1 and JAK3, or JAK1, JAK2, and JAK3.

37. The microsphere of claim 35, wherein the therapeutic compound comprises a JAK inhibitor selected from the group consisting of ruxolitinib, tofacitinib, oclacitinib, baricitinib, filgotinib, gandotinib, lestaurtinib, momelotinib, pacritinib, PF-04965842, upadacitinib, peficitinib, fedratinib, cucurbitacin I, decernotinib, INCB018424, AC430, BMS-0911543, GSK2586184, VX-509, R348, AZD1480, CHZ868, PF-956980, AG490, WP-1034, JAK3 inhibitor IV, atiprimod, FM-381, SAR20347, AZD4205, ARN4079, NIBR-3049, PRN371, PF-06651600, JAK3i, JAK3 inhibitor 31, PF-06700841, NC1153, EP009, Gingerenone A, JANEX-1, cercosporamide, JAK3-IN-2, PF-956980, Tyk2-IN-30, Tyk2-IN-2, JAK3-IN1, WHI-P97, TG-101209, AZ960, NVP-BSK805, NSC 42834, FLLL32, SD 1029, WIH-P154, WHI-P154, TCS21311, JAK3-IN-1, JAK3-IN-6, JAK3-IN-7, XL019, MS-1020, AZD1418, WP1066, CEP33779, ZM 449829, SHR0302, JAK1-IN-31, WYE-151650, EXEL-8232, solcitinib, itacitinib, cerdulatinib, PF-06263276, delgotinib, AS2553627, JAK-IN-35, ASN-002, AT9283, diosgenin, JAK inhibitor 1, JAK-IN-1, LFM-A13, NS-018, RGB-286638, SB1317, curcumol, Go6976, JAK2 inhibitor G5-7, myricetin, and pyridine 6.

38. The microsphere of any one of claims 1-37, wherein the microsphere is substantially free of small hydrocarbons and/or silicon oil.

39. A method of preparing a single emulsion microsphere, comprising the steps of:
a) combining a first solvent and a therapeutic compound or pharmaceutically acceptable salt thereof to form a first mixture, wherein the compound or salt has a first pI;
b) combining a second solvent and a first polymer to form a second mixture, wherein the polymer has a second pI at least 1.5 units lower than the first pI;
c) combining the first and second mixtures to form a feed;
d) dispersing the combined first and second mixtures of step (c) into an aqueous continuous phase to form a droplet; and
e) hardening the droplet formed in step (d) to form the single emulsion microsphere.

40. The method of claim 39, wherein step b) further comprises combining a second polymer with the second solvent and the first polymer to form the mixture.

41. The method of claim 40, wherein the first polymer has a lower molecular weight than the second polymer.

42. The method of claim 41, wherein the pharmacokinetic burst in serum per mg of the therapeutic compound or salt for the microsphere is equal to or less than pharmacokinetic burst in serum per mg of the therapeutic compound or salt for a reference microsphere.

43. The method of claim 42, wherein the reference microsphere comprises the second polymer but lacks the first polymer.

44. The method of claim 42 or claim 43, wherein the reference microsphere comprises the therapeutic compound or salt at a lower loading level than the microsphere formed in step (e).

45. The method of claim 44, wherein the microsphere formed in step (e) does not induce a burst penalty as compared with the reference microsphere.

46. The method of claim 44 or claim 45, wherein the reference microsphere is a double emulsion microsphere.

47. The method of any one of claims 39-46, wherein the feed further comprises a polyol.

48. The method of claim 47, wherein the polyol is solubilized in the feed.

49. The method of claim 47 or claim 48, wherein the pharmacokinetic burst in serum per mg of the therapeutic compound or salt for the microsphere is less than the pharmacokinetic burst in serum per mg of the therapeutic compound or salt for a reference microsphere, wherein the reference microsphere comprises the therapeutic compound and the polymer but lacks the polyol.

50. The method of any one of claims 47-49, wherein degradation of the therapeutic compound or salt in the microsphere is less than degradation of the therapeutic compound or salt in a reference microsphere, wherein the reference microsphere comprises the therapeutic compound and the polymer but lacks the polyol.

51. The method of any one of claims 42-50, wherein burst AUC in serum per mg of the therapeutic compound or salt for the microsphere is equal to or less than burst AUC in serum per mg of the therapeutic compound or salt for the reference microsphere.

52. The method of any one of claims 42-50, wherein burst Cmax in serum per mg of the therapeutic compound or salt for the microsphere is less than burst Cmax in serum per mg of the therapeutic compound or salt for the reference microsphere.

53. The method of any one of claims 40-52, wherein the first polymer has a molecular weight at least 10 kD lower than the second polymer.

54. The method of any one of claims 39-53, wherein the microsphere comprises greater than 5% by total weight of the therapeutic compound or salt.

55. The method of any one of claims 39-54, wherein the first polymer comprises at least one anionic terminus.

56. The method of any one of claims 39-55, wherein the first polymer comprises poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer.

57. The method of any one of claims 39-56, wherein the therapeutic compound or salt comprises at least one cationic moiety.

58. The method of any one of claims 39-57, wherein the feed comprises the first polymer at a concentration of at least about 150 mg/mL and the therapeutic compound or salt at a concentration of at least about 10 mg/mL.

59. The method of claim 58, wherein the feed comprises the first polymer at a concentration of at least about 200 mg/mL and the therapeutic compound or salt at a concentration of at least about 20 mg/mL.

60. The method of any one of claims 39-57, wherein the feed comprises the first polymer and the second polymer at a total concentration of at least about 150 mg/mL and the therapeutic compound or salt at a concentration of at least about 10 mg/mL.

61. The method of claim 60, wherein the feed comprises the first polymer and the second polymer at a total concentration of at least about 200 mg/mL and the therapeutic compound or salt at a concentration of at least about 20 mg/mL.

62. The method of any one of claims 39-61, wherein the molecular weight of the first polymer is less than or equal to 17 kD.

63. The method of any one of claims 40-62, wherein the second polymer comprises poly(lactic-co-glycolic acid) (PLGA), polylactic acid (PLA), polyglycolide, poly(glycolide-co-lactide) (PLG), polyhydroxybutyrate, poly(sebacic acid), polyphosphazene, poly[(lactide-co-ethylene glycol)-co-ethyloxyphosphate], PLA-polyethyleneglycol (PEG)-PLA triblock copolymer, or PLG-PEG-PLG triblock copolymer.

64. The method of any one of claims 40-63, wherein the first and the second polymers both comprise PLGA, and wherein the molecular weight of the first polymer is at least 10 kD lower than the molecular weight of the second polymer.

65. The method of any one of claims 40-64, wherein the microsphere comprises the first polymer and the second polymer at a ratio of between about 20:80 and about 80:20 (first polymer:second polymer).

66. The method of claim 65, wherein the microsphere comprises the first polymer and the second polymer at a ratio of about 75:25 (first polymer:second polymer).

67. The method of claim 65, wherein the microsphere comprises the first polymer and the second polymer at a ratio of about 65:35 (first polymer:second polymer).

68. The method of any one of claims 47-67, wherein the feed comprises the polyol at a concentration of about between about 0.3 mg/mL and about 1.2 mg/mL.

69. The method of any one of claims 47-68, wherein the feed comprises the polyol at a concentration of about 0.9 mg/mL.

70. The method of any one of claims 47-69, wherein the polyol is glycerol.

71. The method of any one of claims 39-70, wherein the therapeutic compound comprises a therapeutic peptide.

72. The method of any one of claims 39-71, wherein the therapeutic peptide comprises at least two amino-containing amino acid side chains.

73. The method of any one of claims 39-72, wherein the therapeutic peptide has a length from 6 to 40 amino acids.

74. The method of any one of claims 39-73, wherein the therapeutic peptide has a length of 8 amino acids.

75. The method of any one of claims 39-74, wherein the therapeutic peptide is cyclic.

76. The method of any one of claims 39-75, wherein the therapeutic peptide is a somatostatin analog or a pharmaceutically acceptable salt thereof.

77. The method of any one of claims 39-76, wherein the therapeutic peptide is selected from the group consisting of somatostatin (SST-28), SST-14, lanreotide, octreotide, vapreotide, pasireotide, and pharmaceutically acceptable salts of any of the foregoing.

78. The method of any one of claims 39-70, wherein the therapeutic compound comprises a glucocorticoid, JAK inhibitor, or mTOR inhibitor.

79. The method of claim 78, wherein the therapeutic compound comprises a JAK inhibitor that inhibits JAK1, JAK3, JAK1 and JAK3, or JAK1, JAK2, and JAK3.

80. The method of claim 78, wherein the therapeutic compound comprises a JAK inhibitor selected from the group consisting of ruxolitinib, tofacitinib, oclacitinib, baricitinib, filgotinib, gandotinib, lestaurtinib, momelotinib, pacritinib, PF-04965842, upadacitinib, peficitinib, fedratinib, cucurbitacin I, decernotinib, INCB018424, AC430, BMS-0911543, GSK2586184, VX-509, R348, AZD1480, CHZ868, PF-956980, AG490, WP-1034, JAK3 inhibitor IV, atiprimod, FM-381, SAR20347, AZD4205, ARN4079, NIBR-3049, PRN371, PF-06651600, JAK3i, JAK3 inhibitor 31, PF-06700841, NC1153, EP009, Gingerenone A, JANEX-1, cercosporamide, JAK3-IN-2, PF-956980, Tyk2-IN-30, Tyk2-IN-2, JAK3-IN1, WHI-P97, TG-101209, AZ960, NVP-BSK805, NSC 42834, FLLL32, SD 1029, WIH-P154, WHI-P154, TCS21311, JAK3-IN-1, JAK3-IN-6, JAK3-IN-7, XL019, MS-1020, AZD1418, WP1066, CEP33779, ZM 449829, SHR0302, JAK1-IN-31, WYE-151650, EXEL-8232, solcitinib, itacitinib, cerdulatinib, PF-06263276, delgotinib, AS2553627, JAK-IN-35, ASN-002, AT9283, diosgenin, JAK inhibitor 1, JAK-IN-1, LFM-A13, NS-018, RGB-286638, SB1317, curcumol, Go6976, JAK2 inhibitor G5-7, myricetin, and pyridine 6.

81. The method of any one of claims 39-80, further comprising adjusting the pH of the aqueous continuous phase into which the feed is dispersed in step (d).

82. The method of claim 81, wherein the pH of the aqueous continuous phase is adjusted with a buffer solution selected from the group consisting of glycine, glycylglycine, tricine, HEPES, MOPS, sulfonate, ammonia, potassium phosphate, CHES, borate, TAPS, Tris, bicine, TAPSO, TES, and Tris buffer solutions.

83. The method of claim 81, wherein the pH of the aqueous continuous phase is adjusted with a buffer solution selected from the group consisting of glycylglycine, bicine, and tricine.

84. The method of any one of claims 81-83, wherein the pH of the aqueous continuous phase is adjusted to the first pI minus 0.5 or greater.

85. The method of claim 84, wherein the pH of the aqueous continuous phase is adjusted to about 8 to about 9.5.

86. The method of claim 85, wherein the pH of the aqueous continuous phase is adjusted to about 9.

87. The method of claim 84, wherein the pH of the aqueous continuous phase is adjusted to about 7.5 to about 8.5.

88. The method of claim 87, wherein the pH of the aqueous continuous phase is adjusted to about 8.

89. The method of any one of claims 39-88, wherein the droplet is allowed to harden in (e) for at least about 120 minutes.

90. The method of any one of claims 39-89, wherein a plurality of microspheres are produced, and wherein at least 90% of the microspheres of the plurality are 22-36 μm in diameter.

91. The method of any one of claims 39-89, wherein a plurality of microspheres are produced, and wherein at least 60% of the microspheres of the plurality are 26-34 μm in diameter.

92. The method of any one of claims 39-91, further comprising, after step (d) and prior to step (e), washing the microsphere in a second aqueous continuous phase.

93. The method of claim 92, further comprising, after washing the microsphere in a second aqueous continuous phase, performing size-selective filtration on the microsphere.

94. The method of claim 92 or claim 93, wherein the second aqueous continuous phase has the same composition as the first aqueous continuous phase.

95. The method of any one of claims 39-94, further comprising, after step (e), washing the microsphere in an aqueous alcohol solution.

96. The method of claim 95, wherein the aqueous alcohol solution comprises an aliphatic alcohol at a concentration of between about 1% and about 20%.

97. The method of claim 96, wherein the aqueous alcohol solution comprises ethanol at a concentration of about 10%.

98. The method of any one of claims 95-97, wherein the aqueous alcohol solution further comprises a buffer.

99. The method of claim 98, wherein the buffer is an acetate buffer.

100. The method of claim 98 or claim 99, wherein the aqueous alcohol solution is buffered to a pH of about 4.

101. The method of any one of claims 39-100, further comprising, after step (e), lyophilizing the microsphere.

102. The method of any one of claims 39-100, further comprising, after step (e), spray drying the microsphere.

103. The method of any one of claims 39-102, wherein the feed of step (c) comprises a ratio of between 10:1 and 10:3 (first polymer:therapeutic compound or salt) by weight.

104. The method of any one of claims 39-103, wherein the feed of step (c) comprises the therapeutic compound or salt at a concentration of between about 10 mg/mL and about 60 mg/mL by weight.

105. The method of any one of claims 39-104, wherein hardening the droplet in step (e) comprises exacervation.

106. The method of any one of claims 39-105, wherein the first solvent comprises ethanol, propanol, or methanol.

107. The method of any one of claims 39-106, wherein the second solvent comprises dichloromethane, chloroform, or ethyl acetate.

108. The method of any one of claims 39-107, wherein the method does not comprise the addition of a small hydrocarbon and/or silicon oil.

109. The method of any one of claims 39-108, wherein dispersing the feed in step (d) comprises use of a membrane.

110. The method of claim 109, wherein the membrane comprises a material treated to increase hydrophilicity of the membrane.

111. The method of claim 110, wherein the membrane is coated with a hydrophilic polymer.

112. The method of any one of claims 109-111, wherein the membrane comprises stainless steel, tantalum, tungsten, molybdenum, manganese, tin, zinc, or an alloy thereof.

113. The method of any one of claims 109-111, wherein the membrane comprises porous glass or a ceramic.

114. The method of any one of claims 109-113, wherein the membrane comprises pores having a size from about 5 μm to about 50 μm.

115. The method of claim 114, wherein the membrane comprises pores having a size from about 5 μm to about 20 μm.

116. The method of any one of claims 109-115, wherein the membrane comprises pores having a size from about 5 μm to about 50 μm, and wherein the feed is dispersed in step (d) at a flow rate of about 130 nL/min/pore.

117. The method of any one of claims 109-115, wherein the feed is dispersed in step (d) at a flow rate of between about 0.1 nLmin⁻¹ μm⁻² (pore size) and about 1 nLmin⁻¹ μm⁻² (pore size).

118. The method of any one of claims 39-117, wherein the feed is dispersed in step (d) by applying shear force.

119. The method of claim 118, wherein the shear force is between about 500 s⁻¹ and about 40,000 s⁻¹.

120. The method of any one of claims 39-119, wherein the aqueous continuous phase further comprises a surfactant.

121. The method of claim 120, wherein the surfactant is selected from the group consisting of polysorbate 20, polysorbate 80, poloxamer, and polyvinyl alcohol (PVA).

122. The method of claim 120 or claim 121, wherein the concentration of the surfactant in the aqueous continuous phase is from 0.05% to 1% (w/w).

123. The method of claim 122, wherein the concentration of the surfactant in the aqueous continuous phase is about 0.5% (w/w).

124. A microsphere produced by the method of any one of claims 39-123.

125. A pharmaceutical composition comprising the microsphere of any one of claims 1-38 and 124.

126. A method of treating a condition, comprising:
administering to the individual a therapeutically effective amount of the microsphere of any one of claims 1-38 and 124 or the composition of claim 125,
wherein the condition is selected from the group consisting of acromegaly, carcinoid tumors, vasoactive intestinal peptide secreting tumors, diarrhea associated with acquired immune deficiency syndrome (AIDS), diarrhea associated with chemotherapy, diarrhea associated with radiation therapy, dumping syndrome, adrenal gland neuroendocrine tumors, bowel obstruction, enterocutaneous fistulae, gastrinoma, acute bleeding of gastroesophageal varices, islet cell tumors, lung neuroendocrine tumors, malignancy, meningiomas, gastrointestinal tract neuroendocrine tumors, thymus neuroendocrine tumors, pancreatic fistulas, pancreas neuroendocrine tumors, pituitary adenomas, short-bowel syndrome, small or large cell neuroendocrine tumors, thymomas and thymic carcinomas, Zollinger Ellison syndrome, acute pancreatitis, breast cancer, chylothorax, congenital lymphedema, diabetes mellitus, gastric paresis, hepatocellular carcinoma, non-variceal upper gastrointestinal bleeding, obestity, pancreaticoduodenectomy, prostate cancer, protein-losing enteropathy, small cell lung cancer, thyroid cancer, thyroid eye disease, vascular (arterio-venous) malformations of the gastrointestinal tract, polycystic kidney disease, Cushing's disease, GHRH-producing tumors, and other conditions resulting in abnormally elevated growth hormone, insulin, or glucagon levels in an individual in need thereof.

127. A method of treating growth hormone deficiency, comprising:
administering to an individual in need thereof a therapeutically effective amount of the microsphere of any one of claims 1-38 and 124 or the composition of claim 125.

128. The method of claim 126 or claim 127, wherein the microsphere or composition is administered to the individual by injection.

129. The method of claim 128, wherein the injection is a subcutaneous or intramuscular injection.

130. A method of treating alopecia, comprising:
administering to an individual in need thereof a therapeutically effective amount of the microsphere of any one of claims 1-38 and 124 or the composition of claim 125, wherein the therapeutic compound or pharmaceutically acceptable salt thereof is a JAK inhibitor.

131. The method of claim 130, wherein the therapeutic compound comprises a JAK inhibitor that inhibits JAK1, JAK3, JAK1 and JAK3, or JAK1, JAK2, and JAK3.

132. The method of claim 130, wherein the therapeutic compound comprises a JAK inhibitor selected from the group consisting of ruxolitinib, tofacitinib, oclacitinib, baricitinib, filgotinib, gandotinib, lestaurtinib, momelotinib, pacritinib, PF-04965842, upadacitinib, peficitinib, fedratinib, cucurbitacin I, decernotinib, INCB018424, AC430, BMS-0911543, GSK2586184, VX-509, R348, AZD1480, CHZ868, PF-956980, AG490, WP-1034, JAK3 inhibitor IV, atiprimod, FM-381, SAR20347, AZD4205, ARN4079, NIBR-3049, PRN371, PF-06651600, JAK3i, JAK3 inhibitor 31, PF-06700841, NC1153, EP009, Gingerenone A, JANEX-1, cercosporamide, JAK3-IN-2, PF-956980, Tyk2-IN-30, Tyk2-IN-2, JAK3-IN1, WHI-P97, TG-101209, AZ960, NVP-BSK805, NSC 42834, FLLL32, SD 1029, WIH-P154, WHI-P154, TCS21311, JAK3-IN-1, JAK3-IN-6, JAK3-IN-7, XL019, MS-1020, AZD1418, WP1066, CEP33779, ZM 449829, SHR0302, JAK1-IN-31, WYE-151650, EXEL-8232, solcitinib, itacitinib, cerdulatinib, PF-06263276, delgotinib, AS2553627, JAK-IN-35, ASN-002, AT9283, diosgenin, JAK inhibitor 1, JAK-IN-1, LFM-A13, NS-018, RGB-286638, SB1317, curcumol, Go6976, JAK2 inhibitor G5-7, myricetin, and pyridine 6.

133. The method of any one of claims 130-132, wherein the microsphere or composition is administered to the individual by dermal or subdermal injection.

134. The method of any one of claims 126-133, wherein the individual is a human.
